SEQUENCE LISTING (222 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 93 aa, Homo sapiens
SDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 3 - protein, 92 aa, Homo sapiens
DVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 4 - protein, 91 aa, Homo sapiens
VPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 5 - protein, 90 aa, Homo sapiens
PRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 6 - protein, 89 aa, Homo sapiens
RDLEVVAATPTSLLISWDAPAVTVRYYRITYGETGGNSPVQEFTVPGSKSTATISGLKPGVDYTITVYAVTGRGDSPASSKPISINYRT

SEQ ID NO: 7 - protein, 88 aa, Homo sapiens
DLEVVAATPTSLLISWDAPAVTVRYYRITYGETGGNSPVQEFTVPGSKSTATISGLKPGVDYTITVYAVTGRGDSPASSKPISINYRT

SEQ ID NO: 8 - protein, 87 aa, Homo sapiens
LEVVAATPTSLLISWDAPAVTVRYYRITYGETGGNSPVQEFTVPGSKSTATISGLKPGVDYTITVYAVTGRGDSPASSKPISINYRT

SEQ ID NO: 9 - protein, 101 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 10 - protein, 101 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 11 - protein, 100 aa, Homo sapiens
SDVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 12 - protein, 100 aa, Homo sapiens
SDVPRDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 13 - protein, 99 aa, Homo sapiens
DVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 14 - protein, 99 aa, Homo sapiens
DVPRDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 15 - protein, 98 aa, Homo sapiens
VPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 16 - protein, 98 aa, Homo sapiens
VPRDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 17 - protein, 97 aa, Homo sapiens
PRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 18 - protein, 97 aa, Homo sapiens
PRDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 19 - protein, 96 aa, Homo sapiens
RDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 20 - protein, 96 aa, Homo sapiens
RDLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 21 - protein, 95 aa, Homo sapiens
DLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 22 - protein, 95 aa, Homo sapiens
DLEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 23 - protein, 94 aa, Homo sapiens
LEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 24 - protein, 94 aa, Homo sapiens
LEVVAATPTSLLISWDAPAVTVRYYRITYGETGGNSPVQEFTVPGSKSTATISGLKPGVDYTITVYAVTGRGDSPASSKPISINYRTEIEKPSQ

SEQ ID NO: 25 - protein, 166 aa, Artificial Sequence
  Description of Artificial Sequence Synthetic polypeptide
VSDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 26 - protein, 165 aa, Artificial Sequence
  Description of Artificial Sequence Synthetic polypeptide
SDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 27 - protein, 164 aa, Artificial Sequence
  Description of Artificial Sequence Synthetic polypeptide
DVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 28 - protein, 163 aa, Artificial Sequence
  Description of Artificial Sequence Synthetic polypeptide
VPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 29 - protein, 162 aa, Artificial Sequence
  Description of Artificial Sequence Synthetic polypeptide
PRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 30 - protein, 161 aa, Artificial Sequence
  Description of Artificial Sequence Synthetic polypeptide
RDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 31 - protein, 160 aa, Artificial Sequence
  Description of Artificial Sequence Synthetic polypeptide
DLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 32 - protein, 159 aa, Artificial Sequence
  Description of Artificial Sequence Synthetic polypeptide
LEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 33 - protein, 173 aa, Artificial Sequence
  Description of Artificial Sequence Synthetic polypeptide
VSDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 34 - protein, 173 aa, Artificial Sequence
  Description of Artificial Sequence Synthetic polypeptide
VSDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 35 - protein, 172 aa, Artificial Sequence
  Description of Artificial Sequence Synthetic polypeptide
SDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 36 - protein, 172 aa, Artificial Sequence
  Description of Artificial Sequence Synthetic polypeptide
SDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 37 - protein, 171 aa, Artificial Sequence
  Description of Artificial Sequence Synthetic polypeptide
DVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 38 - protein, 171 aa, Artificial Sequence
  Description of Artificial Sequence Synthetic polypeptide
DVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 39 - protein, 170 aa, Artificial Sequence
  Description of Artificial Sequence Synthetic polypeptide
VPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 40 - protein, 170 aa, Artificial Sequence
  Description of Artificial Sequence Synthetic polypeptide
VPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 41 - protein, 169 aa, Artificial Sequence
  Description of Artificial Sequence Synthetic polypeptide
PRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 42 - protein, 169 aa, Artificial Sequence
  Description of Artificial Sequence Synthetic polypeptide
PRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 43 - protein, 168 aa, Artificial Sequence
  Description of Artificial Sequence Synthetic polypeptide
RDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 44 - protein, 168 aa, Artificial Sequence
  Description of Artificial Sequence Synthetic polypeptide
RDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 45 - protein, 167 aa, Artificial Sequence
  Description of Artificial Sequence Synthetic polypeptide
DLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 46 - protein, 167 aa, Artificial Sequence
  Description of Artificial Sequence Synthetic polypeptide
DLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 47 - protein, 166 aa, Artificial Sequence
  Description of Artificial Sequence Synthetic polypeptide
LEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIDKPSQ

SEQ ID NO: 48 - protein, 166 aa, Artificial Sequence
  Description of Artificial Sequence Synthetic polypeptide
LEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRTEIEKPSQ

SEQ ID NO: 49 - protein, 87 aa, Homo sapiens; identical to SEQ ID NO: 8

SEQ ID NO: 50 - protein, 159 aa, Artificial Sequence; identical to SEQ ID NO: 32
  Description of Artificial Sequence Synthetic polypeptide

SEQ ID NO: 51 - protein, 7 aa, Homo sapiens
VSDVPRD

SEQ ID NO: 52 - protein, 6 aa, Homo sapiens
SDVPRD

SEQ ID NO: 53 - protein, 5 aa, Homo sapiens
DVPRD

SEQ ID NO: 54 - protein, 4 aa, Homo sapiens
VPRD

SEQ ID NO: 55 - protein, 7 aa, Homo sapiens
EIDKPSQ

SEQ ID NO: 56 - protein, 7 aa, Homo sapiens
EIEKPSQ

SEQ ID NO: 57 - protein, 10 aa, Homo sapiens
MGVSDVPRDL

SEQ ID NO: 58 - protein, 9 aa, Homo sapiens
GVSDVPRDL

SEQ ID NO: 59 - protein, 9 aa, Artificial Sequence
  Description of Artificial Sequence Synthetic peptide
MGSDVPRDL

SEQ ID NO: 60 - protein, 8 aa, Artificial Sequence
  Description of Artificial Sequence Synthetic peptide
MGDVPRDL

SEQ ID NO: 61 - protein, 7 aa, Artificial Sequence
  Description of Artificial Sequence Synthetic peptide
MGVPRDL

SEQ ID NO: 62 - protein, 6 aa, Artificial Sequence
  Description of Artificial Sequence Synthetic peptide
MGPRDL

SEQ ID NO: 63 - protein, 5 aa, Artificial Sequence
  Description of Artificial Sequence Synthetic peptide
MGRDL

SEQ ID NO: 64 - protein, 4 aa, Artificial Sequence
  Description of Artificial Sequence Synthetic peptide
MGDL

SEQ ID NO: 65 - protein, 6 aa, Homo sapiens
MASTSG

SEQ ID NO: 66 - protein, 4 aa, Homo sapiens
EIEK

SEQ ID NO: 67 - protein, 5 aa, Homo sapiens
EGSGC

SEQ ID NO: 68 - protein, 7 aa, Homo sapiens
EIEKPCQ

SEQ ID NO: 69 - protein, 5 aa, Homo sapiens
EIEKP

SEQ ID NO: 70 - protein, 6 aa, Homo sapiens
EIEKPS

SEQ ID NO: 71 - protein, 6 aa, Homo sapiens
EIEKPC

SEQ ID NO: 72 - protein, 6 aa, Artificial Sequence
  Description of Artificial Sequence Synthetic 6xHis tag
HHHHHH

SEQ ID NO: 73 - protein, 4 aa, Homo sapiens
EIDK

SEQ ID NO: 74 - protein, 7 aa, Homo sapiens
EIDKPCQ

SEQ ID NO: 75 - protein, 91 aa, Artificial Sequence
  Description of Artificial Sequence Synthetic polypeptide
MPAPTDLRFTNETPSSLLISWTPPRVQITGYIIRYGPVGSDGRVKEFTVPPSVSSATITGLKPGTEYTISVIALKDNQESEPLRGRVTTGG

SEQ ID NO: 76 - protein, 4 aa, Artificial Sequence
  Description of Artificial Sequence Synthetic peptide
TPSS

SEQ ID NO: 77 - protein, 7 aa, Artificial Sequence
  Description of Artificial Sequence Synthetic peptide
TPPRVQI

SEQ ID NO: 78 - protein, 6 aa, Artificial Sequence
  Description of Artificial Sequence Synthetic peptide
VGSDGR

SEQ ID NO: 79 - protein, 4 aa, Artificial Sequence
  Description of Artificial Sequence Synthetic peptide
PSVS

SEQ ID NO: 80 - protein, 5 aa, Artificial Sequence
  Description of Artificial Sequence Synthetic peptide
GLKPG

SEQ ID NO: 81 - protein, 8 aa, Artificial Sequence
  Description of Artificial Sequence Synthetic peptide
KDNQESEP

SEQ ID NO: 82 - protein, 89 aa, Artificial Sequence
  Description of Artificial Sequence Synthetic polypeptide
LDAPTDLQVTNVTDTSITVSWTPPSATITGYRITYTPSNGPGEPKELTVPPSSTSVTITGITPGVEYVVSVYALKDNQESPPLVGTCTT

SEQ ID NO: 83 - protein, 89 aa, Artificial Sequence
  Description of Artificial Sequence Synthetic polypeptide
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 84 - protein, 4 aa, Artificial Sequence
  Description of Artificial Sequence Synthetic peptide
TEDS

SEQ ID NO: 85 - protein, 7 aa, Artificial Sequence
  Description of Artificial Sequence Synthetic peptide
TAPDAAF

SEQ ID NO: 86 - protein, 6 aa, Artificial Sequence
  Description of Artificial Sequence Synthetic peptide
SEKVGE

SEQ ID NO: 87 - protein, 4 aa, Artificial Sequence
  Description of Artificial Sequence Synthetic peptide
GSER

SEQ ID NO: 88 - protein, 5 aa, Artificial Sequence
  Description of Artificial Sequence Synthetic peptide
GLKPG

SEQ ID NO: 89 - protein, 7 aa, Artificial Sequence
  Description of Artificial Sequence Synthetic peptide
KGGHRSN

SEQ ID NO: 90 - protein, 7 aa, Artificial Sequence
  Description of Artificial Sequence Synthetic peptide
GPGPGPG

SEQ ID NO: 91 - protein, 11 aa, Artificial Sequence
  Description of Artificial Sequence Synthetic peptide
GPGPGPGPGPG

SEQ ID NO: 92 - protein, 6 aa, Artificial Sequence
  Description of Artificial Sequence Synthetic peptide
PAPAPA

SEQ ID NO: 93 - protein, 12 aa, Artificial Sequence
  Description of Artificial Sequence Synthetic peptide
PAPAPAPAPAPA

SEQ ID NO: 94 - protein, 18 aa, Artificial Sequence
  Description of Artificial Sequence Synthetic peptide
PAPAPAPAPAPAPAPAPA

SEQ ID NO: 95 - protein, 10 aa, Artificial Sequence
  Description of Artificial Sequence Synthetic peptide
GSGSGSGSGS

SEQ ID NO: 96 - protein, 12 aa, Artificial Sequence
  Description of Artificial Sequence Synthetic peptide
GSGSGSGSGSGS

SEQ ID NO: 97 - protein, 20 aa, Artificial Sequence
  Description of Artificial Sequence Synthetic peptide
GSGSGSGSGSGSGSGSGSGS

SEQ ID NO: 98 - protein, 15 aa, Artificial Sequence
  Description of Artificial Sequence Synthetic peptide
GGGGSGGGGSGGGGS

SEQ ID NO: 99 - protein, 25 aa, Artificial Sequence
  Description of Artificial Sequence Synthetic peptide
GGGGSGGGGSGGGGSGGGGSGGGGS

SEQ ID NO: 100 - protein, 15 aa, Artificial Sequence
  Description of Artificial Sequence Synthetic peptide
GGGGSGGGGSGGGSG

SEQ ID NO: 101 - protein, 609 aa, Homo sapiens [sequence omitted]

SEQ ID NO: 102 - protein, 585 aa, Homo sapiens [sequence omitted]

SEQ ID NO: 103 - protein, 46 aa, Streptococcus sp.
LAEAKVLANRELDKYGVSDYYKNLINNAKTVEGVKALIDEILAALP

SEQ ID NO: 104 - protein, 47 aa, Streptococcus sp.
SLAEAKVLANRELDKYGVSDYYKNLINNAKTVEGVKALIDEILAALP

SEQ ID NO: 105 - protein, 48 aa, Streptococcus sp.
NSLAEAKVLANRELDKYGVSDYYKNLINNAKTVEGVKALIDEILAALP

SEQ ID NO: 106 - protein, 49 aa, Streptococcus sp.
ANSLAEAKVLANRELDKYGVSDYYKNLINNAKTVEGVKALIDEILAALP

SEQ ID NO: 107 - protein, 50 aa, Streptococcus sp.
DANSLAEAKVLANRELDKYGVSDYYKNLINNAKTVEGVKALIDEILAALP

SEQ ID NO: 108 - protein, 52 aa, Streptococcus sp.
AVDANSLAEAKVLANRELDKYGVSDYYKNLINNAKTVEGVKALIDEILAALP

SEQ ID NO: 109 - protein, 53 aa, Streptococcus sp.
EAVDANSLAEAKVLANRELDKYGVSDYYKNLINNAKTVEGVKALIDEILAALP

SEQ ID NO: 110 - protein, 54 aa, Streptococcus sp.
DEAVDANSLAEAKVLANRELDKYGVSDYYKNLINNAKTVEGVKALIDEILAALP

SEQ ID NO: 111 - protein, 55 aa, Streptococcus sp.
HDEAVDANSLAEAKVLANRELDKYGVSDYYKNLINNAKTVEGVKALIDEILAALP

SEQ ID NO: 112 - protein, 56 aa, Streptococcus sp.
QHDEAVDANSLAEAKVLANRELDKYGVSDYYKNLINNAKTVEGVKALIDEILAALP

SEQ ID NO: 113 - protein, 26 aa, Streptococcus sp.
GVSDFYKNVINRAKTVEGVHALIDHI

SEQ ID NO: 114 - protein, 26 aa, Streptococcus sp.
GVSDYYKNIINRAKTVEGVRALKLHI

SEQ ID NO: 115 - protein, 26 aa, Streptococcus sp.
GVSDFYKNLINRAKTVEGVSSLKGHI

SEQ ID NO: 116 - protein, 26 aa, Streptococcus sp.
GVSDFYKSLINRAKTVEGVDALTSHI

SEQ ID NO: 117 - protein, 26 aa, Streptococcus sp.
GVSDFYKSYINRAKTVEGVHTLIGHI

SEQ ID NO: 118 - protein, 26 aa, Streptococcus sp.
GVSDFYKNLINRAKTVEGVNSLTSHI

SEQ ID NO: 119 - protein, 26 aa, Streptococcus sp.
GVSDFYKNVINKAKTVEGVEALIADI

SEQ ID NO: 120 - protein, 26 aa, Streptococcus sp.
GVSDFYKRLINKAKTVEGVNALTHHI

SEQ ID NO: 121 - protein, 26 aa, Streptococcus sp.
GVSDYYKNLINKAKTVEGVEALTLHI

SEQ ID NO: 122 - protein, 26 aa, Streptococcus sp.
GVSDFYKNLINRAKTVEGVDALIAHI

SEQ ID NO: 123 - protein, 26 aa, Streptococcus sp.
GVSDYYKNLINKAKTVEGVEALKLHI

SEQ ID NO: 124 - protein, 26 aa, Streptococcus sp.
GVSDYYKNLINRARTVEGVEALKLHI

SEQ ID NO: 125 - protein, 26 aa, Streptococcus sp.
GVSDYYKNLINKAKTVEGVQALIAHI

SEQ ID NO: 126 - protein, 26 aa, Streptococcus sp.
GVSDFYKRLINKAKTVEGVEALKLHI

SEQ ID NO: 127 - protein, 26 aa, Streptococcus sp.
GVSDYYKNIINRAKTVEGVEALKLHI

SEQ ID NO: 128 - protein, 26 aa, Streptococcus sp.
GVSDFYKNLINRAKTVEGVEALKLHI

SEQ ID NO: 129 - protein, 26 aa, Streptococcus sp.
GVSDFYKNVINKAKTVEGVEALKLHI

SEQ ID NO: 130 - protein, 26 aa, Streptococcus sp.
GVSDYYKNLINRAKTVEGVEALKLHI

SEQ ID NO: 131 - protein, 26 aa, Streptococcus sp.
GVSDYYKNLINRARTVEGVHALIDHI

SEQ ID NO: 132 - protein, 26 aa, Streptococcus sp.
GVSDYYKRLISKAKTVEGVKALISEI

SEQ ID NO: 133 - protein, 26 aa, Streptococcus sp.
GVSDFYKRLINKAKTVEGVEALKLHI

SEQ ID NO: 134 - protein, 26 aa, Streptococcus sp.
GVSDYYKRLISKAKTVEGVKALISEI

SEQ ID NO: 135 - protein, 46 aa, Streptococcus sp.
LAEAKVLANRELDKYGVSDYYKNIINRAKTVEGVRALKLHILAALP

SEQ ID NO: 136 - protein, 46 aa, Streptococcus sp.
LAEAKVLANRELDKYGVSDFYKSLINRAKTVEGVHSLTDEILAALP

SEQ ID NO: 137 - protein, 46 aa, Streptococcus sp.
LAEAKVLANRELDKYGVSDFYKSLINRAKTVEGVDALTSHILAALP

SEQ ID NO: 138 - protein, 46 aa, Streptococcus sp.
LAEAKVLANRELDKYGVSDYYKNLINRARTVEGVDTLIHDILAALP

SEQ ID NO: 139 - protein, 46 aa, Streptococcus sp.
LAEAKVLANRELDKYGVSDYYKNLINKAKTVEGVDALIAHILAALP

SEQ ID NO: 140 - protein, 46 aa, Streptococcus sp.
LAEAKVLANRELDKYGVSDFYKNLINRAKTVEGVNALISDILAALP

SEQ ID NO: 141 - protein, 46 aa, Streptococcus sp.
LAEAKVLANRELDKYGVSDFYKNLINRAKTVEGVSALIQEILAALP

SEQ ID NO: 142 - protein, 46 aa, Streptococcus sp.
LAEAKVLANRELDKYGVSDFYKNLINRAKTVEGVQSLIDHILAALP

SEQ ID NO: 143 - protein, 46 aa, Streptococcus sp.
LAEAKVLANRELDKYGVSDFYKNLINRAKTVEGVNALKSDILAALP

SEQ ID NO: 144 - protein, 46 aa, Streptococcus sp.
LAEAKVLANRELDKYGVSDFYKRLINKAKTVEGVHTLKHHILAALP

SEQ ID NO: 145 - protein, 46 aa, Streptococcus sp.
LAEAKVLANRELDKYGVSDYYKRLINRAKTVEGVHALKAHILAALP

SEQ ID NO: 146 - protein, 46 aa, Streptococcus sp.
LAEAKVLANRELDKYGVSDYYKRLINRARTVEGVDTLKHHILAALP

SEQ ID NO: 147 - protein, 46 aa, Streptococcus sp.
LAEAKVLANRELDKYGVSDFYKRVINRARTVEGVQALKAHILAALP

SEQ ID NO: 148 - protein, 46 aa, Streptococcus sp.
LAEAKVLANRELDKYGVSDFYKRLINRAKTVEGVESLKAHILAALP

SEQ ID NO: 149 - protein, 46 aa, Streptococcus sp.
LAEAKVLANRELDKYGVSDFYKRLINRARTVEGVDSLKVHILAALP

SEQ ID NO: 150 - protein, 46 aa, Streptococcus sp.
LAEAKVLANRELDKYGVSDFYKRVINRAKTVEGVSALKRHILAALP

SEQ ID NO: 151 - protein, 11 aa, Homo sapiens
DICLPRWGCLW

SEQ ID NO: 152 - protein, 11 aa, Homo sapiens
DLCLRDWGCLW

SEQ ID NO: 153 - protein, 11 aa, Homo sapiens
DICLARWGCLW

SEQ ID NO: 154 - protein, 159 aa, Artificial Sequence
  Description of Artificial Sequence Synthetic polypeptide
MGVSDVPRDLEVVAATPTSLLISWVPPSDDYGYYRITYGETGGNSPVQEFTVPIGKGTATISGLKPGVDYTITVYAVEFPWPHAGYYHRPISINYRTGSGSGSQHDEAVDANSLAEAKVLANRELDKYGVSDYYKNLINNAKTVEGVKALIDEILAALP

SEQ ID NO: 155 - protein, 115 aa, Artificial Sequence [sequence omitted]

SEQ ID NO: 156 - protein, 115 aa, Artificial Sequence [sequence omitted]

SEQ ID NO: 157 - protein, 115 aa, Artificial Sequence [sequence omitted]

SEQ ID NO: 158 - protein, 115 aa, Artificial Sequence [sequence omitted]

SEQ ID NO: 159 - protein, 115 aa, Artificial Sequence [sequence omitted]

SEQ ID NO: 160 - protein, 115 aa, Artificial Sequence
  Description of Artificial Sequence Synthetic polypeptide
EVQLVESGGGLVQPGNSLRLSCAASGFTFSSFGMSWVRQAPGKGLEWVSSISGSGSDTLYADSVKGRETISRDNAKTTLYLQMNSLRPEDTAVYYCTIGGSLSRSSQGTLVTVSS

SEQ ID NO: 161 - protein, 115 aa, Artificial Sequence
  Description of Artificial Sequence Synthetic polypeptide
EVQLVESGGGLVQPGNSLRLSCAASGFTFSSFGMSWVRQAPGKGLEWVSSISGSGSDTLYADSVKGRETISRDNAKNTLYLQMNSLRPEDTAVYYCTIGGSLSRSSQGTLVTVSS

SEQ ID NO: 162 - protein, 115 aa, Artificial Sequence [sequence omitted]

SEQ ID NO: 163 - protein, 31 aa, Artificial Sequence
  Description of Artificial Sequence Synthetic polypeptide
SCSVADWQMPPPYVVLDLPQETLEEETPGAN

SEQ ID NO: 164 - protein, 31 aa, Artificial Sequence
  Description of Artificial Sequence Synthetic polypeptide
SCCVADWQMPPPYVVLDLPQETLEEETPGAN

SEQ ID NO: 165 - protein, 26 aa, Artificial Sequence
  Description of Artificial Sequence Synthetic peptide
DWQMPPPYVVLDLPQETLEEETPGAN

SEQ ID NO: 166 - protein, 31 aa, Artificial Sequence; identical to SEQ ID NO: 164
  Description of Artificial Sequence Synthetic polypeptide

SEQ ID NO: 167 - protein, 27 aa, Artificial Sequence
  Description of Artificial Sequence Synthetic peptide
YLAMTPLIPQSKDENSDDYTTFDDVGS

SEQ ID NO: 168 - protein, 15 aa, Artificial Sequence
  Description of Artificial Sequence Synthetic peptide
ELDVCVEEAEGEAPW

SEQ ID NO: 169 - protein, 18 aa, Artificial Sequence
  Description of Artificial Sequence Synthetic peptide
ELQLEESCAEAQDGELDG

SEQ ID NO: 170 - protein, 13 aa, Artificial Sequence
  Description of Artificial Sequence Synthetic peptide
EGEVSADEEGFEN

SEQ ID NO: 171 - protein, 19 aa, Artificial Sequence
  Description of Artificial Sequence Synthetic peptide
KPTHVNVSVVMAEVDGTCY

SEQ ID NO: 172 - protein, 19 aa, Artificial Sequence
  Description of Artificial Sequence Synthetic peptide
KPTHVNVSVVMAEVDGTCY

SEQ ID NO: 173 - protein, 9 aa, Artificial Sequence
  Description of Artificial Sequence Synthetic peptide
YVTDHGPMK

SEQ ID NO: 174 - protein, 18 aa, Artificial Sequence
  Description of Artificial Sequence Synthetic peptide
PTLYNVSLVMSDTAGTCY

SEQ ID NO: 175 - protein, 31 aa, Artificial Sequence
  Description of Artificial Sequence Synthetic polypeptide
SXSVADWQMPPPYVVLDLPQETLEEETPGAN

SEQ ID NO: 176 - protein, 31 aa, Artificial Sequence
  Description of Artificial Sequence Synthetic polypeptide
SXXVADWQMPPPYVVLDLPQETLEEETPGAN

SEQ ID NO: 177 - protein, 31 aa, Artificial Sequence; identical to SEQ ID NO: 176
  Description of Artificial Sequence Synthetic polypeptide

SEQ ID NO: 178 - protein, 15 aa, Artificial Sequence
  Description of Artificial Sequence Synthetic peptide
ELDVXVEEAEGEAPW

SEQ ID NO: 179 - protein, 18 aa, Artificial Sequence
  Description of Artificial Sequence Synthetic peptide
ELQLEESXAEAQDGELDG

SEQ ID NO: 180 - protein, 19 aa, Artificial Sequence
  Description of Artificial Sequence Synthetic peptide
KPTHVNVSVVMAEVDGTXY

SEQ ID NO: 181 - protein, 19 aa, Artificial Sequence
  Description of Artificial Sequence Synthetic peptide
KPTHVNVSVVMAEVDGTXY

SEQ ID NO: 182 - protein, 18 aa, Artificial Sequence
  Description of Artificial Sequence Synthetic peptide
PTLYNVSLVMSDTAGTXY

SEQ ID NO: 183 - protein, 16 aa, Artificial Sequence
  Description of Artificial Sequence Synthetic peptide
DKTHTCPPCPAPELLG

SEQ ID NO: 184 - protein, 24 aa, Artificial Sequence
  Description of Artificial Sequence Synthetic peptide
EPKSSDKTHTCPPCPAPELLGGPS

SEQ ID NO: 185 - protein, 24 aa, Artificial Sequence
  Description of Artificial Sequence Synthetic peptide
EPKSSDKTHTCPPCPAPELLGGSS

SEQ ID NO: 186 - protein, 24 aa, Artificial Sequence
  Description of Artificial Sequence Synthetic peptide
EPKSSGSTHTCPPCPAPELLGGSS

SEQ ID NO: 187 - protein, 19 aa, Artificial Sequence
  Description of Artificial Sequence Synthetic peptide
DKTHTCPPCPAPELLGGPS

SEQ ID NO: 188 - protein, 19 aa, Artificial Sequence
  Description of Artificial Sequence Synthetic peptide
DKTHTCPPCPAPELLGGSS

SEQ ID NO: 189 - protein, 7 aa, Artificial Sequence
  Description of Artificial Sequence Synthetic peptide
AGGGGSG

SEQ ID NO: 190 - protein, 8 aa, Artificial Sequence
  Description of Artificial Sequence Synthetic peptide
AGGGGSGG

SEQ ID NO: 191 - protein, 8 aa, Artificial Sequence
  Description of Artificial Sequence Synthetic peptide
QPDEPGGS

SEQ ID NO: 192 - protein, 17 aa, Artificial Sequence
  Description of Artificial Sequence Synthetic peptide
ELQLEESAAEAQDGELD

SEQ ID NO: 193 - protein, 6 aa, Artificial Sequence
  Description of Artificial Sequence Synthetic peptide
TVAAPS

SEQ ID NO: 194 - protein, 9 aa, Artificial Sequence
  Description of Artificial Sequence Synthetic peptide
QPDEPGGSG

SEQ ID NO: 195 - protein, 18 aa, Artificial Sequence
  Description of Artificial Sequence Synthetic peptide
ELQLEESAAEAQDGELDG

SEQ ID NO: 196 - protein, 7 aa, Artificial Sequence
  Description of Artificial Sequence Synthetic peptide
TVAAPSG

SEQ ID NO: 197 - protein, 6 aa, Artificial Sequence
  Description of Artificial Sequence Synthetic peptide
GSGSGS

SEQ ID NO: 198 - protein, 686 aa, Artificial Sequence
  Description of Artificial Sequence Synthetic polypeptide
VSDVPRDLEVVAATPTSLLISWGHYPLHVRYYRITYGETGGNSPVQEFTVPSRKYTATISGLKPGVDYTITVYAVTYYKEANYREIPISINYRTEGSGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 199 - protein, 686 aa, Artificial Sequence
  Description of Artificial Sequence Synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGSGSVSDVPRDLEVVAATPTSLLISWGHYPLHVRYYRITYGETGGNSPVQEFTVPSRKYTATISGLKPGVDYTITVYAVTYYKEANYREIPISINYRTE

SEQ ID NO: 200 - protein, 687 aa, Artificial Sequence
  Description of Artificial Sequence Synthetic polypeptide
VSDVPRDLEVVAATPTSLLISWVPPSDDYGYYRITYGETGGNSPVQEFTVPIGKGTATISGLKPGVDYTITVYAVEFPWPHAGYYHRPISINYRTEGSGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 201 - protein, 687 aa, Artificial Sequence
  Description of Artificial Sequence Synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGSGSVSDVPRDLEVVAATPTSLLISWVPPSDDYGYYRITYGETGGNSPVQEFTVPIGKGTATISGLKPGVDYTITVYAVEFPWPHAGYYHRPISINYRTE

SEQ ID NO: 202 - protein, 805 aa, Artificial Sequence
  Description of Artificial Sequence Synthetic polypeptide
VSDVPRDLEVVAATPTSLLISWSARLKVARYYRITYGETGGNSPVQEFTVPKNVYTATISGLKPGVDYTITVYAVTRFRDYQPISINYRTEIDKGSGSGSGSGSGSGSGSGSGSVSDVPRDLEVVAATPTSLLISWWAPVDRYQYYRITYGETGGNSPVQEFTVPRDVYTATISGLKPGVDYTITVYAVTDYKPHADGPHTYHESPISINYRTEGSGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 203 - protein, 805 aa, Artificial Sequence
  Description of Artificial Sequence Synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGSGSVSDVPRDLEVVAATPTSLLISWSARLKVARYYRITYGETGGNSPVQEFTVPKNVYTATISGLKPGVDYTITVYAVTRFRDYQPISINYRTEIDKGSGSGSGSGSGSGSGSGSGSVSDVPRDLEVVAATPTSLLISWWAPVDRYQYYRITYGETGGNSPVQEFTVPRDVYTATISGLKPGVDYTITVYAVTDYKPHADGPHTYHESPISINYRTE

SEQ ID NO: 204 - protein, 788 aa, Artificial Sequence
  Description of Artificial Sequence Synthetic polypeptide
VSDVPRDLEVVAATPTSLLISWSARLKVARYYRITYGETGGNSPVQEFTVPKNVYTATISGLKPGVDYTITVYAVTRFRDYQPISINYRTEGSGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGSGSVSDVPRDLEVVAATPTSLLISWWAPVDRYQYYRITYGETGGNSPVQEFTVPRDVYTATISGLKPGVDYTITVYAVTDYKPHADGPHTYHESPISINYRTE

SEQ ID NO: 205 - protein, 788 aa, Artificial Sequence
  Description of Artificial Sequence Synthetic polypeptide
VSDVPRDLEVVAATPTSLLISWWAPVDRYQYYRITYGETGGNSPVQEFTVPRDVYTATISGLKPGVDYTITVYAVTDYKPHADGPHTYHESPISINYRTEGSGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGSGSVSDVPRDLEVVAATPTSLLISWSARLKVARYYRITYGETGGNSPVQEFTVPKNVYTATISGLKPGVDYTITVYAVTRFRDYQPISINYRTE

SEQ ID NO: 206 - DNA, 6 bp, Artificial Sequence
  Description of Artificial Sequence Synthetic oligonucleotide
cncaat

SEQ ID NO: 207 - DNA, 6 bp, Artificial Sequence
  Description of Artificial Sequence Synthetic oligonucleotide
aataaa

SEQ ID NO: 208 - protein, 165 aa, Artificial Sequence
  Description of Artificial Sequence Synthetic polypeptide
MGVSDVPRDLEVVAATPTSLLISWVPPSDDYGYYRITYGETGGNSPVQEFTVPIGKGTATISGLKPGVDYTITVYAVEFPWPHAGYYHRPISINYRTGSGSGSQHDEAVDANSLAEAKVLANRELDKYGVSDYYKNLINNAKTVEGVKALIDEILAALPHHHHHH

SEQ ID NO: 209 - protein, 168 aa, Artificial Sequence
  Description of Artificial Sequence Synthetic polypeptide
MGQHDEAVDANSLAEAKVLANRELDKYGVSDYYKNLINNAKTVEGVKALIDEILAALPGSGSGSVSDVPRDLEVVAATPTSLLISWRHPHFPTRYYRITYGETGGNSPVQEFTVPLQPPTATISGLKPGVDYTITVYAVTDGRNGRLLSIPISINYRTGSGSHHHHHH

SEQ ID NO: 210 - protein, 51 aa, Streptococcus sp.
VDANSLAEAKVLANRELDKYGVSDYYKNLINNAKTVEGVKALIDEILAALP

SEQ ID NO: 211 - protein, 5 aa, Homo sapiens
EIDKP

SEQ ID NO: 212 - protein, 6 aa, Homo sapiens
EIDKPS

SEQ ID NO: 213 - protein, 3 aa, Artificial Sequence
  Description of Artificial Sequence Synthetic peptide
MGL

SEQ ID NO: 214 - protein, 4 aa, Artificial Sequence
  Description of Artificial Sequence Synthetic peptide
GSGS

SEQ ID NO: 215 - protein, 8 aa, Artificial Sequence
  Description of Artificial Sequence Synthetic peptide
GSGSGSGS

SEQ ID NO: 216 - protein, 14 aa, Artificial Sequence
  Description of Artificial Sequence Synthetic peptide
GSGSGSGSGSGSGS

SEQ ID NO: 217 - protein, 16 aa, Artificial Sequence
  Description of Artificial Sequence Synthetic peptide
GSGSGSGSGSGSGSGS

SEQ ID NO: 218 - protein, 18 aa, Artificial Sequence
  Description of Artificial Sequence Synthetic peptide
GSGSGSGSGSGSGSGSGS

SEQ ID NO: 219 - protein, 5 aa, Artificial Sequence
  Description of Artificial Sequence Synthetic peptide
GGGGS

SEQ ID NO: 220 - protein, 10 aa, Artificial Sequence
  Description of Artificial Sequence Synthetic peptide
GGGGSGGGGS

SEQ ID NO: 221 - protein, 15 aa, Artificial Sequence
  Description of Artificial Sequence Synthetic peptide
GGGGSGGGGSGGGGS

SEQ ID NO: 222 - protein, 6 aa, Artificial Sequence
  Description of Artificial Sequence Synthetic peptide
EDEDED

(19) United States
(12) Patent Application Publication
(10) Pub. No.: US 2016/0152686 A1
(43) Pub. Date: Jun. 2, 2016

(54) FIBRONECTIN BASED SCAFFOLD DOMAINS LINKED TO SERUM ALBUMIN OR MOIETY BINDING THERETO

(71) Applicant: BRISTOL-MYERS SQUIBB COMPANY, Princeton, NJ (US)

(72) Inventors: Ray Camphausen, Wayland, MA (US); Tracy S. Mitchell, Andover, MA (US)

(21) Appl. No.: 14/774,229

(22) PCT Filed: Mar. 12, 2014
(86) PCT No.: PCT/US14/24350

§ 371 Date: Sep. 10, 2015

Related U.S. Application Data
(60) Provisional application No. 61/779,086, filed on Mar. 13, 2013.

(51) Int. Cl. C07K 14/765; C07K 14/78

(52) U.S. Cl. CPC C07K 14/765; C07K 14/78; A61K 38/00

(57) ABSTRACT

Provided herein are fusion proteins comprising a first moiety comprising a fibronectin based scaffold and a second moiety comprising serum albumin or a biologically active fragment thereof or a serum albumin binding domain or a biologically active fragment thereof.

### CROSS-REFERENCE TO RELATED APPLICATIONS

This application claims the benefit of U.S. Provisional application No. 61/779,086, filed Mar. 13, 2013, the contents of which are specifically incorporated by reference herein.

### BACKGROUND

The utility of many therapeutics, particularly biologicals such as peptides, polypeptides and polynucleotides, suffer from inadequate serum half-lives. This necessitates the administration of such therapeutics at high frequencies and/or higher doses, or the use of sustained release formulations, in order to maintain the serum levels necessary for therapeutic effects. Frequent systemic administration of drugs is associated with considerable negative side effects. For example, frequent systemic injections represent a considerable discomfort to the subject, and pose a high risk of administration related infections, and may require hospitalization or frequent visits to the hospital, in particular when the therapeutic is to be administered intravenously. Moreover, in long term treatments daily intravenous injections can also lead to considerable side effects of tissue scarring and vascular pathologies caused by the repeated puncturing of vessels. Similar problems are known for all frequent systemic administrations of therapeutics, such as, for example, the administration of insulin to diabetics, or interferon drugs in patients suffering from multiple sclerosis. All these factors lead to a decrease in patient compliance and increased costs for the health system.

### SUMMARY

Provided herein are stabilized target binding proteins (or fusion proteins) that specifically bind to a target. A protein may comprise (i) an amino acid sequence encoding a fibronectin based scaffold (FBS) moiety that specifically binds to a target; and (ii) an amino acid sequence encoding a human serum albumin (HSA) moiety, wherein the target binding protein has one or more improved properties relative to the FBS protein that is not linked to HSA. The one or more improved properties may be selected from the group consisting of improved pharmacokinetics, increased shelf-life, increased solubility, increased affinity for the target and increased biological activity. The FBS moiety may comprise a ¹⁰Fn3 moiety. The ¹⁰Fn3 domain may comprise 1-30 amino acid changes relative to a wild type human ¹⁰Fn3 moiety comprising any one of SEQ ID NOs: 1-16. The HSA moiety may be a wild type HSA moiety. The amino acid sequence encoding the FBS moiety and the amino acid sequence encoding the HSA moiety may be arranged in an amino- to carboxy-terminal order or in a carboxy- to amino-terminal order. The amino acid sequence encoding the FBS moiety and the amino acid sequence encoding the HSA moiety may be connected through a linker consisting of at least one amino acid, e.g., a linker consisting of 1-25 amino acids. A linker may be a GS linker, e.g., a (GS)₃ linker.

Exemplary target binding proteins comprise (i) an FBS moiety comprising an amino acid sequence that is at least about 60% identical to any one of SEQ ID NOs: 1-16; and (ii) an HSA moiety comprising an amino acid sequence that is at least about 90% identical to SEQ ID NO: 102, wherein the target binding protein binds specifically to a target with a K_{d} of less than 500 nM, and wherein the HSA moiety improves at least one property of the FBS moiety relative to a protein consisting of the unmodified FBS moiety. Exemplary target binding proteins may also comprise (i) an FBS moiety comprising an amino acid sequence that is at least about 60% identical to any one of SEQ ID NOs: 1-16; (ii) an HSA moiety comprising an amino acid sequence that is at least about 90% identical to SEQ ID NO: 102; and (iii) a linker that covalently links the FBS moiety to the HSA moiety, wherein the target binding protein binds specifically to a target with a K_{d} of less than 500 nM, and wherein the HSA moiety improves at least one property of the FBS moiety relative to a protein consisting of the unmodified FBS moiety. A linker may be 1-10 amino acids long. A linker may comprise, consist of, or consist essentially of (GS)₃. The HSA moiety may comprise, consist of, or consist essentially of SEQ ID NO: 102.

Target binding proteins (e.g., fusion proteins) may also comprise (i) an amino acid sequence encoding an FBS moiety that specifically binds to a target; and (ii) an amino acid sequence encoding an albumin binding domain (ABD) moiety, wherein the target binding protein HSA one or more improved properties relative to the FBS protein that is not linked to ABD. The one or more improved properties may be selected from the group consisting of improved pharmacokinetics, increased shelf-life, increased solubility, increased affinity for the target and increased biological activity. The FBS moiety may comprise a ¹⁰Fn3 moiety. The ¹⁰Fn3 domain may comprise 1-30 amino acid changes relative to a wild type human ¹⁰Fn3 moiety comprising any one of SEQ ID NOs: 1-16. The ABD moiety may be a wild type ABD moiety. The amino acid sequence encoding the FBS moiety and the amino acid sequence encoding the ABD moiety may be arranged in an amino- to carboxy-terminal order or in a carboxy- to amino-terminal order. The amino acid sequence encoding the FBS moiety and the amino acid sequence encoding the ABD moiety may be connected through a linker consisting of at least one amino acid. The linker may consist of 1-25 amino acids. The linker may be a GS linker, e.g., a (GS)₃ linker.

In exemplary embodiments, a target binding protein comprises (i) an FBS moiety comprising an amino acid sequence that is at least about 60% identical to any one of SEQ ID NOs: 1-16; and (ii) an ABD moiety comprising an amino acid sequence that is at least about 90% identical to SEQ ID NO: 112, wherein the target binding protein binds specifically to a target with a K_{d} of less than 500 nM, and wherein the ABD moiety improves at least one property of the FBS moiety relative to a protein consisting of the unmodified FBS moiety. Exemplary target binding proteins may also comprise (i) an FBS moiety comprising an amino acid sequence that is at least about 60% identical to any one of SEQ ID NOs: 1-16; (ii) an ABD moiety comprising an amino acid sequence that is at least about 90% identical to SEQ ID NO: 112; and (iii) a linker that covalently links the FBS moiety to the ABD moiety, wherein the target binding protein binds specifically to a target with a K_{d} of less than 500 nM, and wherein the ABD moiety improves at least one property of the FBS moiety relative to a protein consisting of the unmodified FBS moiety. The linker may be 1-10 amino acids long. The linker may comprise, consist of, or consist essentially of (GS)₃. The ABD moiety may comprise, consist of, or consist essentially of any one of SEQ ID NOs: 103-112. A target binding protein may have a half-life in Cynomolgus monkeys that is at least 10 fold as long relative to that of the FBS protein that is not linked to an ABD moiety. A protein may have a half-life in Cynomolgus monkeys of at least 80 hours. The biological activity of the FBS moiety linked to ABD may be at least as strong as that of the FBS moiety when it is not linked to an ABD moiety.

Also provided herein are pharmaceutical compositions comprising a target binding protein (or fusion protein) and a pharmaceutically acceptable carrier. Further provided are nucleic acids encoding a target binding protein (or fusion protein) described herein.

### BRIEF DESCRIPTION OF THE DRAWINGS

FIGS. 1A-D shows the amino acid sequences of 8 exemplary fusion proteins (or target binding proteins) comprising one or more FBS moieties covalently linked to an HSA moiety (SEQ ID NOs: 198-205). The amino acid sequences of the FBS moieties are underlined and those of the linkers are shown in italics.

FIG. 2 shows the HSA preprotein (SEQ ID NO: 101) and the mature HSA protein (SEQ ID NO: 102).

FIGS. 3A-D shows exemplary mutants of ABD (SEQ ID NO: 113-150).

FIGS. 4A and B show graphs of the SEC and DSC analyses of ABD-(GS)3-C7FL-His6, respectively.

FIG. 5 shows the results of a BaF3 proliferation assay with ABD-(GS)3-C7FL-His6.

### DETAILED DESCRIPTION

Provided herein are fusion proteins comprising one or more FBS moieties linked to a human serum albumin (HSA) or an HSA binding moiety.

### DEFINITIONS

An “amino acid residue” is the remaining portion of an amino acid after a water molecule has been lost (an H+ from the nitrogenous side and an OH— from the carboxylic side) in the formation of a peptide bond.

As used herein, a “¹⁰Fn3 domain” or “¹⁰Fn3 moiety” refers to wild-type ¹⁰Fn3 and biologically active variants thereof, e.g., biologically active variants that specifically bind to a target, such as a target protein. A wild-type human ¹⁰Fn3 domain may comprise one of the amino acid sequences set forth in SEQ ID NO: 1-8, 9, 11, 13, 15, 17, 19, 21 or 23). Biologically active variants of a wild-type human ¹⁰Fn3 domain include ¹⁰Fn3 domains that comprise at least, at most or about 1, 2, 3, 4, 5, 6, 7, 8, 9, 10, 11, 12, 13, 14, 15, 16, 17, 18, 19, 20, 21, 22, 23, 24, 25, 30, 35, 40 or 45 amino acid changes, i.e., substitutions, additions or deletions, relative to a ¹⁰Fn3 domain comprising any one of SEQ ID NOs: 1-8, 9, 11, 13, 15, 17, 19, 21 or 23. A biologically active variant of a wild-type ¹⁰Fn3 domain may also comprise, or comprise at most, 1-3, 1-5, 1-10, 1-15, 1-10, 1-25, 1-30, 1-35, 1-40 or 1-45 amino acid changes relative to a ¹⁰Fn3 domain comprising any one of SEQ ID NOs: 1-8, 9, 11, 13, 15, 17, 19, 21 or 23. In certain embodiments, a biologically active variant of a wild-type ¹⁰Fn3 domain does not comprise more than 1, 2, 3, 4, 5, 6, 7, 8, 9, 10, 11, 12, 13, 14, 15, 16, 17, 18, 19, 20, 21, 22, 23, 24, 25, 30, 35, 40 or 45 amino acid changes, i.e., substitutions, additions or deletions, relative to an ¹⁰Fn3 domain comprising any one of SEQ ID NOs: 1-8, 9, 11, 13, 15, 17, 19, 21 or 23 Amino acid changes may be in a loop region, in a strand or in the N-terminal or C-terminal region. Exemplary degenerate ¹⁰Fn3 amino acid sequences allowing for amino acid changes in the loop regions are provided herein as SEQ ID NOs: 25-48.

By “polypeptide” is meant any sequence of two or more amino acids, regardless of length, post-translation modification, or function. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

A “region” of a ¹⁰Fn3 domain (or moiety) as used herein refers to either a loop (AB, BC, CD, DE, EF and FG), a β-strand (A, B, C, D, E, F and G), the N-terminus (corresponding to amino acid residues 1-7 of SEQ ID NO: 1), or the C-terminus (corresponding to amino acid residues 93-101 of SEQ ID NO: 9) of a ¹⁰Fn3 domain, e.g., having SEQ ID NO: 9.

A “north pole loop” of a ¹⁰Fn3 domain (or moiety) refers to any one of the BC, DE and FG loops of a ¹⁰Fn3 domain.

A “south pole loop” of a ¹⁰Fn3 domain (or moiety) refers to any one of the AB, CD and EF loops of a ¹⁰Fn3 domain.

A “scaffold region” refers to any non-loop region of a human ¹⁰Fn3 domain. The scaffold region includes the A, B, C, D, E, F and G β-strands as well as the N-terminal region (amino acids corresponding to residues 1-7 of SEQ ID NO: 1 or 9) and the C-terminal region (amino acids corresponding to residues 93-101 of SEQ ID NO: 9).

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST^{SM}, BLAST^{SM}-2, ALIGN, ALIGN-2 or Megalign (DNASTAR®) software. Those skilled in the art can determine appropriate parameters for measuring alignment, including any algorithms needed to achieve maximal alignment over the full-length of the sequences being compared.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by a sequence alignment program, such as BLAST^{SM}, BLAST^{SM}-2, ALIGN, ALIGN-2 or Megalign (DNASTAR®), in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, an amino acid residue in a polypeptide is considered to “contribute to binding” a target if (1) any of the non-hydrogen atoms of the residue's side chain or main chain is found to be within five angstroms of any atom of the binding target based on an experimentally determined three-dimensional structure of the complex, and/or (2) mutation of the residue to its equivalent in wild-type ¹⁰Fn3 (e.g., SEQ ID NO: 1), to alanine, or to a residue having a similarly sized or smaller side chain than the residue in question, leads to a measured increase of the equilibrium dissociation constant to the target (e.g., an increase in the kₒₙ).

“Moiety” refers to a portion of a protein. For example, a fusion protein may comprise several moieties. In one embodiment, a fusion protein comprises an FBS moiety and an HSA moiety or an HSA binding moiety. “HSA moiety” refers to an HSA portion of a larger protein. “HSA binding moiety” refers to an HSA binding portion of a larger protein. An HSA moiety can have the same length or same amino acid sequence as that of wild-type mature HSA (having, e.g., SEQ ID NO: 102) or that of wild-type HSA preprotein comprising the HSA signal sequence (having, e.g., SEQ ID NO: 101), or it can comprise or consist of variants thereof, such as fragments thereof. An HSA binding moiety can have the same length or same amino acid sequence as that of a wild-type HSA binding protein or it can comprise or consist of variants thereof, e.g., binding fragments thereof.

The serum or plasma “half-life” of a polypeptide can generally be defined as the time taken for the serum concentration of the polypeptide to be reduced by 50%, in vivo, for example due to degradation of the polypeptide and/or clearance or sequestration of the polypeptide by natural mechanisms. The half-life can be determined in any manner known per se, such as by pharmacokinetic analysis. Suitable techniques will be clear to the person skilled in the art, and may, for example, generally involve the steps of administering a suitable dose of a polypeptide to a primate; collecting blood samples or other samples from said primate at regular intervals; determining the level or concentration of the polypeptide in said blood sample; and calculating, from (a plot of) the data thus obtained, the time until the level or concentration of the polypeptide has been reduced by 50% compared to the initial level upon dosing. Methods for determining half-life may be found, for example, in Kenneth et al., Chemical Stability of Pharmaceuticals: A Handbook for Pharmacists (1986); Peters et al., Pharmacokinete Analysis: A Practical Approach (1996); and Gibaldi, M. et al., Pharmacokinetics, Second Rev. Edition, Marcel Dekker (1982).

Serum half-life can be expressed using parameters such as the t½-alpha, t½-beta and the area under the curve (AUC). An “increase in half-life” refers to an increase in any one of these parameters, any two of these parameters, or in all three these parameters. In certain embodiments, an increase in half-life refers to an increase in the t½-beta, either with or without an increase in the t½-alpha and/or the AUC or both.

“Shelf-life” of a pharmaceutical product, e.g., a fusion protein comprising an FBS moiety and an HSA moiety, is the length of time the product is stored before decomposition occurs. For example, shelf-life may be defined as the time for decomposition of 0.1%, 0.5%, 1%, 5%, or 10% of the product.

### Overview

Provided herein are fusion proteins comprising at least one fibronectin based scaffold (FBS) moiety and a human serum albumin (HSA) moiety or an HSA binding moiety, which proteins have favorable properties relative to the FBS that is not linked to HSA or the HSA binding moiety (i.e., the “unmodified” FBS), e.g., an extended half-life in serum. The application is based at least in part on the discovery that linking a ¹⁰Fn3 molecule, which binds specifically to a given target, to an HSA binding moiety consisting of an Albumin Binding Domain (ABD) of streptococcal protein G, increases at least one characteristic of the ¹⁰Fn3 molecule, including its pharmacokinetic properties, relative to the unmodified ¹⁰Fn3 molecule.

The FBS polypeptides described herein may be designed to bind to any target of interest. In exemplary embodiments, the target is an antigen, a polypeptide or a therapeutic protein target of interest. Exemplary therapeutically desirable targets, include, for example, tumor necrosis factor alpha (TNF-alpha), VEGFR2, PCSK9, IL-23, EGFR and IGF1R.

### Fibronectin Based Scaffolds

General Structure

As used herein, a “fibronectin based scaffold” or “FBS” protein or moiety refers to proteins or moieties that are based on a fibronectin type III (“Fn3”) repeat. Fn3 is a small (about 10 kDa) domain that has the structure of an immunoglobulin (Ig) fold (i.e., an Ig-like β-sandwich structure, consisting of seven β-strands and six loops). Fibronectin has 18 Fn3 repeats, and while the sequence homology between the repeats is low, they all share a high similarity in tertiary structure. Fn3 domains are also present in many proteins other than fibronectin, such as adhesion molecules, cell surface molecules, e.g., cytokine receptors, and carbohydrate binding domains. For reviews see Bork et al., Proc. Natl. Acad. Sci. USA, 89(19):8990-8994 (1992); Bork et al., J. Mol. Biol., 242(4):309-320 (1994); Campbell et al., Structure, 2(5):333-337 (1994); Harpez et al., J. Mol. Biol., 238(4):528-539 (1994)). The term “FBS” protein or moiety is intended to include scaffolds based on Fn3 domains from these other proteins (i.e., non fibronectin molecules).

An Fn3 domain is small, monomeric, soluble, and stable. It lacks disulfide bonds and, therefore, is stable under reducing conditions. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel β-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”).

The loops in Fn3 molecules are structurally similar to complementary determining regions (CDRs) of antibodies, and when altered, may be involved in binding of the Fn3 molecule to a target, e.g., a target protein. Other regions of Fn3 molecules, such as the beta or beta-like strands and N-terminal or C-terminal regions, when altered, may also be involved in binding to a target. Any or all of loops AB, BC, CD, DE, EF and FG may participate in binding to a target. Any of the beta or beta-like strands may be involved in binding to a target. Fn3 domains may also bind to a target through one or more loops and one or more beta or beta-like strands. Binding may also require the N-terminal or C-terminal regions. An FBS domain for use in a fusion protein may comprise all loops, all beta or beta-like strands, or only a portion of them, wherein certain loops and/or beta or beta-like strands and/or N- or C-terminal regions are modified (or altered), provided that the FBS domain preferably binds specifically to a target. For example, an FBS domain may comprise 1, 2, 3, 4, 5 or 6 loops, 1, 2, 3, 4, 5, 6, 7, or 8 beta strands, and optionally an N-terminal and/or C-terminal region, wherein one or more loops, one or more beta strands, the N-terminal region and/or the C-terminal regions are modified relative to the wild-type FBS domain.

In exemplary embodiments, ligand (or target) binding FBS moieties described herein are based on the tenth fibronectin type III domain, i.e., the tenth module of Fn3 (¹⁰Fn3). The amino acid sequence of a wild-type human ¹⁰Fn3 moiety is as follows:

(SEQ ID NO: 1)

VSDVPRDLEVVAATPTSLLISWDAPAVTVRYYRITYGETGGNSPVQEFT
VPGSKSTATISGLKPGVDYTITVYAVTGRGDSPASSKPISINYRT

(the AB, CD and EF loops are underlined; the BC, FG, and DE loops are emphasized in bold; the β-strands are located between or adjacent to each of the loop regions; and the N-terminal region is shown in italics). The last two amino acid residues of SEQ ID NO: 1 are a portion of a C-terminal region.

Wild-type human ¹⁰Fn3 molecules also include those lacking the N-terminal region or a portion thereof. For example, a wild-type ¹⁰Fn3 molecule may comprise SEQ ID NO: 1, wherein amino acid residues 1, 1-2, 1-3, 1-4, 1-5, 1-6 or 1-7 are deleted (SEQ ID NOs: 2-8, respectively). Table 1 shows the amino acid sequence of these wild-type human ¹⁰Fn3 moieties:

TABLE 1
Amino acid sequences of wild-type human ¹⁰Fn3 molecules with
various N-terminal regions

| | N-terminal | | |
|---|---|---|---|
| Version | region | Wild-type human 10Fn3 Core Domain | Full length |
| 1 | VSDVPRD | LEVVAATPTSLLISWDAPAVTVRYYRITY | SEQ ID NO: 1 |
| | (SEQ ID NO: | GETGGNSPVQEFTVPGSKSTATISGLKPG | |
| | 51 | VDYTITVYAVTGRGDSPASSKPISINYRT | |
| | | (SEQ ID NO: 49) | |
| 2 | SDVPRD | LEVVAATPTSLLISWDAPAVTVRYYRITY | SEQ ID NO: 2 |
| | (SEQ ID NO: | GETGGNSPVQEFTVPGSKSTATISGLKPG | |
| | 52) | VDYTITVYAVTGRGDSPASSKPISINYRT | |
| | | (SEQ ID NO: 49) | |
| 3 | DVPRD | LEVVAATPTSLLISWDAPAVTVRYYRITY | SEQ ID NO: 3 |
| | (SEQ ID NO: | GETGGNSPVQEFTVPGSKSTATISGLKPG | |
| | 53) | VDYTTTVYAVTGRGDSPASSKPISINYRT | |
| | | (SEQ ID NO: 49) | |
| 4 | VPRD | LEVVAATPTSLLISWDAPAVTVRYYRITY | SEQ ID NO: 4 |
| | (SEQ ID NO: | GETGGNSPVQEFTVPGSKSTATISGLKPG | |
| | 54) | VDYTITVYAVTGRGDSPASSKPISTNYRT | |
| | | (SEQ ID NO: 49) | |
| 5 | PRD | LEVVAATPTSLLISWDAPAVTVRYYRITY | SEQ ID NO: 5 |
| | | GETGGNSPVQEFTVPGSKSTATISGLKPG | |
| | | VDYTITVYAVTGRGDSPASSKPISINYRT | |
| | | (SEQ ID NO: 49) | |
| 6 | RD | LEVVAATPTSLLISWDAPAVTVRYYRITY | SEQ ID NO: 6 |
| | | GETGGNSPVQEFTVPGSKSTATISGLKPG | |
| | | VDYTITVYAVTGRGDSPASSKPISINYRT | |
| | | (SEQ ID NO: 49) | |
| 7 | D | LEVVAATPTSLLISWDAPAVTVRYYRITY | SEQ ID NO: 7 |
| | | GETGGNSPVQEFTVPGSKSTATISGLKPG | |
| | | VDYTTTVYAVTGRGDSPASSKPTSINYRT | |
| | | (SEQ ID NO: 49) | |
| 8 | — | LEVVAATPTSLLISWDAPAVTVRYYRITY | SEQ ID NO: 8 |
| | | GETGGNSPVQEFTVPGSKSTATISGLKPG | |
| | | VDYTITVYAVTGRGDSPASSKPISINYRT | |
| | | (SEQ ID NO: 49) | |

Wild-type human ¹⁰Fn3 moieties also include those ¹⁰Fn3 moieties comprising one or more of the naturally occurring amino acids adjacent to those in SEQ ID NO: 1 in the human fibronectin molecule. For example, a wild-type ¹⁰Fn3 molecules may comprise SEQ ID NO: 1 or any other molecule shown in Table 1, and 1, 2, 3, 4, 5, 6 or 7 additional amino acids from the natural tail (or C-terminal region) of human ¹⁰Fn3, i.e., EIDKPSQ (SEQ ID NO: 55). It has been previously shown that changing this sequence to EIEKPSQ (SEQ ID NO: 56) may stabilize a ¹⁰Fn3 molecule. Table 2 shows the amino acid sequence of exemplary wild-type human ¹⁰Fn3 moieties comprising the naturally occurring tail portion having SEQ ID NO: 55 or its derivative having SEQ ID NO: 56. FBS moieties may be derived from (or based on) any of these molecules, as well as molecules comprising a portion of the C-terminal tail consisting of SEQ ID NO: 55 or 56, e.g., comprising E, EI, EID, EIDK, EIDKP, EIDKPS, EIE, EIEK, EIEKP or EIEKPS covalently linked to a moiety comprising SEQ ID NO: 49.

TABLE 2
Amino acid sequences of wild-type (or mutated) human ¹⁰Fn3 molecules
with various N-terminal regions and either of two C-terminal regions

| | N-terminal | Wild-type human ¹⁰Fn3 Core | C-terminal | |
|---|---|---|---|---|
| Version | region | Domain | region | Full length |
| 1 | VSDVPRD | LEVVAATPTSLLISWDAP | EIDKPSQ | SEQ ID NO: 9 |
| | (SEQ ID NO: | AVTVRYYRITYGETGGN | (SEQ ID NO: | |
| | 51) | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 10 |
| | | (SEQ ID NO: 49) | 56) | |
| 2 | SDVPRD | LEVVAATPTSLLISWDAP | EIDKPSQ | SEQ ID NO: |
| | (SEQ ID NO: | AVTVRYYRITYGETGGN | (SEQ ID NO: | 11 |
| | 52) | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 12 |
| | | (SEQ ID NO: 49) | 56) | |
| 3 | DVPRD | LEVVAATPTSLLISWDAP | EIDKPSQ | SEQ ID NO: |
| | (SEQ ID NO: | AVTVRYYRITYGETGGN | (SEQ ID NO: | 13 |
| | 53) | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 14 |
| | | (SEQ ID NO: 49) | 56) | |
| 4 | VPRD | LEVVAATPTSLLISWDAP | EIDKPSQ | SEQ ID NO: |
| | (SEQ ID NO: | AVTVRYYRITYGETGGN | (SEQ ID NO: | 15 |
| | 54) | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 16 |
| | | (SEQ ID NO: 49) | 56) | |
| 5 | PRD | LEVVAATPTSLLISWDAP | EIDKPSQ | SEQ ID NO: |
| | | AVTVRYYRITYGETGGN | (SEQ ID NO: | 17 |
| | | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 18 |
| | | (SEQ ID NO: 49) | 56) | |
| 6 | RD | LEVVAATPTSLLISWDAP | EIDKPSQ | SEQ ID NO: |
| | | AVTVRYYRITYGETGGN | (SEQ ID NO: | 19 |
| | | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 20 |
| | | (SEQ ID NO: 49) | 56) | |
| 7 | D | LEVVAATPTSLLISWDAP | EIDKPSQ | SEQ ID NO: |
| | | AVTVRYYRITYGETGGN | (SEQ ID NO: | 21 |
| | | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 22 |
| | | (SEQ ID NO: 49) | 56) | |
| 8 | — | LEVVAATPTSLLISWDAP | EIDKPSQ | SEQ ID NO: |
| | | AVTVRYYRITYGETGGN | (SEQ ID NO: | 23 |
| | | SPVQEFTVPGSKSTATISG | 55) | |
| | | LKPGVDYTITVYAVTGR | EIEKPSQ | SEQ ID NO: |
| | | GDSPASSKPISINYRT | (SEQ ID NO: | 24 |
| | | (SEQ ID NO: 49) | 56) | |

In some embodiments, the AB loop corresponds to residues 14-17, the BC loop corresponds to residues 23-31, the CD loop corresponds to residues 37-47, the DE loop corresponds to residues 51-56, the EF loop corresponds to residues 63-67, and the FG loop corresponds to residues 75-87 of SEQ ID NO: 1 or 9. The BC, DE and FG loops align along one face of the molecule, i.e., the “north pole”, and the AB, CD and EF loops align along the opposite face of the molecule, i.e., the “south pole”. In SEQ ID NO: 1 or 9, β-strand A corresponds to residues 8-13, β-strand B corresponds to residues 18-22, β-strand C corresponds to residues 32-36, beta strand D corresponds to residues 48-50, β-strand E corresponds to residues 57-62, β-strand F corresponds to residues 68-74, and β-strand G corresponds to residues 88-92. The β-strands are connected to each other through the corresponding loop, e.g., strands A and B are connected via loop AB in the formation β-strand A, loop AB, β-strand B, etc.

An example of FBS proteins that are based on human ¹⁰Fn3 domains are adnectins (Adnexus, a wholly owned subsidiary of Bristol-Myers Squibb). Adnectins are ¹⁰Fn3 molecules in which CDR-like loop regions, β-strands, N-terminal and/or C-terminal regions of a ¹⁰Fn3 domain has been modified to evolve a protein capable of binding to a compound of interest. For example, U.S. Pat. No. 7,115,396 describes ¹⁰Fn3 domain proteins wherein alterations to the BC, DE, and FG loops result in high affinity TNFα binders. U.S. Pat. No. 7,858,739 describes Fn3 domain proteins wherein alterations to the BC, DE, and FG loops result in high affinity VEGFR2 binders.

In certain embodiments, the FBS moiety comprises a ¹⁰Fn3 domain that is defined generally by the following degenerate sequence:

(SEQ ID NO: 25)

VSDVPRDLEVVAA(X)ᵤLLISW(X)ᵥYRITY(X)_{w}FTV(X)ₓATISG
L(X)_{y}YTITVYA(X)_{z}ISINYRT,

or by a sequence selected from the group of SEQ ID NO: 26-32, which sequences are identical to SEQ ID NO: 25, except that they are lacking 1, 2, 3, 4, 5, 6 or 7 N-terminal amino acids, respectively. The amino acid sequences SEQ ID NOs: 25-32 are set forth in Table 3.

TABLE 3
Amino acid sequences of degenerate wild-type human
¹⁰Fn3 molecules with various N-terminal regions

| | N-terminal | Degenerate wild-type human ¹⁰Fn3 Core | |
|---|---|---|---|
| Version | region | Domain | Full length |
| 1 | VSDVPRD | LEVVAA(X)ᵤLLISW(X)ᵥYRITY(X)_{w}FTV | SEQ ID NO: 25 |
| | (SEQ ID NO: | (X)ₓATISGL(X)_{y}YTITVYA(X)_{z}ISINYRT | |
| | 51) | (SEQ ID NO: 50) | |
| 2 | SDVPRD | LEVVAA(X)ᵤLLISW(X)ᵥYRITY(X)_{w}FTV | SEQ ID NO: 26 |
| | (SEQ ID NO: | (X)ₓATISGL(X)_{y}YTITVYA(X)_{z}ISINYRT | |
| | 52) | (SEQ ID NO: 50) | |
| 3 | DVPRD | LEVVAA(X)ᵤLLISW(X)ᵥYRITY(X)_{w}FTV | SEQ ID NO: 27 |
| | (SEQ ID NO: | (X)ₓATISGL(X)_{y}YTITVYA(X)_{z}ISINYRT | |
| | 53) | (SEQ ID NO: 50) | |
| 4 | VPRD | LEVVAA(X)ᵤLLISW(X)ᵥYRITY(X)_{w}FTV | SEQ ID NO: 28 |
| | (SEQ ID NO: | (X)ₓATISGL(X)_{y}YTITVYA(X)_{z}ISINYRT | |
| | 54) | (SEQ ID NO: 50) | |
| 5 | PRD | LEVVAA(X)ᵤLLISW(X)ᵥYRITY(X)_{w}FTV | SEQ ID NO: 29 |
| | | (X)ₓATISGL(X)_{y}YTITVYA(X)_{z}ISINYRT | |
| | | (SEQ ID NO: 50) | |
| 6 | RD | LEVVAA(X)ᵤLLISW(X)ᵥYRITY(X)_{w}FTV | SEQ ID NO: 30 |
| | | (X)ₓATISGL(X)_{y}YTITVYA(X)_{z}ISINYRT | |
| | | (SEQ ID NO: 50) | |
| 7 | D | LEVVAA(X)ᵤLLISW(X)ᵥYRITY(X)_{w}FTV | SEQ ID NO: 31 |
| | | (X)ₓATISGL(X)_{y}YTITVYA(X)_{z}ISINYRT | |
| | | (SEQ ID NO: 50) | |
| 8 | — | LEVVAA(X)ᵤLLISW(X)ᵥYRITY(X)_{w}FTV | SEQ ID NO: 32 |
| | | (X)ₓATISGL(X)_{y}YTITVYA(X)_{z}ISINYRT | |
| | | (SEQ ID NO: 50) | |

Table 4 shows the amino acid sequences of the degenerate human ¹⁰Fn3 molecules of Table 3 including a C-terminal region consisting of SEQ ID NO: 55 or 56.

TABLE 4
Amino acid sequences of degenerate wild-type (or mutated)
human ¹⁰Fn3 molecules with various N-terminal
sequences and either of two C-terminal tail sequences

| | N-terminal | Degenerate wild-type human | C-terminal | |
|---|---|---|---|---|
| Version | region | ¹⁰Fn3 Core Domain | region | Full length |
| 1 | VSDVPRD | LEVVAA(X)ᵤLLISW(X)ᵥYRI | EIDKPSQ | SEQ ID NO: |
| | (SEQ ID | TY(X)_{w}FTV(X)ₓATISGL(X)_{y}Y | (SEQ ID NO: | 33 |
| | NO: 51) | TITVYA(X)_{z}ISINYRT (SEQ | 55) | |
| | | ID NO: 50) | EIEKPSQ | SEQ ID NO: |
| | | | (SEQ ID NO: | 34 |
| | | | 56) | |
| 2 | SDVPRD | LEVVAA(X)ᵤLLISW(X)ᵥYRI | EIDKPSQ | SEQ ID NO: |
| | (SEQ ID | TY(X)_{w}FTV(X)ₓATISGL(X)_{y}Y | (SEQ ID NO: | 35 |
| | NO: 52) | TITVYA(X)_{z}ISINYRT (SEQ | 55) | |
| | | ID NO: 50) | EIEKPSQ | SEQ ID NO: |
| | | | (SEQ ID NO: | 36 |
| | | | 56) | |
| 3 | DVPRD | LEVVAA(X)ᵤLLISW(X)ᵥYRI | EIDKPSQ | SEQ ID NO: |
| | (SEQ ID | TY(X)_{w}FTV(X)ₓATISGL(X)_{y}Y | (SEQ ID NO: | 37 |
| | NO: 53) | TITVYA(X)_{z}ISINYRT (SEQ | 55) | |
| | | ID NO: 50) | EIEKPSQ | SEQ ID NO: |
| | | | (SEQ ID NO: | 38 |
| | | | 56) | |
| 4 | VPRD | LEVVAA(X)ᵤLLISW(X)ᵥYRI | EIDKPSQ | SEQ ID NO: |
| | (SEQ ID | TY(X)_{w}FTV(X)ₓATISGL(X)_{y}Y | (SEQ ID NO: | 39 |
| | NO: 54) | TITVYA(X)_{z}ISINYRT (SEQ | 55) | |
| | | ID NO: 50) | EIEKPSQ | SEQ ID NO: |
| | | | (SEQ ID NO: | 40 |
| | | | 56) | |
| 5 | PRD | LEVVAA(X)ᵤLLISW(X)ᵥYRI | EIDKPSQ | SEQ ID NO: |
| | | TY(X)_{w}FTV(X)ₓATISGL(X)_{y}Y | (SEQ ID NO: | 41 |
| | | TITVYA(X)_{z}ISINYRT (SEQ | 55) | |
| | | ID NO: 50) | EIEKPSQ | SEQ ID NO: |
| | | | (SEQ ID NO: | 42 |
| | | | 56) | |
| 6 | RD | LEVVAA(X)ᵤLLISW(X)ᵥYRI | EIDKPSQ | SEQ ID NO: |
| | | TY(X)_{w}FTV(X)ₓATISGL(X)_{y}Y | (SEQ ID NO: | 43 |
| | | TITVYA(X)_{z}ISINYRT (SEQ | 55) | |
| | | ID NO: 50) | EIEKPSQ | SEQ ID NO: |
| | | | (SEQ ID NO: | 44 |
| | | | 56) | |
| 7 | D | LEVVAA(X)ᵤLLISW(X)ᵥYRI | EIDKPSQ | SEQ ID NO: |
| | | TY(X)_{w}FTV(X)ₓATISGL(X)_{y}Y | (SEQ ID NO: | 45 |
| | | TITVYA(X)_{z}ISINYRT (SEQ | 55) | |
| | | ID NO: 50) | EIEKPSQ | SEQ ID NO: |
| | | | (SEQ ID NO: | 46 |
| | | | 56) | |
| 8 | — | LEVVAA(X)ᵤLLISW(X)ᵥYRI | EIDKPSQ | SEQ ID NO: |
| | | TY(X)_{w}FTV(X)ₓATISGL(X)_{y}Y | (SEQ ID NO: | 47 |
| | | TITVYA(X)_{z}ISINYRT (SEQ | 55) | |
| | | ID NO: 50) | EIEKPSQ | SEQ ID NO: |
| | | | (SEQ ID NO: | 48 |
| | | | 56) | |

In SEQ ID NOs: 25-48 and 50, the AB loop is represented by (X)ᵤ, the BC loop is represented by (X)ᵥ, the CD loop is represented by (X)_{w}, the DE loop is represented by (X)ₓ, the EF loop is represented by (X)_{y} and the FG loop is represented by X_{z}. X represents any amino acid and the subscript following the X represents an integer of the number of amino acids. In particular, u, v, w, x, y and z may each independently be anywhere from 2-20, 2-15, 2-10, 2-8, 5-20, 5-15, 5-10, 5-8, 6-20, 6-15, 6-10, 6-8, 2-7, 5-7, or 6-7 amino acids. The sequences of the beta strands (underlined) may have anywhere from 0 to 10, from 0 to 8, from 0 to 6, from 0 to 5, from 0 to 4, from 0 to 3, from 0 to 2, or from 0 to 1 substitutions, deletions or additions across all 7 scaffold regions relative to the corresponding amino acids shown in SEQ ID NOs: 25-48 or 50. In some embodiments, the sequences of the beta strands may have anywhere from 0 to 10, from 0 to 8, from 0 to 6, from 0 to 5, from 0 to 4, from 0 to 3, from 0 to 2, or from 0 to 1 substitutions, e.g., conservative substitutions, across all 7 scaffold regions relative to the corresponding amino acids shown in SEQ ID NO: 25-48 or 50.

In certain embodiments, the hydrophobic core amino acid residues (bolded residues in SEQ ID NO: 25 above) are fixed, and any substitutions, conservative substitutions, deletions or additions occur at residues other than the hydrophobic core amino acid residues. Thus, in some embodiments, the hydrophobic core residues of the polypeptides provided herein have not been modified relative to the wild-type human ¹⁰Fn3 domain (e.g., SEQ ID NO: 1).

In some embodiments, an FBS moiety comprises a ¹⁰Fn3 domain, wherein the ¹⁰Fn3 domain comprises a loop, AB; a loop, BC; a loop, CD; a loop, DE; a loop, EF; and a loop, FG; and has at least one loop selected from loop AB, BC, CD, DE, EF and FG with an altered amino acid sequence relative to the sequence of the corresponding loop of the wild-type human ¹⁰Fn3 domain. In some embodiments, a single loop is altered. In some embodiments, at most 2 loops are altered. In some embodiments, at most 3 loops are altered. In some embodiments, the BC, DE and/or FG loops are altered. In certain embodiments, the AB, CD and EF loops are altered. In certain embodiments, the FG loop is the only loop that is altered. In other embodiments, the CD and FG loops are both altered, and optionally, no other loops are altered. In certain embodiments, the CD and EF loops are both altered, and optionally, no other loops are altered. In some embodiments, one or more specific scaffold alterations are combined with one or more loop alterations. By “altered” is meant one or more amino acid sequence alterations relative to a template sequence (i.e., the corresponding wild-type human fibronectin domain) and includes amino acid additions, deletions, and substitutions. Exemplary ¹⁰Fn3 molecules comprising specific combinations of altered loops and/or scaffold regions (e.g., beta strands, N-terminal region and C-terminal region) are further disclosed herein.

It should be understood that not every residue within a loop region needs to be modified in order to achieve a ¹⁰Fn3 binding domain having strong affinity for a desired target. Additionally, insertions and deletions in the loop regions may also be made while still producing high affinity ¹⁰Fn3 binding domains.

In some embodiments, one or more loops selected from AB, BC, CD, DE, EF and FG may be extended or shortened in length relative to the corresponding loop in wild-type human ¹⁰Fn3. In any given polypeptide, one or more loops may be extended in length, one or more loops may be reduced in length, or combinations thereof. In some embodiments, the length of a given loop may be extended by 2-25, 2-20, 2-15, 2-10, 2-5, 5-25, 5-20, 5-15, 5-10, 10-25, 10-20, or 10-15 amino acids. In some embodiments, the length of a given loop may be reduced by 1-15, 1-11, 1-10, 1-5, 1-3, 1-2, 2-10, or 2-5 amino acids. In particular, the FG loop of ¹⁰Fn3 is 13 residues long, whereas the corresponding loop in antibody heavy chains ranges from 4-28 residues. To optimize antigen binding in polypeptides relying on the FG for target binding, therefore, the length of the FG loop of ¹⁰Fn3 may be altered in length as well as in sequence to obtain the greatest possible flexibility and affinity in target binding.

In some embodiments, the FBS moiety comprises a ¹⁰Fn3 domain wherein the non loop regions comprise an amino acid sequence that is at least 80, 85, 90, 95, 98, or 100% identical to the non-loop regions of SEQ ID NO: 1 or 9, wherein at least one loop selected from AB, BC, CD, DE, EF and FG is altered. For example, in certain embodiments, the AB loop may have up to 4 amino acid substitutions, up to 10 amino acid insertions, up to 3 amino acid deletions, or a combination thereof; the BC loop may have up to 10 amino acid substitutions, up to 4 amino acid deletions, up to 10 amino acid insertions, or a combination thereof; the CD loop may have up to 6 amino acid substitutions, up to 10 amino acid insertions, up to 4 amino acid deletions, or a combination thereof; the DE loop may have up to 6 amino acid substitutions, up to 4 amino acid deletions, up to 13 amino acid insertions, or a combination thereof; the EF loop may have up to 5 amino acid substitutions, up to 10 amino acid insertions, up to 3 amino acid deletions, or a combination thereof; and/or the FG loop may have up to 12 amino acid substitutions, up to 11 amino acid deletions, up to 25 amino acid insertions, or a combination thereof.

In some embodiments, an FBS moiety comprises a ¹⁰Fn3 domain having at least 40%, 50%, 55%, 60%, 65%, 70%, 75%, 80%, 85%, or 90% identity to a human ¹⁰Fn3 domain having an amino acid sequence selected from the group of sequence comprising SEQ ID NOs: 1-50. In certain embodiments, the FBS moiety provided herein has at least 50% identity to an amino acid sequence selected from the group of amino acid sequences comprising SEQ ID NO: 1-50. In other embodiments, the FBS moiety has at least 65% identity to an amino acid sequence selected from the group of amino acid sequences comprising SEQ ID NO: 1-50. In certain embodiments, one or more of the loops will not be modified relative to the sequence of the corresponding loop of the wild-type sequence and/or one or more of the β-strands will not be modified relative to the sequence of the corresponding β-strand of the wild-type sequence and/or the N-terminal or C-terminal regions will not be modified. In certain embodiments, each of the beta or beta-like strands of a ¹⁰Fn3 domain in an FBS moiety may comprise, consist essentially of, or consist of an amino acid sequence that is at least 80%, 85%, 90%, 95% or 100% identical to the sequence of a corresponding beta or beta-like strand of SEQ ID NO: 1. Preferably, variations in the β-strand regions will not disrupt the stability of the polypeptide in physiological conditions.

In some embodiments, the non-loop region of a ¹⁰Fn3 domain may be modified by one or more conservative substitutions. As many as 5%, 10%, 20% or even 30% or more of the amino acids in the ¹⁰Fn3, domain may be altered by a conservative substitution without substantially altering the affinity of the ¹⁰Fn3 for a ligand. In certain embodiments, the non-loop regions, e.g., the β-strands may comprise anywhere from 0-15, 0-10, 0-8, 0-6, 0-5, 0-4, 0-3, 1-15, 1-10, 1-8, 1-6, 1-5, 1-4, 1-3, 2-15, 2-10, 2-8, 2-6, 2-5, 2-4, 5-15, or 5-10 conservative amino acid substitutions. In exemplary embodiments, the scaffold modification may reduce the binding affinity of the ¹⁰Fn3 binder for a ligand by less than 100-fold, 50-fold, 25-fold, 10-fold, 5-fold, or 2-fold. It may be that such changes may alter the immunogenicity of the ¹⁰Fn3 in vivo, and where the immunogenicity is decreased, such changes may be desirable. As used herein, “conservative substitutions” are residues that are physically or functionally similar to the corresponding reference residues. That is, a conservative substitution and its reference residue have similar size, shape, electric charge, chemical properties including the ability to form covalent or hydrogen bonds, or the like. Exemplary conservative substitutions include those fulfilling the criteria defined for an accepted point mutation in Dayhoff et al., Atlas of Protein Sequence and Structure, 5:345-352 (1978 and Supp.). Examples of conservative substitutions include substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

Also provided herein are ¹⁰Fn3 domains having combinations of loop and scaffold modifications. Conjugates may comprise a ¹⁰Fn3, domain comprising (i) a modification in the amino acid sequence of at least one of loops AB, BC, CD, DE, EF, or FG, and (ii) a modification in the amino acid sequence of at least one scaffold region (i.e., a modification in at least one β-strand, the N-terminal region, and/or the C-terminal region), wherein the modified loop(s) and modified scaffold region(s) both contribute to binding the same target. In exemplary embodiments, the scaffold region modifications are located adjacent to modifications in a loop region, e.g., if the AB loop is modified, scaffold mutations may tend to be located in β-strand A and/or β-strand B, which are adjacent to the AB loop in the linear sequence of the ¹⁰Fn3 domain. In other embodiments, a cluster of modifications may be found together in loop and scaffold regions that are adjacent to one another in the linear sequence of the Fn3 domain. For example, Fn3 binders having both loop and scaffold modifications, may have clusters of amino acid modifications in the following combinations of loop and scaffold regions that are adjacent to each other in the linear sequence of the Fn3 domain: β-strand/loop/β-strand, loop/β-strand/loop, loop/β-strand/loop/β-strand, terminal region/β-strand/loop, or loop/β-strand/terminal region, etc. For example, Fn3 domains having novel combinations of loop and scaffold modifications may have clusters of modifications such that over a stretch of 20 contiguous amino acids at least 15 of the amino acids are modified relative to wild-type. In other embodiments, at least 17 out of 20, 18 out of 20, 17 out of 25, 20 out of 25, or 25 out of 30 residues in a contiguous stretch are modified relative to the wild-type Fn3 domain sequence over the corresponding stretch of amino acids. In certain embodiments, a given Fn3 domain may have two or three clusters of modifications separated by stretches of unmodified (i.e., wild-type) sequence. For any given region (i.e., a loop, β-strand or terminal region) that is modified, all or only a portion of the region may be modified relative to the wild-type sequence. When a 3-strand region is modified, preferably the hydrophobic core residues remain unmodified (i.e., wild-type) and one or more of the non-core residues in the β-strand are modified.

In some embodiments, ¹⁰Fn3 domains comprise a binding face along the “west-side” of the molecule (“West-side binders” or “WS binders”). WS binders may comprise a modified CD loop and a modified FG loop, as compared to the corresponding CD and FG loop sequences set forth in SEQ ID NO: 1 or 9. The CD loop and the FG loop both contribute to binding to the same target. In certain embodiments, the WS binders may comprise additional modifications at one or more regions within the Fn3 domain. For example, WS binders may comprise scaffold modifications in one or more of the β-strand regions adjacent to the CD and/or FG loops. In particular, WS binders may comprise sequence modifications in one or more of β-strand C, β-strand D, β-strand F, and/or β-strand G. Exemplary scaffold modifications include modifications at one or more scaffold region positions corresponding to the amino acid positions: 33, 35, 49, 69, 71, 73, 89 and/or 91 of SEQ ID NO: 1 or 9. The WS binders may also comprise modifications in the BC loop, particularly in the C-terminal portion of the BC loop. In one embodiment, the last two residues of the BC loop (i.e., corresponding to amino acids 30 and 31 in the wild-type ¹⁰Fn3 domain) are modified relative to the wild-type sequence. All or a portion of the additional loop and scaffold modifications may contribute to binding to the target in conjunction with the modified CD and FG loops. Preferably, the hydrophobic core residues are not modified relative to the wild-type sequence.

Exemplary WS binders include those having a wild-type or mutated amino acid at positions 30, 31, 33, 35, 37, 38, 46, 47, 49, 50, 67, 69, 71, 73, 75, 76, 84, 85, 86, 87, 89 or 91.

In some embodiments, a ¹⁰Fn3 domain comprises modifications in the CD, DE and, in some cases, EF loops, wherein the loop modifications all contribute to target binding. These polypeptides are referred to as “front binders”. The front binders may additionally comprise modifications in one or more scaffold regions, particularly in scaffold regions that flank or are adjacent to a modified loop region. For example, the front binders may comprise a scaffold modification in one or more of β-strand C, β-strand D, and/or β-strand E relative to the sequences of the corresponding β-strands of the wild-type Fn3 domain, e.g., human ¹⁰Fn3 domain (SEQ ID NO: 1 or 9). Preferably the hydrophobic core residues are not modified relative to the wild-type sequence. Exemplary scaffold modifications that may be present in front binders, include modifications at one or more positions corresponding to amino acid positions 36, 49, 58 and/or 50 of SEQ ID NO: 1 or 9. Such scaffold modifications may contribute to binding to the target together with the modified loops. In certain embodiments, the front binders may comprise clusters of modifications spanning several loop and strand regions of the Fn3, e.g., ¹⁰Fn3 domain. In particular, the front binders may comprise modifications in at least 15, 20, 24, 25, or 27 of the 31 residues between the amino acids corresponding to residues 36 through 66 of the wild-type Fn3, e.g., human ¹⁰Fn3, domain (SEQ ID NO: 1 or 9). The loop and/or strand modifications may include amino acid substitutions, deletions and/or insertions, or combinations thereof. In exemplary embodiments, the CD loop is extended in length or reduced in length relative to the CD loop of the Fn3, e.g., wild-type human ¹⁰Fn3, domain (SEQ ID NO: 1 or 9).

In some embodiments, ¹⁰Fn3 domains comprise modifications in the EF and FG loops, wherein the loop modifications contribute to binding the same target. These polypeptides are referred to as “back binders” herein. The back binders may comprise additional modifications in other loop and/or scaffold regions. For example, a back binder may contain modifications in at least a portion of the AB loop, preferably the N-terminal portion of the AB loop. In an exemplary embodiment, the first two amino acids of the AB loop (i.e., corresponding to amino acid residues 14 and 15 of the wild-type ¹⁰Fn3 domain) are modified relative to the wild-type sequence. In certain embodiments, a back binder may also contain one or more scaffold modifications, particularly modifications in one or more scaffold regions that are adjacent to a modified loop region. For example, back binders may contain one or more modifications in one or more of 3-strand A, β-strand G, the N-terminal region, and/or the C-terminal region. Preferably the hydrophobic core residues are not modified relative to the wild-type sequence. Exemplary scaffold modifications include modifications at one or more positions corresponding to amino acid positions 1-7, 9-13, 89, 91, 93 and/or 94 of SEQ ID NO: 1 or 9. One or more of the additional loop and/or scaffold modifications may contribute to binding to the target along with the modified EF and FG loops. Suitable loop and/or scaffold region modifications include amino acid substitutions, deletions and/or insertions, or combinations thereof. In certain embodiments, the amino acid sequence of the FG loop is extended in length or reduced in length relative to the FG loop of the wild-type human ¹⁰Fn3 domain (SEQ ID NO: 1 or 9).

In certain embodiments, a back binder may comprise a cluster of modified amino acid residues over a contiguous span of several regions in the ¹⁰Fn3 domain. For example, at least 14 of the first 15 amino acid residues of the Fn3, e.g., ¹⁰Fn3, domain may be modified relative to the corresponding residues in the wild-type Fn3, e.g., human ¹⁰Fn3, domain (SEQ ID NO: 1 or 9), and/or at least 15 of the 18 residues between the amino acids corresponding to residues 80 through 97 (or 94) of the wild-type Fn3, e.g., human ¹⁰Fn3, domain (SEQ ID NO: 1 or 9) may be modified relative to the corresponding residues in the wild-type sequence.

In certain embodiments, a ¹⁰Fn3 domain comprises modifications in the amino acid sequences of β-strand A, loop AB, β-strand B, loop CD, β-strand E, loop EF, and β-strand F, relative to the sequences of the corresponding regions of the wild-type sequence. These polypeptides are referred to as “south pole binders” or “SP binders” herein. The modified loops and strands contribute to binding to the same target. The amino acid sequence of the CD loop may be extended in length or reduced in length relative to the CD loop of the wild-type Fn3, e.g., human ¹⁰Fn3, domain (SEQ ID NO: 1 or 9). The south pole binders may comprise additional modifications in β-strand G and/or the C-terminal region relative to the sequence of the corresponding region of the wild-type sequence. In exemplary embodiments, the south pole binders may comprise one or more modifications at amino acids corresponding to positions 11, 12, 19, 60, 61, 69, 91, 93 and 95-97 of the wild-type sequence.

In some embodiments, a ¹⁰Fn3 domain comprises modified BC, DE and FG loops, as compared to the corresponding BC, DE and FG loop sequences set forth in SEQ ID NO: 1 or 9, as well as additional modifications in one or more of β-strand C, β-strand D, β-strand F and β-strand G strand residues. The β-strand and loop region modifications together contribute to binding to the target. These proteins are referred to as “Northwest binders”, or “NW binders”, herein. In exemplary embodiments, the NW binders comprise one or more scaffold modifications at any one of, or combination of, amino acid positions corresponding to scaffold region positions R33, T49, Y73 and S89 of SEQ ID NO: 1 or 9. Suitable modifications in loop and scaffold regions include amino acid substitutions, deletions and/or insertions, or combinations thereof. In certain embodiments, one or more of the BC, DE and FG loops are extended in length or reduced in length, or combinations thereof, relative to the wild-type sequence. In one embodiment, each of the BC, DE and FG loops are extended in length or reduced in length, or combinations thereof, relative to the wild-type sequence (e.g., SEQ ID NO: 1 or 9). In certain embodiments, only a portion of the BC loop is modified, particularly the C-terminal portion, relative to the wild-type sequence. For example, the BC loop may be modified only at amino acid residues corresponding to amino acids 27-31 of the wild-type BC loop, whereas the rest of the BC loop (i.e., corresponding to residues 23-26 of the wild-type loop) are left unmodified.

In some embodiments, a ¹⁰Fn3 domain comprises a modified BC, DE and FG loop as well as one or more additional modifications in any one of, or combination of, the N-terminal region, β-strand A, β-strand B and/or β-strand E. These proteins are referred to as “Northeast binders”, or “NE binders”, herein. In exemplary embodiments, the NE binders are modified at any one of, or combination of, amino acids corresponding to scaffold region positions 1-7, E9, L19, S21 and/or T58 of the wild-type sequence (SEQ ID NO: 1 or 9). The combination of modified loop and scaffold regions contributes to binding to the target.

In some embodiments, a ¹⁰Fn3 domain comprises modifications in one or more of the AB, CD, DE and EF loops, as well as additional modifications in one or more of 3-strand B, β-strand D and/or β-strand E. These proteins are referred to as “South Front binders” herein. The combination of modified loop and strand residues contributes to binding to the target. In exemplary embodiments, a South Front binder may be modified at one or more amino acid positions corresponding to scaffold region positions L19, T49, T58, S60, and/or G61 of SEQ ID NO: 1 or 9 and/or at one or more amino acid positions corresponding to loop region positions T14-S17, P51, T56, G40-E47, and/or K63-G65 of SEQ ID NO: 1 or 9. In exemplary embodiments, a South Front binder may be extended in length or reduced in length in the AB loop, between amino acids corresponding to residues 18 and 20 of the wild-type sequence, and/or in the CD loop.

In some embodiments, a ¹⁰Fn3 domain comprises a modified β-strand A and β-strand G, as compared to the corresponding strand of SEQ ID NO: 1 or 9. These proteins are referred to as “AG Binders” or “AG Strand” binders herein. In certain embodiments, the AG strand binders comprise clusters of modifications at the N-terminal and C-terminal portions of the Fn3, e.g., ¹⁰Fn3, 0.5 domain, whereas the middle portion of the Fn3 remains unmodified. For example, an AG strand binder may comprise modifications at 16 out of 19 of the first 19 amino acids in the ¹⁰Fn3 domain (i.e., corresponding to amino acid positions 1-19 of SEQ ID NO: 1 or 9) and modifications at 13-17 out of 18 of the last 18 amino acids in the ¹⁰Fn3 domain (i.e., corresponding to amino acid positions 84-101 of SEQ ID NO: 9) or at 14-18 out of 22 of the last 22 amino acids in the ¹⁰Fn3 domain (i.e., corresponding to amino acid positions 80-101 of SEQ ID NO: 9). In exemplary embodiments, an AG binder may comprise modifications at one or more positions corresponding to positions 1-7, 9, 11-17, 19, 84-89 and 91-97 of SEQ ID NO: 9. Preferably the modified regions in an AG binder contribute to binding to the same target.

In some embodiments, a ¹⁰Fn3 domain comprises a modified CD and EF loop, as well as additional modifications in any one of, or combination of residues corresponding to positions 69 or 91-97 of SEQ ID NO: 1 or 9. These proteins are referred to as “Southwest binders”, or “SW binders”, herein. The modified loop and scaffold regions contribute to binding to the target.

In certain embodiments, fusion proteins comprise a ¹⁰Fn3 domain having reduced immunogenicity, wherein a portion of the BC loop is left as wild-type. Preferably such polypeptides have lower immunogenicity relative to an equivalent polypeptide with modifications in a greater portion of the BC loop. In exemplary embodiments, the N-terminal portion of the BC loop is left as wild-type. For example, the first 1, 2, 3, 4, 5, or 5 residues of the BC loop may be left as wild-type, while the remaining C-terminal residues of the BC loop can be modified. In Fn3 designs having at least a portion of the N-terminal region of the BC loop as wild-type, it may be desirable to leave all or a portion of β-strand B and/or β-strand C unmodified relative to the wild-type sequence as well, particularly the portions of β-strand B and/or β-strand C that are adjacent to the BC loop (i.e., the C-terminal portion of β-strand B and/or the N-terminal portion of β-strand C). In exemplary embodiments, Fn3 domains having the wild-type sequence in an N-terminal portion of the BC loop and reduced immunogenicity may not have any modifications in the N-terminal region, β-strand A, AB loop, and β-strand B. In Fn3 designs with a portion of the BC loop as wild-type, the modified portion of the BC loop may contribute to target binding along with modifications in other regions of the ¹⁰Fn3 domain.

In certain embodiments, fusion proteins comprise a ¹⁰Fn3 domain having reduced immunogenicity, wherein the strong HLA anchor in the region of β-strand B/BC loop/β-strand C (the “BC anchor”) has been removed or destroyed (e.g., modified relative to the wild-type sequence in a manner that reduces binding affinity to one or more HLA receptors). For example, the BC anchor may be removed or destroyed by modifying the Fn3, e.g., ¹⁰Fn3, 0.5 domain at one or more positions corresponding to positions L19, S21, R33 and/or T35 of SEQ ID NO:1 or 9. When the BC anchor has been removed or destroyed, it is possible to modify the sequence of the BC loop without significantly increasing the immunogenic potential of the BC region. Accordingly, many such Fn3 designs have modifications in the BC loop in addition to the modifications in β-strand B and/or β-strand C. The BC loop may contribute to target binding, optionally in combination with modifications in other regions of the Fn3 domain. The modifications in β-strand B and/or β-strand C may or may not contribute to target binding.

In exemplary embodiments, a ¹⁰Fn3 domain binds to a desired target with a K_{d} of less than 500 nM, 100 nM, 50 nM, 10 nM, 5 nM, 1 nM, 500 pM, 100 pM or less. In some embodiments, the ¹⁰Fn3 domain binds to a desired target with a K_{d} between 1 pM and 1 μM, between 100 pM and 500 nM, between 1 nM and 500 nM, or between 1 nM and 100 nM. In exemplary embodiments, the FBS moiety binds specifically to a target that is not bound by a wild-type ¹⁰Fn3 domain, particularly the wild-type human ¹⁰Fn3 domain having, e.g., SEQ ID NO: 1-8, 9, 11, 13, 15, 17, 19, 21, or 23.

In certain embodiments, an FBS moiety comprises an amino acid sequence that is at least 40%, 50%, 60%, 70%, 75%, 80%, 85%, 90%, 95%, 97%, 98%, or 99% identical to an amino acid sequence selected from the group of sequences consisting of SEQ ID NOs: 1-50, and the FBS binds specifically to a target, e.g., with a K_{d} of less than 500 nM, 100 nM, 50 nM, 10 nM, 5 nM, 1 nM, 500 pM, 100 pM or less. The FBS moiety may comprise amino acid changes (or alterations) in one or more loops and one or more scaffold regions.

In some embodiments, one or more residues of the integrin-binding motif “arginine-glycine-aspartic acid” (RGD) (amino acids 78-80 of SEQ ID NO: 1) may be substituted so as to disrupt integrin binding. In some embodiments, the FG loop of the polypeptides provided herein does not contain an RGD integrin binding site. In one embodiment, the RGD sequence is replaced by a polar amino acid-neutral amino acid-acidic amino acid sequence (in the N-terminal to C-terminal direction). In another embodiment, the RGD sequence is replaced with SGE or RGE.

In some embodiments, the amino acid sequences of the N-terminal and/or C-terminal regions of an FBS moiety are modified by deletion, substitution or insertion relative to the amino acid sequences of the corresponding regions of ¹⁰Fn3 domains comprising, e.g., SEQ ID NO: 1 or 9.

In certain embodiments, the amino acid sequence of the first 1, 2, 3, 4, 5, 6, 7, 8 or 9 residues of SEQ ID NO: 1 may be modified or deleted in the polypeptides provided herein relative to the sequence of the corresponding amino acids in the wild-type human ¹⁰Fn3 domain having SEQ ID NO: 1 or 9. In exemplary embodiments, the amino acids corresponding to amino acids 1-8 or 9 of any one of SEQ ID NOs: 1-50 are replaced with an alternative N-terminal region having from 1-20, 1-15, 1-10, 1-8, 1-5, 1-4, 1-3, 1-2, or 1 amino acids in length. Exemplary alternative N-terminal regions include (represented by the single letter amino acid code) M, MG, G, MGVSDVPRDL (SEQ ID NO: 57) and GVSDVPRDL (SEQ ID NO: 58), or N-terminal truncations of any one of SEQ ID NOs: 57 and 58. Other suitable alternative N-terminal regions include, for example, XₙSDVPRDL (SEQ ID NO: 59), XₙDVPRDL (SEQ ID NO: 60), XₙVPRDL (SEQ ID NO: 61), XₙPRDL (SEQ ID NO: 62), XₙRDL (SEQ ID NO: 63), XₙDL (SEQ ID NO: 64), or XₙL, wherein n=0, 1 or 2 amino acids, wherein when n=1, X is Met or Gly, and when n=2, X is Met-Gly. When a Met-Gly sequence is added to the N-terminus of a ¹⁰Fn3 domain, the M will usually be cleaved off, leaving a G at the N-terminus. In other embodiments, the alternative N-terminal region comprises the amino acid sequence MASTSG (SEQ ID NO: 65).

As further described herein, in some embodiments, the first eight residues (i.e., residues 1-8) of SEQ ID NO: 1 are deleted, generating a ¹⁰Fn3 domain having the amino acid sequence of SEQ ID NO: 8. Additional sequences may also be added to the N- or C-terminus of a ¹⁰Fn3 domain having the amino acid sequence of any one of SEQ ID NOs: 1-50. For example, in some embodiments, the N-terminal extension consists of an amino acid sequence selected from the group consisting of: M, MG, and G. For example, any one of SEQ ID NO: 1-50 may be preceded by M, MG, or G.

As further discussed herein, in certain embodiments, an FBS moiety comprises SEQ ID NO: 1 and one or more amino acid residues at the —C-terminus, e.g., the amino acid residues 1, 1-2, 1-3, 1-4, 1-5, 1-6 or 1-7 of EIDKPSQ (SEQ ID NO: 55) or EIEKPSQ (SEQ ID NO: 56), or of SEQ ID NO: 55 or 56 in which at most or exactly 1, 2, 3, 4, 5, or 6 amino acids are substituted, added or deleted. In exemplary embodiments, a C-terminal region having from 1-20, 1-15, 1-10, 1-8, 1-5, 1-4, 1-3, 1-2, or 1 amino acids in length is added to any one of SEQ ID NOs: 1-50. In addition to SEQ ID NOs: 55 and 56, specific examples of C-terminal region sequences include, for example, polypeptides comprising, consisting essentially of, or consisting of, EIEK (SEQ ID NO: 66), EGSGC (SEQ ID NO: 67), EIEKPCQ (SEQ ID NO: 68), EIEKP (SEQ ID NO: 69), EIEKPS (SEQ ID NO: 70), EIEKPC (SEQ ID NO: 72), or HHHHHH (SEQ ID NO: 72). In some embodiments, the alternative C-terminal region comprises EIDK (SEQ ID NO: 73), and in particular embodiments, the alternative C-terminal region is EIDKPCQ (SEQ ID NO: 74).

In certain embodiments, an FBS moiety comprises a ¹⁰Fn3 core domain having both an alternative N-terminal region sequence and an alternative C-terminal region sequence.

In certain embodiments, an FBS moiety is based on an Fn3 repeat other than the 10^{th} repeat of the type III domain of fibronectin, e.g., human fibronectin. For example, an FBS moiety may be similar to any of the other fibronectin type III repeats, e.g., the 1^{st}, 2^{nd}, 3^{rd}, 4^{th}, 5^{th}, 6^{th}, 7^{th}, 8^{th}, 9^{th}, 11^{th}, 12^{th}, 13^{th}, 14^{th}, 15^{th}, 16^{th}, 17^{th}, and 18^{th} Fn3 repeats. In yet other embodiments, an FBS moiety may be from a molecule other than fibronectin. Exemplary FBS moieties may be derived from tenascin, a protein that is composed of 15 Fn3 domains with similar sequence similarities to one another as found in fibronectin. These repeats are described, e.g., in Jacobs et al., Protein Engineering, Design & Selection, 25:107 (2012). Based on the homology of the repeats in the fibronectin molecule and those in the tenascin molecule, artificial molecules based on these homologies have been created. Proteins comprising a consensus amino acid sequence based on the homology of the domains in the fibronectin molecule are referred to as Fibcon and FibconB (WO 2010/093627 and Jacobs et al. (2012) supra.) and those based on the homology of the domains in the tenascin molecule are referred to as Tencon. An exemplary Fibcon amino acid sequence comprises the following amino acid sequence:

MPAPTDLRFTNETPSSLLISWTPPRVQITGYIIRYGPVGSDGRVKEFTVP
PSVSSATITGLKPGTEYTISVIALKDNQESEPLRGRVTTGG
(FibconB; SEQ ID NO: 75),

wherein loop AB consists of amino acids 13-16 (TPSS; SEQ ID NO: 76), loop BC consists of amino acids 22-28 (TPPRVQI; SEQ ID NO: 77), loop CD consists of amino acids 38-43 (VGSDGR; SEQ ID NO: 78), loop DE consists of amino acids 51-54 (PSVS; SEQ ID NO: 79), loop EF consists of amino acids 60-64 (GLKPG; SEQ ID NO: 80) and loop FG consist of amino acids 75-81 (KDNQESEP; SEQ ID NO: 81). Another Fibcon amino acid sequence comprises the following amino acid sequence:

LDAPTDLQVTNVTDTSITVSWTPPSATITGYRITYTPSNGPGEPKELTVP
PSSTSVTITGITPGVEYVVSVYALKDNQESPPLVGTCTT (SEQ ID
NO: 82; Jacobs et al., supra).

Tenascin derived Fn3 proteins include Tencons (WO 2010/051274, WO 2010/051310 and WO 2011/137319, which are specifically incorporated by reference herein). An exemplary Tencon protein has the following amino acid sequence:

LPAPKNLVVSEVTEDSLRLSWTAPDAAFDSFLIQYQESEKVGEAINLTVP
GSERSYDLTGLKPGTEYTVSIYGVKGGHRSNPLSAEFTT (SEQ ID
NO: 83; Jacobs et al.,supra, and WO 2011/137319),

wherein loop AB consists of amino acids 13-16 (TEDS; SEQ ID NO: 84, loop BC consists of amino acids 22-28 (TAPDAAF; SEQ ID NO: 85), loop CD consists of amino acids 38-43 (SEKVGE; SEQ ID NO: 86), loop DE consists of amino acids 51-54 (GSER; SEQ ID NO: 87), loop EF consists of amino acids 60-64 (GLKPG; SEQ ID NO: 88) and loop FG consists of amino acids 75-81 (KGGHRSN; SEQ ID NO: 89).

A Fibcon, FibconB or Tencon moiety, or target binding variants thereof, whether by themselves or linked to a heterologous moiety may be fused as described herein. Fn3 domains from other proteins, e.g., cell surface hormone and cytokine receptors, chaperonins, and carbohydrate-binding domains, may be conjugated as described herein.

FBS proteins or moieties are described, e.g., in WO 2010/093627, WO 2011/130324, WO 2009/083804, WO 2009/133208, WO 02/04523, WO 2012/016245, WO 2009/023184, WO 2010/051310, WO 2011/020033, WO 2011/051333, WO 2011/051466, WO 2011/092233, WO 2011/100700, WO 2011/130324, WO 2011/130328, WO 2011/137319, WO 2010/051274, WO 2009/086116 and WO 09/058379. (all of which are specifically incorporated by reference herein): any of the FBS proteins or moieties described in these publications may be refolded as described herein.

In certain embodiments, a fusion proteins comprises at least 2 FBS moieties, e.g., the fusion protein comprises a multivalent FBS moiety. For example, a multivalent FBS may comprise 2, 3 or more FBS moieties, e.g., ¹⁰Fn3 domains, that are covalently associated. In exemplary embodiments, the FBS moiety is a bispecific or dimeric protein comprising two ¹⁰Fn3 domains.

The FBS moieties, e.g., ¹⁰Fn3 domains, in a multivalent protein may be connected by a polypeptide linker. Exemplary polypeptide linkers include polypeptides having from 1-20, 1-15, 1-10, 1-8, 1-5, 1-4, 1-3, or 1-2 amino acids. Suitable linkers for joining the ¹⁰Fn3 domains are those which allow the separate domains to fold independently of each other forming a three dimensional structure that permits high affinity binding to a target molecule. Specific examples of suitable linkers include glycine-serine based linkers, glycine-proline based linkers, proline-alanine based linkers as well as any other linkers described herein. In some embodiments, the linker is a glycine-proline based linker. These linkers comprise glycine and proline residues and may be between 3 and 30, 10 and 30, and 3 and 20 amino acids in length. Examples of such linkers include GPG, GPGPGPG (SEQ ID NO: 90) and GPGPGPGPGPG (SEQ ID NO: 91). In some embodiments, the linker is a proline-alanine based linker. These linkers comprise proline and alanine residues and may be between 3 and 30, 10 and 30, 3 and 20 and 6 and 18 amino acids in length. Examples of such linkers include PAPAPA (SEQ ID NO: 92), PAPAPAPAPAPA (SEQ ID NO: 93) and PAPAPAPAPAPAPAPAPA (SEQ ID NO: 94). In some embodiments, the linker is a glycine-serine based linker. These linkers comprise glycine and serine residues and may be between 8 and 50, 10 and 30, and 10 and 20 amino acids in length. Examples of such linkers include GSGSGSGSGS ((GS)₅; SEQ ID NO: 95), GSGSGSGSGSGS ((GS)₆; SEQ ID NO: 96), GSGSGSGSGSGSGSGSGSGS ((GS)₁₀; SEQ ID NO: 97), GGGGSGGGGSGGGGS ((G₄S)₄; SEQ ID NO: 98), GGGGSGGGGSGGGGSGGGGSGGGGS ((G₄S)₅; SEQ ID NO: 99), and GGGGSGGGGSGGGSG (SEQ ID NO: 100). In exemplary embodiments, the linker does not contain any Asp-Lys (DK) pairs.

Nucleic Acid-Protein Fusion Technology

One way to rapidly make and test FBS domains with specific binding properties is the nucleic acid-protein fusion technology of Adnexus, a Bristol-Myers Squibb Company. Such in vitro expression and tagging technology, termed Profusion, that exploits nucleic acid-protein fusions (RNA- and DNA-protein fusions) may be used to identify novel polypeptides and amino acid motifs that are important for binding to proteins. Nucleic acid-protein fusion technology is a technology that covalently couples a protein to its encoding genetic information. For a detailed description of the RNA-protein fusion technology and fibronectin-based scaffold protein library screening methods see Szostak et al., U.S. Pat. Nos. 6,258,558; 6,261,804; 6,214,553; 6,281,344; 6,207,446; 6,518,018; PCT Publication Nos. WO 00/34784; WO 01/64942; WO 02/032925; and Roberts et al., Proc Natl. Acad. Sci., 94:12297-12302 (1997), herein incorporated by reference.

### HSA Proteins

The human serum albumin molecule, the most abundant serum protein, is comprised of three homologous, primarily helical domains identified as domain I, domain II and domain III, each of which contains two subdomains, namely IA, IB, IIA, IIB and IIIA, IIIB Unlike the whole albumin protein, these fragments generally have particular binding affinities and act as binding sites for different ligands. Albumins have been characterized from many species, including human, pig, mouse, rat, rabbit and goat, and they share a high degree of sequence and structural homology. The plasma half-life of HSA is approximately 19 days.

Albumin binds in vivo to its receptor, the neonatal Fc receptor (FcRn) “the Brambell receptor” and this interaction is known to be important for the plasma half-life of albumin. FcRn is a membrane bound protein, expressed in many cell and tissue types. FcRn HSA been found to salvage albumin from intracellular degradation (Roopenian, D. et al., Nat. Rev. Immunol., 7:715-725 (2007)).

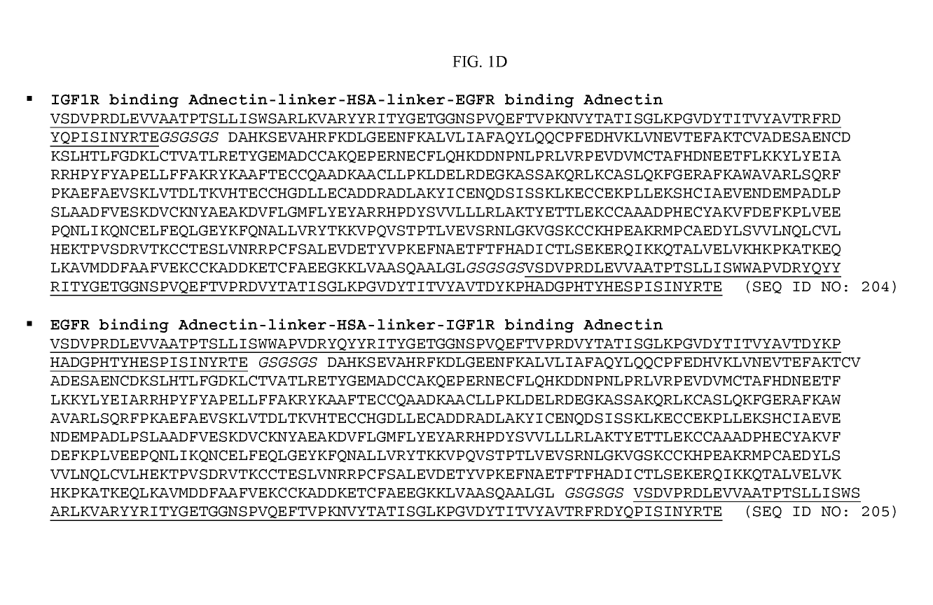

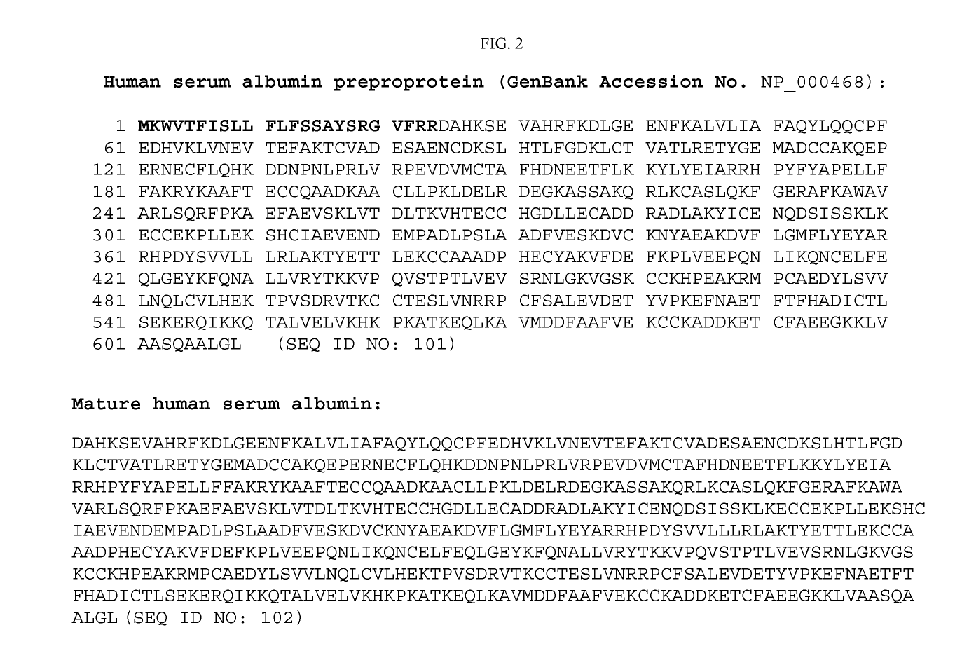

Human serum albumin (HSA) is a polypeptide of 585 amino acids, the sequence of which can be found in Peters, T., Jr., All About Albumin: Biochemistry, Genetics and Medical Applications, pp. 10, Academic Press, Inc., Orlando (1996) (ISBN 0-12-552110-3). The mature wild-type form of HSA consists of the following amino acid sequence (corresponding to amino acids 25-585 of GENBANK® Accession No. NP_000468 and also shown in FIG. 2):

(SEQ ID NO: 102)

DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL

Wild-type HSA with its natural signal sequence is shown in FIG. 2 and is set forth as SEQ ID NO: 101.

As used herein “human serum albumin” is used interchangeably with “HSA” and refers to wild type HSA (having SEQ ID NO: 101 or 102) and to biologically active variants thereof, such as variants that improve one or more properties of a protein to which it is linked. The one or more properties may be selected from the group consisting of improved pharmacokinetics, increased shelf-life, increased solubility, increased affinity for the target and increased biological activity. Biologically active variants of HSA include, e.g., variants that have similar, equal, improved or reduced binding affinity to the FcRn receptor, such that they have the ability to be recycled through the lysozome. The term “HSA” includes HSA with a signal sequence and HSA without signal sequence (i.e., mature HSA).

HSA proteins for use in the fusion proteins described herein include proteins comprising, consisting or consisting essentially of an amino acid sequence that is at least about 70%, 75%, 80%, 85%, 90%, 95%, 97%, 98% or 99% identical to wild-type HSA having SEQ ID NO: 101 or 102. HSA proteins for use herein may comprise about, at least or at most 1, 2, 3, 4, 5, 6, 7, 8, 9, 10, 15, 20, 25, 30, 35, 40, 45 or 50 amino acid changes relative to SEQ ID NO: 101 or 102. HSA proteins may comprise about, at least or at most 1-5, 1-10, 1-15, 1-20, 1-25, 1-30, 1-35, 1-40, 1-45 or 1-50 amino acid changes relative to SEQ ID NO: 101 or 102, and these changes may be located within the protein, at its N-terminus and/or at its C-terminus Amino acid changes include amino acid substitutions (e.g., conservative substitutions), additions or deletions. HSA proteins or moieties that differ from HSA proteins comprising the amino acid sequence SEQ ID NO: 101 or 102 are preferably biologically active and provides at least one improved property to the moiety with which it is associated (e.g., a FBS moiety) as compared to the unmodified moiety to which it is associated. An improved property may be improved pharmacokinetics (PK), increased half-life, increased shelf-life, increased solubility, increased affinity for the target, increased biological activity or reduced immunogenicity. HSA moieties that differ from HSA moieties comprising the amino acid sequence SEQ ID NO: 101 or 102 (i.e., HSA variants) may have at least one biological activity or characteristic that differs from that of an HSA moiety comprising the amino acid sequence SEQ ID NO: 101 or 102. For example, an HSA variant may provide a longer or shorter half-life (e.g., serum half-life) to a protein to which it is linked relative to a wild type HSA (having SEQ ID NO: 101 or 102). In certain embodiments, an HSA variant provides a property to a protein to which it is associated that is that is an improvement over the unmodified protein to which it is associated, but less of an improvement relative to wild-type HSA. In certain embodiments, the half-life of a fusion protein comprising an HSA variant and another protein is longer or shorter by at least 50%, 100% (2 fold), 3 fold, 4 fold, 5 fold, 10 fold or more relative to wild-type HSA linked to the same protein (and in the same conformation). For example, an HSA variant may provide a half-life to a protein to which it is associated that is 1-5 days, 1-10 days, 5-10 days, 5-15 days, 10-15 days, 10-20 days, 10-30 days, 20-30 days, 25-30 days, 25-40 days, 30-40 days or more than 40 days.

Variants of HSA that may be used in fusion proteins include naturally-occurring variants and non-naturally-occurring variants. The following naturally-occurring variants are known and may be used to the extent that they improve at least one characteristic of a protein to which it is linked. Some of the variants have been reported to reduce plasma half-life, but these may still be useful in fusion proteins with FBS proteins if they improve at least one property of an FBS protein to which it is linked relative to a unmodified FBS protein. To the extent that a variant HSA reduces at least one biological property of a protein to which it is linked to an undesirable level, such variant may be specifically excluded from HSA moieties fused to FBS moieties.

Exemplary Natural Variants:

D494N: lower plasma half-life (Peach, R. J. et al., Biochem. Biophys. Acta, 1097:49-54 (1991)). This substitution generated an N-glycosylation site in this variant, which is not present in the wild-type albumin;

K541 E and K560E have reduced half-life; E501 K and E570K have increased half-life; and K573E HSA essentially no effect on half-life (Iwao et al., B.B.A. Proteins and Proteomics, 1774:1582-1590 (2007)); and

Deletion of the C-terminal 175 amino acids at the carboxy terminus HSA a reduced half-life (Andersen et al., Clinical Biochemistry, 43:367-372 (2010)).

Additional natural variants include E505K (Galliano et al., Biochim. Biophys. Acta 1225, 27-32 (1993)); K536E (Minchiotti et al (1990)); K574N (Minchiotti et al., Biochim. Biophys. Acta 916, 41 1-418 (1987)); D550G (Takahsahi et al., Proc. Natl. Acad. Sci. USA, 84:4413-4417 (1987)); D550A (Carlson et al., Proc. Nat. Acad. Sci. USA, 89:8225-8229 (1992)). In addition, 77 albumin variants of which 25 have mutations in domain III are disclosed in Otagiri et al., Biol. Pharm. Bull., 32(4):527-534 (2009). Additional variants that may be used include those having one or more of the following amino acid substitutions: L407A; L408V; V409A; R410A; K413Q; and K414Q (described in WO 95/23857 and WO 2009/058322, which are specifically incorporated by reference herein for the description of all HSA variants).

Other HSA variants that may be used include those consisting of fragments of wild-type HSA (optionally with additional amino acid changes), such as the following fragments: a fragment consisting of amino acids 1-194, 195-387, 388-585, 1-387, 195-585, 1-105, 120-194, 195-291, 316-387, 388-491 or 512-585 of SEQ ID NO: 102 (described in WO 2009/058322, which is specifically incorporated by reference herein for the description of HSA variants).

Variants of HSA that may be used may also include portions that are derived from a non-human animal serum albumin protein, e.g., a protein from macaque albumin, rabbit albumin, mouse albumin, sheep albumin, goat albumin, chimpanzee albumin, hamster albumin, guinea pig albumin, rat albumin, cow albumin, horse albumin, donkey albumin, dog albumin, chicken albumin, or pig albumin. Variants may comprise one portion from a first non-human animal and a second portion from another non-human animal. In certain embodiments, the N-terminal portion of a variant comprises amino acids 1 to 565, 566, 567, 568, 569, 570, 571, 572, 573, 574 or 575 of mature HSA (SEQ ID NO: 102). Exemplary variants comprise or consist or consist essentially of SEQ ID NO: 20, SEQ ID NO: 21, SEQ ID NO: 22 or SEQ ID NO: 23 of WO 2012/059486, which is specifically incorporated by reference herein.

Variants may also comprise, consist or consist essentially of HSA domain 1 (amino acids 1-194 of SEQ ID NO: 102), domain 2 (amino acids 195-387 of SEQ ID NO: 102), domain 3 (amino acids 388-585 of SEQ ID NO: 102), domains 1 and 2 (amino acids 1-387 of SEQ ID NO: 102), domains 2 and 3 (amino acids 195-585 of SEQ ID NO: 102), domains 1 and 3 (amino acids 1-194 and 388-585 of SEQ ID NO: 102) or 2 copies of domain 3 (twice amino acids 388-585 of SEQ ID NO: 102), as described in WO 2011/124718 and US 2007/0048282, both of which are specifically incorporated by reference herein. Domain I may or may not start at amino acid 1 and may or may not end at any of amino acids 192, 193, 194, 195, 196 or 197, preferably any of amino acids 192, 194 or 197. Domain II may or may not start at amino acid 189, 190, 191, 192 or 193, preferably any of amino acids 189, 192 or 193, and may or may not end at amino acid 382, 383, 384, 385, 386 or 387, preferably any of amino acids 382, 285 or 387. Domain III may or may not start at amino acid 381, 382 or 383, preferably amino acid 381 or 383, and may or may not end at amino acid 585. Domain III may comprise one or more amino acid changes at one or more of the following positions: 417, 440, 464, 490, 492, 493, 494, 495, 496, 499, 500, 501, 503, 504, 505, 506, 510, 535, 536, 537, 538, 540, 541, 542, 550, 573, 574, 575, 577, 578, 579, 580, 581, 582 and 584. Exemplary changes include one or more of the following amino acid changes: Q417A; H440Q; H464Q; A490D; E492G; T,P,H; V493P,L; D494N,Q,A,E,P; E495Q,A; T496A; P499A; K500E,G,D,A,S,C,P, H,F,N,W, T,M,Y,V,Q,L,I,R; E501A,P,Q; N503K,D,H; A504E; E505K, D; T506F, S; H510Q; H535Q; K536A; P537A; K538A,H; T540S; K541A,D,G,N,E; E542P,D; D550N; K573Y,W,P,H,F,V,I,T,N,S,G,M,C, A,E,Q,R, L,D; K574N; Q580K; L575F; A577T,E; A578R,S; S579C,T; Q580K; A581D; A582T; G584A of SEQ ID NO: 102. Variants may comprise changes at one or more of the following amino acids 492, 503, 542, 550, 573, 574, 580, 581, 582 or 584 in SEQ ID NO: 102. Exemplary variants comprise one or more of the following changes: E492G; N503K,H; D550E;

K573Y,W,P,H,F,V,I,T,N,S,G,M,C,A,E,Q,R,L or a D; K574N; and Q580K residue. Variants may comprise one or more of the following changes: E492G; K573 A or P. Other variants include changes at amino acids 492 and 503 of SEQ ID NO: 102. A variant may also comprise E492G; N503H or K and K573A or P. Any other amino acid change that is described in WO 2011/124718 is specifically incorporated by reference herein. These amino acid changes may be present in embodiments, in which domain 3 is used alone (i.e., as the only portion of HSA), or in which domain 3 is only one portion of an HSA.

Exemplary amino acid changes to HSA that increase its half-life include changes to E492, N503, D550 and/or K573 in HSA. For example, the amino acid residue in the position corresponding to E492 in HSA may be substituted with a G residue, the amino acid in the position corresponding to N503 in HSA may be substituted with a H or K residue, the amino acid in the position corresponding to D550 in HSA may be substituted with a E residue and the amino acid in the position corresponding to K573 in HSA may be substituted with an A or a P residue. Exemplary variants with an enhanced half-life include: HSA in which E492 is changed to a G residue and the amino acid corresponding to K573 is changed to an A or a P residue. Other variants have the following substitutions: E492H, E501P, N503H, E505D, T5065, T540S and/or K541E (WO 2011/124718, which is specifically incorporated by reference herein).

Other mutations increasing hFcRn affinity and therefore half-life include V418M, T420A, E505G and V547A. An HSA molecule comprising all 4 of these mutations has a Kd at pH 6.0 that is at least 300 fold lower than that of wild-type HSA (Schmidt et al., Structure, 21:1966 (2013)). The following HSA mutants described in Schmidt et al. may also be fused to an adnectin: V418M/T420A/V547A; E505G/V547A; V418M/T420A/E505R; V418M/T420A/E505G; V547A; V418M/T420A; E505G; E505R; T420A; V418M; V418M/T420A/V424I/N429D/E505R; V418M/T420A/M444V; A469V/T467M; E505G/A552T; V424I; E492G; E505K; N429D; M444B/A469V; T467M; and A552T (see Table S2 of Schmidt et al.).

Any other variant of HSA that improves at least one property of a protein to which it is linked, e.g., serum half-life, may be used in a fusion protein with an FBS protein. For Example, HSA may have a mutation at C34, e.g., C34S, C34A or C34E.

Other variants that may be used are described, e.g., in EP 322094, WO 1990/013653, WO 1997/024445, U.S. Pat. No. 6,165,470, WO 1993/01599, WO 2001/079271, and WO 2003/059934, all of which are specifically incorporated by reference herein.

### HSA Binding Moieties

Provided herein are FBS proteins linked to an HSA binding moiety. An “HSA binding moiety” refers to a moiety that specifically binds to HSA, which, when linked to a protein, e.g., an FBS protein, improves at least one property of the protein (e.g., serum half-life) relative to the unmodified protein. “HSA binding moiety” includes eukaryotic, such as animal (e.g., mammalian) and prokaryotic (e.g., bacterial) proteins. In certain embodiments, an HSA binding moiety is a bacterial receptor protein or an HSA binding portion thereof. In certain embodiments, an HSA binding moiety is a protein comprising a three-helix bundle structure. In certain embodiments, an HSA binding moiety is a bacterial receptor protein comprising a three-helix bundle domain. In certain embodiments, an HSA binding moiety is domain GA1, GA2, or GA3 of protein G from Streptococcus strain G148. In a preferred embodiment, an HSA binding moiety comprises, consists of, or consists essentially of the wild-type 46 amino acid long three-helix bundle domain of Streptococcal protein G (SpG) having the following amino acid sequence:

LAEAKVLANRELDKYGVSDYYKNLINNAKTVEGVKALIDEILAALP
(SEQ ID NO: 103; referred to as a “wild-type
ABD”).

In certain embodiments, additional sequences of protein G may be included, e.g., at least 1, 1, 2, 3, 4, 5, 6, 7, 8, 9 or 10 contiguous amino acids on the N-terminal and/or the C-terminal end of a peptide consisting of SEQ ID NO: 103. For example, the following wild-type ABD sequences may be used:

(SEQ ID NO: 104)

SLAEAKVLANRELDKYGVSDYYKNLINNAKTVEGVKALIDEILAALP

(SEQ ID NO: 105)

NSLAEAKVLANRELDKYGVSDYYKNLINNAKTVEGVKALIDEILAALP

(SEQ ID NO: 106)

ANSLAEAKVLANRELDKYGVSDYYKNLINNAKTVEGVKALIDEILAALP

(SEQ ID NO: 107)

DANSLAEAKVLANRELDKYGVSDYYKNLINNAKTVEGVKALIDEILAALP

(SEQ ID NO: 210)

VDANSLAEAKVLANRELDKYGVSDYYKNLINNAKTVEGVKALIDEILAAL
P

(SEQ ID NO: 108)

AVDANSLAEAKVLANRELDKYGVSDYYKNLINNAKTVEGVKALIDEILAA
LP

(SEQ ID NO: 109)

EAVDANSLAEAKVLANRELDKYGVSDYYKNLINNAKTVEGVKALIDEILA
ALP

(SEQ ID NO: 110)

DEAVDANSLAEAKVLANRELDKYGVSDYYKNLINNAKTVEGVKALIDEIL
AALP

(SEQ ID NO: 111)

HDEAVDANSLAEAKVLANRELDKYGVSDYYKNLINNAKTVEGVKALIDEI
LAALP

(SEQ ID NO: 112)

QHDEAVDANSLAEAKVLANRELDKYGVSDYYKNLINNAKTVEGVKALIDE
ILAALP

In certain embodiments, an ABD sequence does not comprise a “V” between the A and D in any of SEQ ID NOs: 108-112 or N-terminal in SEQ ID NO: 210 (Kraulis et al., FEBS Lett., 378:190 (1996)).

Variants of wild-type ABD (having one of SEQ ID NOs: 103-112) may also be used, provided that they improve at least one property of a protein to which it is linked, such as serum half-life, stability, solubility, and/or Kd, biological activity, and/or reduces immunogenicity. Variants may comprise an amino acid sequence that is at least 70%, 75%, 80%, 85%, 90%, 95%, 97%, 98%, or 99% identical to any one of SEQ ID NOs: 103-112 and 210. Variants may also comprise about, at least or at most 1, 2, 3, 4, 5, 6, 7, 8, 9, 10 or more amino acid alterations, such as amino acid substitutions, additions or deletions, relative to any of SEQ ID NOs: 103-112 and 210.

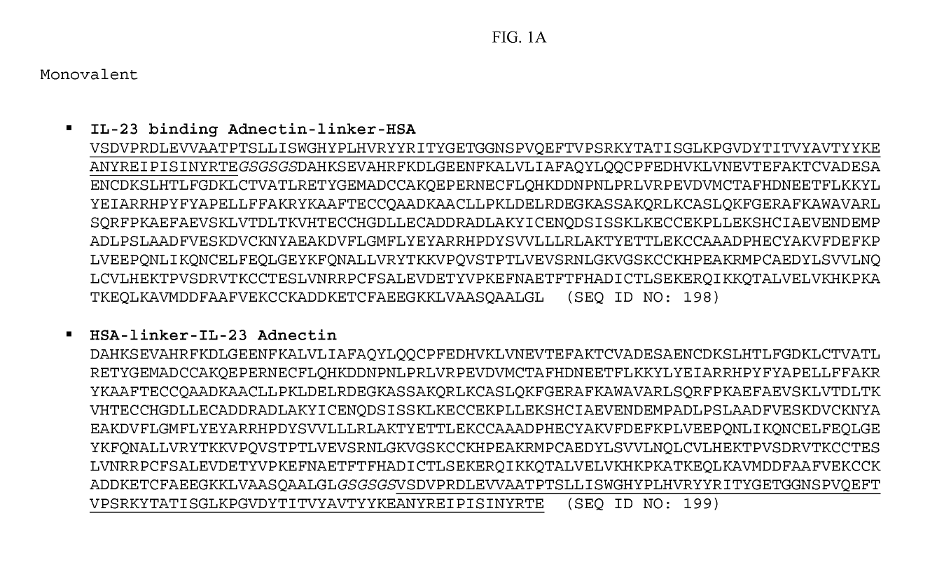

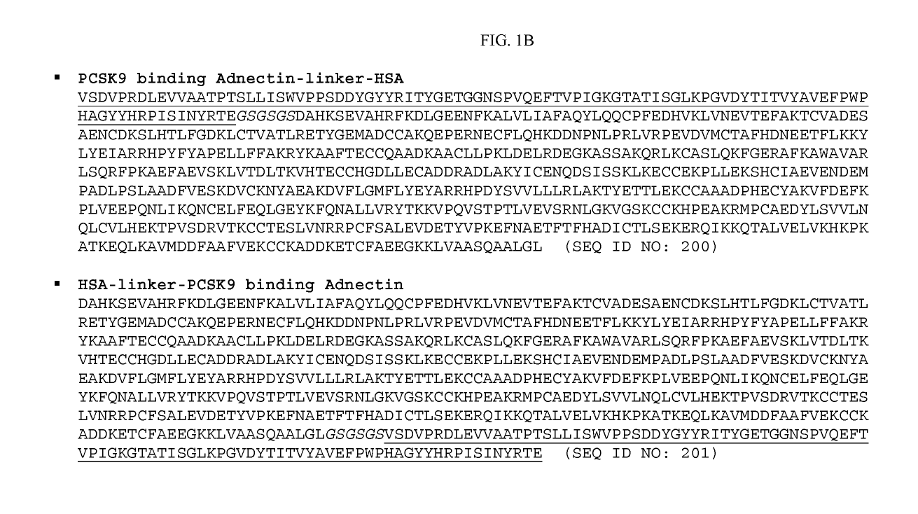

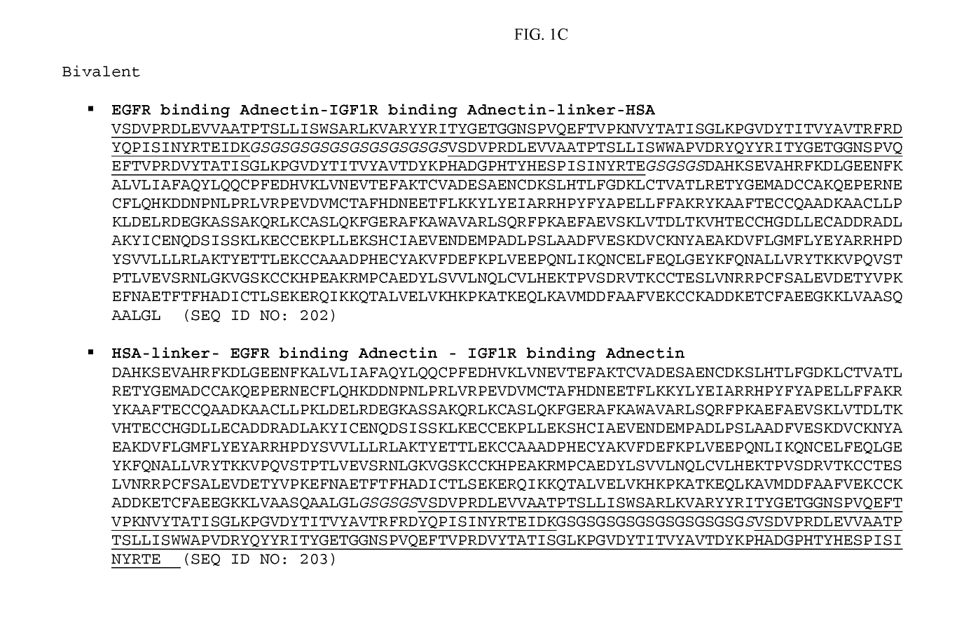

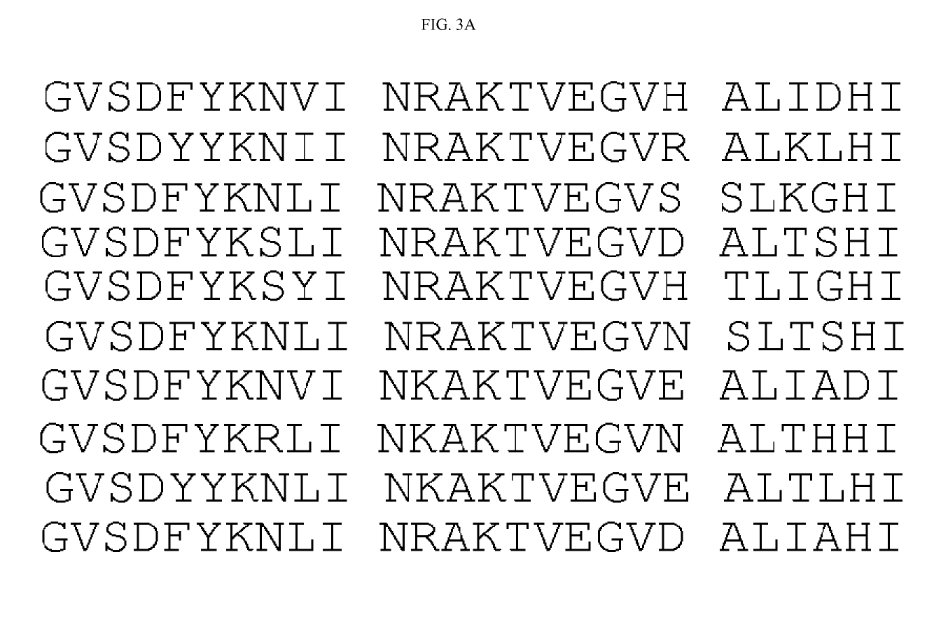

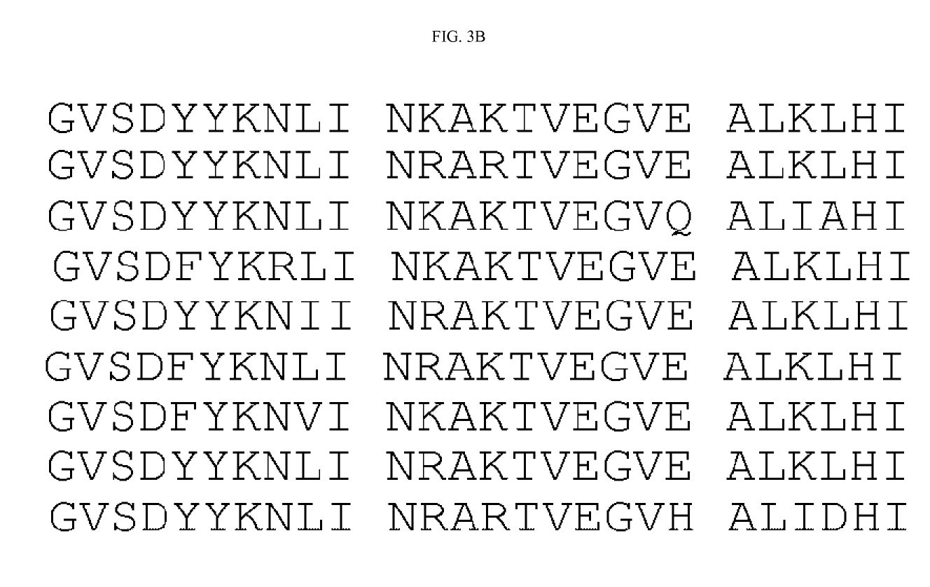

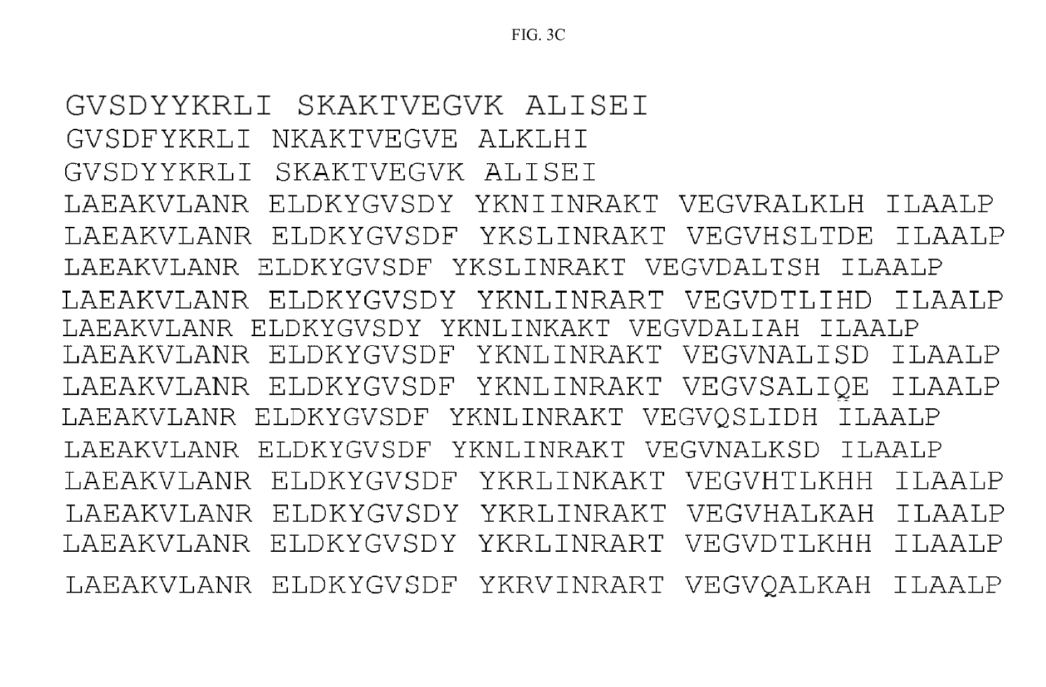

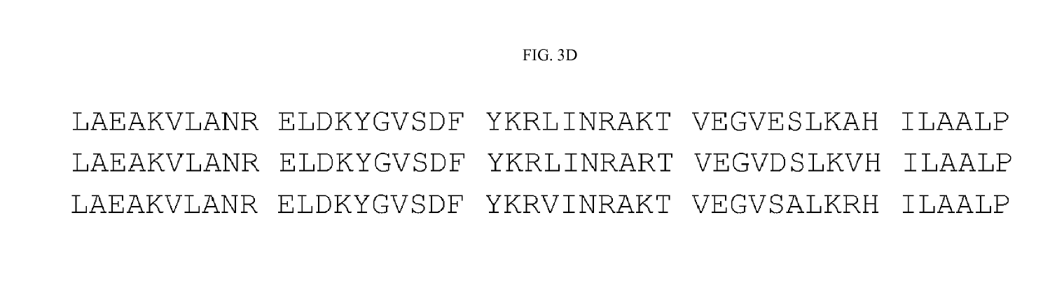

Variants of wild-type ABD that may be used include any of SEQ ID NOs: 1-257 and 258-514, shown in FIG. 1 of WO 2009/1016043. Exemplary variants that may be used include those shown in FIGS. 3A-D (SEQ ID NOs: 113-150).

In certain embodiments, an HSA binding protein comprises one of the following amino acid sequences:

(SEQ ID NO: 151)

DICLPRWGCLW

(SEQ ID NO: 152)

DLCLRDWGCLW

(SEQ ID NO: 153)

DICLARWGCLW

In one embodiment, a fusion protein comprising an FBS domain and an ABD comprises the following sequence:

(SEQ ID NO: 154)

MGVSDVPRDLEVVAATPTSLLISWVPPSDDYGYYRITYGETGGNSPVQEF
TVPIGKGTATISGLKPGVDYTITVYAVEFPWPHAGYYHRPISINYRTGSG
SGSQHDEAVDANSLAEAKVLANRELDKYGVSDYYKNLINNAKTVEGVKAL
IDEILAALP.

SEQ ID NO: 154 comprises the amino acid sequence of a PCSK9 binding adnectin. As shown in Example 2, a fusion protein comprising SEQ ID NO: 154 and an ABD domain binds efficiently to PCSK9 and has a long serum half-life in Cynomolgus monkeys.

Other bacterial HSA binding proteins than protein G from Streptococcus may also be used for fusing to an FBS protein. For example, HSA binding moieties from the Streptococcus proteins PAB, PPL, MAG and ZAG may also be used (Johansson et al., J. Mol. Biol., 266:859 (1997) and Johannson et al., J. Biol. Chem., 277:8114 (2002)). HSA binding moieties in these proteins, which binding moieties comprise the three-helix protein domain responsible for albumin binding, have been referred to as “GA module” (protein G-related Albumin binding module) in Johannson et al. Artificial variants of the GA module have also been created, and may be used in fusion proteins with an FBS moiety. Exemplary artificial variants of the GA module are described in Rozak et al., Biochem., 45:3263 (2006).

Additional bacterial HSA binding moieties that may be used in fusion proteins comprising an FBS moiety include HSA binding moieties present in the family of Streptococcal proteins referred to as the “M proteins”, e.g., M1/Emm1, M3/Emm3, M12/Emm12, EmmL55/Emm55, Emm49/EmmL49, and H (see, e.g., Table 2 in Navarre et al., MMBR, 63:174 (1999)).

Other serum albumin binding domains or proteins that may be used to improve at least one property of a protein (e.g., an FBS domain) to which it is linked include peptides, antibodies or antigen binding portions thereof or other molecules that bind to serum albumin. For example, an FBS domain may be linked to albumin binding peptides (ABP), such as peptides isolated by screening random peptide libraries for those peptides that bind to HSA. Exemplary peptides are described, e.g., in Nguyen et al., Prot. Eng. Designs Sel., 19:29 (2006). Other albumin binding domains include single domain antibodies, e.g., Camelidae VHHs antibodies or Nanobodies (or AlbudAb), that bind to HSA, as described, e.g., in U.S. Application Publication No. 2007/0178082. Exemplary sequences described therein that may be fused to FBS domains are provided as SEQ ID NOS: 1 to 4, and 28 to 40 in U.S. Application Publication No. 2007/0178082. Single domain antibodies that bind to HSA and may be linked to FBS domains include those described in WO 04/041865. For example, the following humanized single domain antibodies (or CDRs thereof) described in U.S. Application Publication No. 2007/0269422 may be fused to FBS proteins:

ALB3 (ALB1 HUM1)

(SEQ ID NO: 155)

EVQLVESGGGLVQPGGSLRLSCAASGFTFRSFGMSWVRQAPGKEPEWVSS
ISGSGSDTLYADSVKGRFTISRDNAKTTLYLQMNSLKPEDTAVYYCTIGG
SLSRSSQGTQVTVSS;
ALB4 (ALB1 HUM2)

(SEQ ID NO: 156)

EVQLVESGGGLVQPGGSLRLSCAASGFTFSSFGMSWVRQAPGKEPEWVSS
ISGSGSDTLYADSVKGRFTISRDNAKTTLYLQMNSLKPEDTAVYYCTIGG
SLSRSSQGTQVTVSS;
ALBS (ALB1 HUM3)

(SEQ ID NO: 157)

EVQLVESGGGLVQPGGSLRLSCAASGFTFRSFGMSWVRQAPGKGLEWVSS
ISGSGSDTLYADSVKGRFTISRDNAKTTLYLQMNSLKPEDTAVYYCTIGG
SLSRSSQGTQVTVSS;
ALB6 (ALB1 HUM1)

(SEQ ID NO: 158)

EVQLVESGGGLVQPGNSLRLSCAASGFTFRSFGMSWVRQAPGKGLEWVSS
ISGSGSDTLYADSVKGRFTISRDNAKTTLYLQMNSLKPEDTAVYYCTIGG
SLSRSSQGTLVTVSS;
ALB7 (ALB1 HUM2)

(SEQ ID NO: 159)

EVQLVESGGGLVQPGNSLRLSCAASGFTFRSFGMSWVRQAPGKGLEWVSS
ISGSGSDTLYADSVKGRFTISRDNAKTTLYLQMNSLRPEDTAVYYCTIGG
SLSRSSQGTLVTVSS;
ALB8 (ALB1 HUM3)

(SEQ ID NO: 160)

EVQLVESGGGLVQPGNSLRLSCAASGFTFSSFGMSWVRQAPGKGLEWVSS
ISGSGSDTLYADSVKGRETISRDNAKTTLYLQMNSLRPEDTAVYYCTIGG
SLSRSSQGTLVTVSS;
ALB9 (ALB1 HUM4)

(SEQ ID NO: 161)

EVQLVESGGGLVQPGNSLRLSCAASGFTFSSFGMSWVRQAPGKGLEWVSS
ISGSGSDTLYADSVKGRETISRDNAKNTLYLQMNSLRPEDTAVYYCTIGG
SLSRSSQGTLVTVSS;
and
ALB10 (ALB1 HUM5)

(SEQ ID NO: 162)

EVQLVESGGGLVQPGNSLRLSCAASGFTFSSFGMSWVRQAPGKGLEWVSS
ISGSGSDTLYADSVKGRFTISRDNAKNTLYLQMNSLRPEDTAVYYCTIGG
SLSRSGQGTLVTVSS.

Other single domain antibodies that may be used are described in WO 91/01743, WO 01/45746 and WO 02/076489, all of which are incorporated by reference herein.

Other HSA binding domains that may be used include albumin binding antibody fragments and albumin binding domain antibodies (dAbs) (see, e.g., Holt et al., Prot. Eng. Design Sel., 21:283 (2008) and WO 06/059106). Non-proteinaceous albumin-binding moieties may also be used as moieties that bind HSA. For example, an FBS moiety may be linked to one or more long-chain fatty acid chain or they may be acylated. An adnectin binding to HSA (as described, e.g., in WO 2011/140086) may also be linked to an FBS domain.

Other moieties that may be used to improve at least one property of a protein (e.g., an FBS domain) to which it is linked include generally any modification to a protein that increases its hydrodynamic radius and/or increases its recycling through the FcRn receptor. Exemplary moieties that may be linked to a protein include carbohydrates, polymers, and polypeptides. In certain embodiments, PEG or a recombinant PEG mimetic is used. Exemplary PEG mimetics include natural, biodegradable and hydrophilic polymers, such as polysialic acid and hydroxyethyl starch. PEG mimetics also include long flexible polypeptides, e.g., a glycine-rich homo-amino-acid polymer (HAP) having, e.g., from 100-200 amino acids and composed of multimers of the (G₄S) may be used (Schlapschy et al., Prot. Eng. Design Sel., 20:273 (2007)). Another PEG mimetic is PAS, which is a recombinant PEG mimetics based on the three amino acids proline, alanine, and serine. A flexible, hydrophilic, glycine-rich (but unstructured) repetitive polymer described in Volker et al. (2009) Amunix.com/Technology.html may also be used. These polymers are referred to as half-life extension (XTEN) polymers.

Carbohydrate conjugates (e.g., hydroxyethyl starch (HES)), glycosylation (e.g., introduction of N- or O-glycans), polymeric sugar, polysialic acid conjugates, and fatty acid conjugates may also be used to modify an FBS domain to improve at least one property of the FBS domain. For example, polysialic acid polymers, e.g., colominic acid, may be linked to an FBS domain. Polysialic acid polymers are described, e.g., in Fernandez et al., 217:215 (2001), Jain et al., Biochim. Biophys. Acta, 1622:42 (2003) and Constantinou et al., Bioconjug. Chem., 19:643 (2008). Fibronectin based scaffold domains may also be glycosylated, e.g., by changing one or more amino acids in the amino acid sequence to form a glycosylation site or by adding one or more amino acids, e.g., at the N- or C-terminus of the protein to form a glycosylation site. Expression in a mammalian system will then allow the FBS protein to become glycosylated. Any other PK moiety or modification described in Kontermann et al. (Current Opinion in Biotechnology, 22:868-876 (2011); herein incorporated by reference) may be linked to an FBS domain. Any of the moieties described herein that improve at least one property of a protein to which it is linked may be linked to any of the FBS moieties described herein.

For purposes of clarity, when referring herein to moiety 1 being “linked” or “connected” to moiety 2, the linkage or connection may be direct, i.e., the two moieties are fused together without any intervening amino acid(s), or indirectly, i.e., the two moieties are fused together with at least one additional amino acid or linker between the two moieties. The terms “linked” or “connected” when referring to two moieties do not imply a particular arrangement of two moieties, i.e., one or the other moiety may be N-terminal to the other one.

### Linkers for Connecting an FBS Moiety to an HSA or HSA Binding Moiety

Any linker may be used for covalently linking an FBS moiety to HSA or an HSA binding moiety, provided that the linker allows the fusion protein comprising the FBS moiety and the HSA or HSA binding moiety to properly fold and be biologically active. For example, the fusion protein should be able to bind efficiently to its target and have an improved property, e.g., a long half-life in serum, relative to the unmodified FBS protein. A linker is also preferably essentially not immunogenic and not reactive with other proteins (i.e., chemically inert).

A linker may be from 1-6, 1-10, 1-15, 1-20, 1-25, 1-30, 1-35, 1-40, 1-45, 1-50, 5-10, 5-15, 5-15, 5-20, 5-25, 5-30, 5-35, 5-40, 5-45, or 5-50 amino acids long.

Exemplary linkers may comprise, consist of, or consist essentially of GS linkers, e.g., (GS)₁, (GS)₂, (GS)₃, (GS)₄, (GS)₅, (GS)₆, (GS)₇, (GS)₈, (GS)₉ or (GS)₁₀. Linkers may also comprise, consist of, or consist essentially of G4S linkers, e.g., (G₄S)₁, (G₄S)₂, (G₄S)₃, (G₄S)₄ or (G₄S)₅. Linkers that are used for linking two FBS proteins (as further described herein) may also be used (see, e.g., SEQ ID NOs: 90-100). A preferred linker is (GS)₃.

Additional linkers that may be used include the following:

(SEQ ID NO: 163)

SCSVADWQMPPPYVVLDLPQETLEEETPGAN;

(SEQ ID NO: 164)

SCCVADWQMPPPYVVLDLPQETLEEETPGAN;

(SEQ ID NO: 165)

DWQMPPPYVVLDLPQETLEEETPGAN;

(SEQ ID NO: 166)

SCCVADWQMPPPYVVLDLPQETLEEETPGAN;

(SEQ ID NO: 167)

YLAMTPLIPQSKDENSDDYTTFDDVGS;

(SEQ ID NO: 168)

ELDVCVEEAEGEAPW;

(SEQ ID NO: 169)

ELQLEESCAEAQDGELDG;

(SEQ ID NO: 170)

EGEVSADEEGFEN;

(SEQ ID NO: 171)

KPTHVNVSVVMAEVDGTCY;

(SEQ ID NO: 172)

KPTHVNVSVVMAEVDGTCY;

(SEQ ID NO: 173)

YVTDHGPMK;

(SEQ ID NO: 174)

PTLYNVSLVMSDTAGTCY;

(SEQ ID NO: 175)

SXSVADWQMPPPYVVLDLPQETLEEETPGAN,
wherein X is serine, alanine or glycine;

(SEQ ID NO: 176)

SXXVADWQMPPPYVVLDLPQETLEEETPGAN,
wherein each X is independently selected
from serine, alanine or
glycine;

(SEQ ID NO: 177)

SXXVADWQMPPPYVVLDLPQETLEEETPGAN,
wherein each X is independently selected
from serine, alanine or glycine;

(SEQ ID NO: 178)

ELDVXVEEAEGEAPW,
wherein X is serine, alanine or glycine;

(SEQ ID NO: 179)

ELQLEESXAEAQDGELDG,
wherein X is serine, alanine or glycine;

(SEQ ID NO: 180)

KPTHVNVSVVMAEVDGTXY,
wherein X is serine, alanine or glycine;

(SEQ ID NO: 181)

KPTHVNVSVVMAEVDGTXY,
wherein X is serine, alanine or glycine;
and

(SEQ ID NO: 182)

PTLYNVSLVMSDTAGTXY,
wherein X is serine, alanine or glycine;

(SEQ ID NO: 183)

DKTHTCPPCPAPELLG;

(SEQ ID NO: 184; core hinge region underlined)

EPKSSDKTHTCPPCPAPELLGGPS;

(SEQ ID NO: 185; core hinge region underlined)

EPKSSDKTHTCPPCPAPELLGGSS;

(SEQ ID NO: 186; core hinge region underlined)

EPKSSGSTHTCPPCPAPELLGGSS;

(SEQ ID NO: 187; core hinge region underlined)

DKTHTCPPCPAPELLGGPS;

(SEQ ID NO: 188, core hinge region underlined)

DKTHTCPPCPAPELLGGSS;

(SEQ ID NO: 189)

AGGGGSG;

(SEQ ID NO: 190)

AGGGGSGG;

(SEQ ID NO: 191)

QPDEPGGS;

(SEQ ID NO: 192)

ELQLEESAAEAQDGELD;

(SEQ ID NO: 193)

TVAAPS;

(SEQ ID NO: 194)

QPDEPGGSG;

(SEQ ID NO: 195)

ELQLEESAAEAQDGELDG;
and

(SEQ ID NO: 196)

TVAAPSG.

### Exemplary Fusion Proteins Comprising an FBS Moiety and an HSA Moiety

Provided herein are fusion proteins (e.g., target binding fusion proteins) comprising an amino acid sequence encoding an FBS moiety that binds specifically to a given target (e.g., a target protein) and an amino acid sequence that encodes an HSA moiety. The FBS moiety may be located C-terminally or N-terminally to the HSA moiety. The FBS moiety may be linked directly (i.e., without one or more intervening amino acids) or indirectly to the HSA moiety. An “indirect linkage” between two protein moieties refers to a linkage between two protein moieties that comprises at least one intervening amino acid. In one embodiment, a fusion protein comprises an FBS moiety that is covalently linked at its C-terminus to the N-terminus of a linker, which linker is covalently linked at its C-terminus to the N-terminus of an HSA moiety. In another embodiment, a fusion protein comprises an HSA moiety that is covalently linked at its C-terminus to the N-terminus of a linker, which linker is covalently linked at its C-terminus to the N-terminus of an FBS moiety. In another embodiment, a fusion protein comprises a first FBS moiety that is covalently linked at its C-terminus to the N-terminus of a second FBS moiety, which second FBS moiety is covalently linked at its C-terminus to the N-terminus of a linker, which linker is covalently linked at its C-terminus to the N-terminus of an HSA moiety. In another embodiment, a fusion protein comprises a first FBS moiety that is covalently linked at its C-terminus to the N-terminus of a first linker, which first linker is covalently linked at its C-terminus to the N-terminus of a second FBS moiety, which second FBS moiety is covalently linked at its C-terminus to the N-terminus of a second linker, which second linker is covalently linked at its C-terminus to the N-terminus of an HSA moiety. In another embodiment, a fusion protein comprises an HSA moiety that is linked at its C-terminus to the N-terminus of a first linker, which first linker is covalently linked at its C-terminus to the N-terminus of a first FBS moiety, which first FBS moiety is covalently linked at its C-terminus to a second linker, which second linker is covalently linked at its C-terminus to the N-terminus of a second FBS moiety. In another embodiment, a fusion protein comprises a first FBS protein that is covalently linked at its C-terminus to the N-terminus of a first linker, which first linker is covalently linked at its C-terminus to the N-terminus of an HSA moiety, which HSA moiety is covalently linked at its C-terminus to the N-terminus of a second linker, which second linker is covalently linked at its C-terminus to the N-terminus of a second FBS moiety. In each of these constructs, the first and/or the second linker may be absent. These constructs may further comprise one or more FBS moieties. As further described herein, if a fusion protein comprises two or more FBS moieties, the moieties may be binding to the same or to different epitopes of the same or different antigens or targets.

In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; and (ii) an HSA moiety comprising an amino acid sequence that is at least about 70%, 75%, 80%, 85%, 90%, 95%, 97%, 98%, or 99% identical to SEQ ID NO: 102, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA moiety improves at least one property of the FBS moiety relative to a protein consisting of the unmodified FBS moiety. In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA moiety comprising an amino acid sequence that is at least about 70%, 75%, 80%, 85%, 90%, 95%, 97%, 98%, or 99% identical to SEQ ID NO: 102; and (iii) a linker that covalently links the FBS moiety to the HSA moiety, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA moiety improves at least one property of the FBS moiety relative to a protein consisting of the unmodified FBS moiety. In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16, (ii) an HSA moiety comprising an amino acid sequence that is at least about 70%, 75%, 80%, 85%, 90%, 95%, 97%, 98%, or 99% identical to SEQ ID NO: 102; and (iii) a linker that covalently links the FBS moiety to the HSA moiety, wherein the linker comprises 1-10 amino acids, such as 6 amino acids, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA moiety improves at least one property of the FBS moiety relative to a protein consisting of the unmodified FBS moiety. The linker may be a (GS) linker, e.g., (GS)₃ (i.e., GSGSGS (SEQ ID NO: 197)). In a particular embodiment, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA moiety comprising an amino acid sequence that is at least about 70%, 75%, 80%, 85%, 90%, 95%, 97%, 98%, or 99% identical to any one of SEQ ID NO: 102; and (iii) a linker that covalently links the FBS moiety to the HSA moiety, wherein the linker comprises or consists of a (GS)₃ linker (GSGSGS (SEQ ID NO: 197), wherein the HSA moiety improves at least one property of the FBS moiety relative to a protein consisting of the unmodified FBS moiety, and wherein the fusion protein binds specifically to a target (e.g., with a K_{d} of less than 500 nM, 100 nM, 50 nM, 10 nM, 5 nM, 1 nM, 500 pM, 100 pM or less).

In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; and (ii) an HSA moiety comprising the amino acid sequence set forth in SEQ ID NO: 102, wherein the FBS binds specifically to a target (e.g., with a K_{d} of less than 500 nM, 100 nM, 50 nM, 10 nM, 5 nM, 1 nM, 500 pM, 100 pM or less). In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA moiety comprising the amino acid sequence set forth in SEQ ID NO: 102, and (iii) a (GS)₃ linker (e.g., GSGSGS (SEQ ID NO: 197)) that is covalently linked to the FBS moiety and to the HSA moiety, wherein the fusion protein binds specifically to a target (e.g., with a K_{d} of less than 500 nM, 100 nM, 50 nM, 10 nM, 5 nM, 1 nM, 500 pM, 100 pM or less).

Examples of fusion proteins comprising an FBS moiety and an HSA moiety are described in the Examples and have an amino acid sequence set forth as SEQ ID NOs: 198-205.

As set forth herein, linking an HSA moiety to an FBS protein provides specific biological advantages to the FBS protein. Advantages include improved pharmacokinetics (e.g., PK), increased shelf-life, increased solubility, increased affinity for the target and/or increased biological activity of the FBS protein. In certain embodiments, a fusion protein comprising an FBS moiety and an HSA moiety HSA a serum half-life (e.g., in mice, Cynomolgus monkeys or humans) that is at least 2, 5, 10, 20, 30, 40, 50, 60, 70, 80, 90, 100 or more longer than the half life of the FBS moiety that is not linked to an HSA moiety (e.g., relative to the unmodified FBS moiety). For example, the serum (or plasma) half-life (e.g., in a mouse, Cynomolgus monkey or human) may be at least 1 day, 2 days, 3 days, 4 days, 5 days, 6 days, 7 days, 8 days, 9 filings or 10 days or longer than that of the unmodified FBS moiety. The half-life may also be indicated in hours and may be, (e.g., in mice, Cynomolgus monkey, or humans) at least 25 hours, 50 hours, 75 hours, 100 hours, 150 hours, 200 hours or more.

In certain embodiments, a fusion protein comprising an FBS moiety and an HSA moiety HSA a shelf half-life that is at least 2, 3, 4, 5, 6, 7, 8, 9, 10, 20 or more times longer than that of the unmodified FBS. For example, a fusion protein may have a shelf-life of at least 1, 2, 3, 4, 5, 6, 7, 8, 9, 10, 11, 12 or more months.

In certain embodiments, a fusion protein comprising an FBS moiety and an HSA moiety is present in a composition, e.g., a pharmaceutical composition, at a concentration of at least 10 mg/ml, 20 mg/ml, 30 mg/ml, 40 mg/ml, 50 mg/ml, 60 mg/ml, 70 mg/ml, 80 mg/ml, 90 mg/ml or 100 mg/ml.

In certain embodiments, a fusion protein comprising an FBS moiety and an HSA moiety is present in a composition, e.g., a pharmaceutical composition, mostly as a monomer, e.g., at least 80%, 85%, 90%, 95%, 98%, or 99% of the fusion protein in the composition is in monomeric form.

In certain embodiments, a fusion protein comprising an FBS moiety and an HSA moiety HSA biological activity that is as least as strong as that of the unmodified FBS moiety. Biological activity can be binding affinity to a target or a biological activity in an assay, e.g., the ability to destroy tumor cells. In certain embodiments, the biological activity of the fusion protein is stronger (e.g., by 5%, 10%, 25%, 50%, 100%, 2 fold nor more) than that of the unmodified FBS moiety.

A fusion protein comprising an FBS and an HSA moiety may also comprise a combination of the above characteristics. For example, a fusion protein may have a serum half-life (in Cynomolgus monkeys or humans) of at least 50 hours, have a shelf-life of at least one month, be soluble at concentrations of up to 50 mg/ml, be present at least 90% in monomeric form, and/or have a biological activity that is at least as potent as that of the unmodified FBS.

### Exemplary Fusion Proteins Comprising an FBS Moiety and an HSA Binding Moiety

Provided herein are fusion proteins (e.g., target binding fusion proteins) comprising an amino acid sequence encoding an FBS moiety that binds specifically to a given target (e.g., a target protein) and an amino acid sequence that encodes an HSA binding moiety. The FBS moiety may be located C-terminally or N-terminally to the HSA binding moiety. The FBS moiety may be linked directly (i.e., without one or more intervening amino acids) or indirectly to the HSA binding moiety. An “indirect linkage” between two protein moieties refers to a linkage between two protein moieties that comprises at least one intervening amino acid. In one embodiment, a fusion protein comprises an FBS moiety that is covalently linked at its C-terminus to the N-terminus of a linker, which linker is covalently linked at its C-terminus to the N-terminus of an HSA binding moiety. In another embodiment, a fusion protein comprises an HSA binding moiety that is covalently linked at its C-terminus to the N-terminus of a linker, which linker is covalently linked at its C-terminus to the N-terminus of an FBS moiety. In another embodiment, a fusion protein comprises a first FBS moiety that is covalently linked at its C-terminus to the N-terminus of a second FBS moiety, which second FBS moiety is covalently linked at its C-terminus to the N-terminus of a linker, which linker is covalently linked at its C-terminus to the N-terminus of an HSA binding moiety. In another embodiment, a fusion protein comprises a first FBS moiety that is covalently linked at its C-terminus to the N-terminus of a first linker, which first linker is covalently linked at its C-terminus to the N-terminus of a second FBS moiety, which second FBS moiety is covalently linked at its C-terminus to the N-terminus of a second linker, which second linker is covalently linked at its C-terminus to the N-terminus of an HSA binding moiety. In another embodiment, a fusion protein comprises an HSA binding moiety that is linked at its C-terminus to the N-terminus of a first linker, which first linker is covalently linked at its C-terminus to the N-terminus of a first FBS moiety, which first FBS moiety is covalently linked at its C-terminus to a second linker, which second linker is covalently linked at its C-terminus to the N-terminus of a second FBS moiety. In another embodiment, a fusion protein comprises a first FBS protein that is covalently linked at its C-terminus to the N-terminus of a first linker, which first linker is covalently linked at its C-terminus to the N-terminus of an HSA binding moiety, which HSA binding moiety is covalently linked at its C-terminus to the N-terminus of a second linker, which second linker is covalently linked at its C-terminus to the N-terminus of a second FBS moiety. In each of these constructs, the first and/or the second linker may be absent. These constructs may further comprise one or more FBS moieties. As further described herein, if a fusion protein comprises two or more FBS moieties, the moieties may be binding to the same or to different epitopes of the same or different antigens or targets.

In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; and (ii) an HSA binding moiety comprising an amino acid sequence that is at least about 70%, 75%, 80%, 85%, 90%, 95%, 97%, 98%, or 99% identical to any one of SEQ ID NOs: 103-112, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA binding moiety improves at least one property of the FBS moiety relative to a protein consisting of the unmodified FBS moiety. In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA binding moiety comprising an amino acid sequence that is at least about 70%, 75%, 80%, 85%, 90%, 95%, 97%, 98%, or 99% identical to any one of SEQ ID NOs: 103-112; and (iii) a linker that covalently links the FBS moiety to the HSA binding moiety, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA binding moiety improves at least one property of the FBS moiety relative to a protein consisting of the unmodified FBS moiety. In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16, (ii) an HSA binding moiety comprising an amino acid sequence that is at least about 70%, 75%, 80%, 85%, 90%, 95%, 97%, 98%, or 99% identical to any one of SEQ ID NOs: 103-112; and (iii) a linker that covalently links the FBS moiety to the HSA binding moiety, wherein the linker comprises 1-10 amino acids, such as 6 amino acids, wherein the fusion protein binds specifically to a target (e.g., with a K_{d} of less than 500 nM, 100 nM, 50 nM, 10 nM, 5 nM, 1 nM, 500 pM, 100 pM or less) and wherein the HSA binding moiety improves at least one property of the FBS moiety relative to a protein consisting of the unmodified FBS moiety. The linker may be a (GS) linker, e.g., (GS)₃ (i.e., GSGSGS (SEQ ID NO: 197)). In a particular embodiment, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA binding moiety comprising an amino acid sequence that is at least about 70%, 75%, 80%, 85%, 90%, 95%, 97%, 98%, or 99% identical to any one of SEQ ID NOs: 103-112; and (iii) a linker that covalently links the FBS moiety to the HSA binding moiety, wherein the linker comprises, consists essentially of, or consists of a (GS)₃ linker (GSGSGS (SEQ ID NO: 197)), wherein the HSA binding moiety improves at least one property of the FBS moiety relative to a protein consisting of the unmodified FBS moiety, and wherein the fusion protein binds specifically to a target (e.g., with a Kₐ of less than 500 nM, 100 nM, 50 nM, 10 nM, 5 nM, 1 nM, 500 pM, 100 pM or less).

In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; and (ii) an HSA binding moiety comprising an amino acid sequence selected from the group of sequences consisting of SEQ ID NOs: 103-153, wherein the FBS binds specifically to a target (e.g., with a K_{d} of less than 500 nM, 100 nM, 50 nM, 10 nM, 5 nM, 1 nM, 500 pM, 100 pM or less). In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA binding moiety comprising the amino acid sequence an HSA binding moiety comprising an amino acid sequence selected from the group of sequences consisting of SEQ ID NOs: 103-153, and (iii) a (GS)₃ linker (e.g., GSGSGS (SEQ ID NO: 197)) that is covalently linked to the FBS moiety and to the HSA binding moiety, wherein the fusion protein binds specifically to a target (e.g., with a K_{d} of less than 500 nM, 100 nM, 50 nM, 10 nM, 5 nM, 1 nM, 500 pM, 100 pM or less).

In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; and (ii) an HSA binding moiety comprising the amino acid sequence set forth as any one of SEQ ID NOs: 103-112, wherein the FBS binds specifically to a target (e.g., with a K_{d} of less than 500 nM, 100 nM, 50 nM, 10 nM, 5 nM, 1 nM, 500 pM, 100 pM or less). In certain embodiments, a fusion protein comprises (i) an FBS moiety comprising an amino acid sequence that is at least about 70%, 75%, 80%, 85%, 90%, 95%, 97%, 98%, or 99% identical to any one of SEQ ID NOs: 1-16; (ii) an HSA binding moiety comprising the amino acid sequence set forth as any one of SEQ ID NOs: 103-112, and (iii) a (GS)₃ linker (e.g., GSGSGS (SEQ ID NO: 197)) that is covalently linked to the FBS moiety and to the HSA binding moiety, wherein the fusion protein binds specifically to a target (e.g., with a K_{d} of less than 500 nM, 100 nM, 50 nM, 10 nM, 5 nM, 1 nM, 500 pM, 100 pM or less).

An exemplary fusion protein comprising an FBS moiety and an HSA binding moiety is described in the Examples and has an amino acid sequence set forth as SEQ ID NO: 154. An ABD domain may also be linked to any of the other FBS moieties described herein.

As set forth herein, linking an HSA binding moiety to an FBS protein provides specific biological advantages to the FBS protein. Advantages include improved pharmacokinetics (e.g., PK), increased shelf-life, increased solubility, increased affinity for the target and/or increased biological activity of the FBS protein. In certain embodiments, a fusion protein comprising an FBS moiety and an HSA binding moiety HSA a serum half-life (e.g., in mice, Cynomolgus monkeys or humans) that is at least 2, 5, 10, 20, 30, 40, 50, 60, 70, 80, 90, 100 or more longer than the half life of the FBS moiety that is not linked to an HSA binding moiety (e.g., relative to the unmodified FBS moiety). For example, the serum (or plasma) half-life (e.g., in a mouse, Cynomolgus monkey or human) may be at least 1 day, 2 days, 3 days, 4 days, 5 days, 6 days, 7 days, 8 days, 9 filings or 10 days or longer than that of the unmodified FBS moiety. The half-life may also be indicated in hours and may be, (e.g., in mice, Cynomolgus monkey, or humans) at least 25 hours, 50 hours, 75 hours, 100 hours, 150 hours, 200 hours or more.

In certain embodiments, a fusion protein comprising an FBS moiety and an HSA binding moiety has a shelf half-life that is at least 2, 3, 4, 5, 6, 7, 8, 9, 10, 20 or more times longer than that of the unmodified FBS. For example, a fusion protein may have a shelf-life of at least 1, 2, 3, 4, 5, 6, 7, 8, 9, 10, 11, 12 or more months.

In certain embodiments, a fusion protein comprising an FBS moiety and an HSA binding moiety is present in a composition, e.g., a pharmaceutical composition, at a concentration of at least 10 mg/ml, 20 mg/ml, 30 mg/ml, 40 mg/ml, 50 mg/ml, 60 mg/ml, 70 mg/ml, 80 mg/ml, 90 mg/ml or 100 mg/ml.

In certain embodiments, a fusion protein comprising an FBS moiety and an HSA binding moiety is present in a composition, e.g., a pharmaceutical composition, mostly as a monomer, e.g., at least 80%, 85%, 90%, 95%, 98%, or 99% of the fusion protein in the composition is in monomeric form.

In certain embodiments, a fusion protein comprising an FBS moiety and an HSA binding moiety HSA biological activity that is as least as strong as that of the unmodified FBS moiety. Biological activity can be binding affinity to a target or a biological activity in an assay, e.g., the ability to destroy tumor cells. In certain embodiments, the biological activity of the fusion protein is stronger (e.g., by 5%, 10%, 25%, 50%, 100%, 2 fold nor more) than that of the unmodified FBS moiety.

A fusion protein comprising an FBS and an HSA binding moiety may also comprise a combination of the above characteristics. For example, a fusion protein may have a serum half-life (in Cynomolgus monkeys or humans) of at least 50 hours, have a shelf-life of at least one month, be soluble at concentrations of up to 50 mg/ml, be present at least 90% in monomeric form, and/or have a biological activity that is at least as potent as that of the unmodified FBS.

### Vectors and Polynucleotides Embodiments

Nucleic acids encoding any of the various fusion proteins comprising an FBS moiety and an HSA or HSA binding moiety disclosed herein may be synthesized chemically, enzymatically or recombinantly. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100(2):438-442 (Jan. 21, 2003); Sinclair et al., Protein Expr. Purif., 26(1):96-105 (October 2002); Connell, N. D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described for example in Sambrook et al., Molecular Cloning: A Laboratory Manual, Second Edition, Vols. 1-3, Cold Spring Harbor Laboratory Press (1989), or Ausubel, F. et al., Current Protocols in Molecular Biology, Green Publishing and Wiley-Interscience: New York (1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated.

The fusion proteins described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces alpha-factor leaders), or acid phosphatase leader, the C. albicans glucoamylase leader, or the signal described in PCT Publication No. WO 90/13646. In mammalian cell expression, mammalian signal sequences as well as viral secretory leaders, for example, the herpes simplex gD signal, are available. The DNA for such precursor regions may be ligated in reading frame to DNA encoding the protein. In embodiments in which the HSA moiety is located N-terminally to other moieties in a fusion protein, the signal sequence used may be that of the naturally occurring HSA signal sequence, e.g., set forth as amino acids 1-24 of SEQ ID NO: 101.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC® No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC® 20,622 or 38,626) are complemented by known plasmids bearing the Leu2 gene.

Expression and cloning vectors usually contain a promoter that is recognized by the host organism and is operably linked to the nucleic acid encoding the fusion protein. Promoters suitable for use with prokaryotic hosts include the phoA promoter, beta-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the fusion protein.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT (SEQ ID NO: 206) region where N may be any nucleotide. At the 3′ end of most eukaryotic genes is an AATAAA (SEQ ID NO: 207) sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP Patent Publication No. 73,657 and PCT Publication Nos. WO 2011/124718 and WO 2012/059486. Yeast enhancers also are advantageously used with yeast promoters.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the Actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature, 297:598-601 (1982) on expression of human β-interferon cDNA in mouse cells under the control of a thymidine kinase promoter from herpes simplex virus. Alternatively, the rous sarcoma virus long terminal repeat can be used as the promoter.

Transcription of a DNA encoding a fusion protein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the polypeptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide. One useful transcription termination component is the bovine growth hormone polyadenylation region. See WO 94/11026 and the expression vector disclosed therein.

The recombinant DNA can also include any type of protein tag sequence that may be useful for purifying the fusion proteins. Examples of protein tags include but are not limited to a histidine tag, a FLAG® tag, a myc tag, an HA tag, or a GST tag. Appropriate cloning and expression vectors for use with bacterial, fungal, yeast, and mammalian cellular hosts can be found in Cloning Vectors: A Laboratory Manual, Elsevier, New York (1985), the relevant disclosure of which is hereby incorporated by reference.

The expression construct may be introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified fusion proteins are prepared by culturing suitable host/vector systems to express the recombinant proteins. The FBS protein is then purified from culture media or cell extracts.

### Protein Production

Host cells are transformed with the herein-described expression or cloning vectors for protein production and cultured in conventional nutrient media modified as appropriate for inducing promoters, selecting transformants, or amplifying the genes encoding the desired sequences.

The host cells used to produce the fusion proteins may be cultured in a variety of media. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enzymol., 58:44 (1979), Barnes et al., Anal. Biochem., 102:255 (1980), U.S. Pat. Nos. 4,767,704; 4,657,866; 4,927,762; 4,560,655; or 5,122,469; PCT Publication Nos. WO 90/03430; WO 87/00195; or U.S. Pat. No. RE30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Fusion proteins disclosed herein can also be produced using cell-free translation systems. For such purposes the nucleic acids encoding the fusion protein must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Fusion proteins can also be produced by chemical synthesis (e.g., by the methods described in Solid Phase Peptide Synthesis, Second Edition, The Pierce Chemical Co., Rockford, Ill. (1984)). Modifications to the fusion protein can also be produced by chemical synthesis.

The fusion proteins disclosed herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, fusion proteins may be exchanged into different buffers and/or concentrated by any of a variety of methods known to the art, including, but not limited to, filtration and dialysis.

The purified fusion protein is preferably at least 85% pure, more preferably at least 95% pure, and most preferably at least 98% or 99% pure. Regardless of the exact numerical value of the purity, the fusion protein is sufficiently pure for use as a pharmaceutical product.

### Exemplary Uses

In one aspect, the application provides fusion proteins comprising FBS moieties useful in the treatment of disorders. The diseases or disorders that may be treated will be dictated by the binding specificity of the FBS moiety. As described herein, FBS moieties may be designed to bind to any target of interest. Exemplary targets include, for example, TNF-alpha, VEGFR2, PCSK9, IL-23, EGFR and IGF1R. Merely as an example, FBS moieties that bind to TNF-alpha may be used to treat autoimmune disorders such as rheumatoid arthritis, inflammatory bowel disease, psoriasis, and asthma. Fusion proteins described herein may also be used for treating cancer.

The application also provides methods for administering fusion proteins to a subject. In some embodiments, the subject is a human. In some embodiments, the fusion proteins are pharmaceutically acceptable to a mammal, in particular a human. A “pharmaceutically acceptable” composition refers to a composition that is administered to an animal without significant adverse medical consequences. Examples of pharmaceutically acceptable compositions include compositions comprising fibronecting based scaffold moieties that lack the integrin-binding domain (RGD) and compositions that are essentially endotoxin or pyrogen free or have very low endotoxin or pyrogen levels.

### Formulation and Administration

The application further provides pharmaceutically acceptable compositions comprising the fusion proteins described herein, wherein the composition is essentially endotoxin and/or pyrogen free.

Therapeutic formulations comprising fusion proteins are prepared for storage by mixing the described proteins having the desired degree of purity with optional physiologically acceptable carriers, excipients or stabilizers (Osol, A., ed., Remington's Pharmaceutical Sciences, 16th Edition (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, PLURONIC® or polyethylene glycol (PEG).

The formulations herein may also contain more than one active compounds as necessary for the particular indication being treated, preferably those with complementary activities that do not adversely affect each other. Such molecules are suitably present in combination in amounts that are effective for the purpose intended.

The fusion proteins may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Osol, A., ed., Remington's Pharmaceutical Sciences, 16th Edition (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the fusion proteins described herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

While the skilled artisan will understand that the dosage of each fusion protein will be dependent on the identity of the protein, the preferred dosages can range from about 10 mg/square meter to about 2000 mg/square meter, more preferably from about 50 mg/square meter to about 1000 mg/square meter.

For therapeutic applications, the fusion proteins are administered to a subject, in a pharmaceutically acceptable dosage form. They can be administered intravenously as a bolus or by continuous infusion over a period of time, by intramuscular, subcutaneous, intra-articular, intrasynovial, intrathecal, oral, topical, or inhalation routes. The protein may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose. The methods of the present invention can be practiced in vitro, in vivo, or ex vivo.

Administration of fusion proteins, and one or more additional therapeutic agents, whether co-administered or administered sequentially, may occur as described above for therapeutic applications. Suitable pharmaceutically acceptable carriers, diluents, and excipients for co-administration will be understood by the skilled artisan to depend on the identity of the particular therapeutic agent being co-administered.

When present in an aqueous dosage form, rather than being lyophilized, the fusion protein typically will be formulated at a concentration of about 0.1 mg/ml to 100 mg/ml, although wide variation outside of these ranges is permitted. For the treatment of disease, the appropriate dosage of fusion proteins will depend on the type of disease to be treated, the severity and course of the disease, whether the fusion proteins are administered for preventive or therapeutic purposes, the course of previous therapy, the patient's clinical history and response to the fusion, and the discretion of the attending physician. The fusion protein is suitably administered to the patient at one time or over a series of treatments.

### SEQUENCES

Wild-type human ¹⁰Fn3 Domain version 1:

(SEQ ID NO: 1)

VSDVPRDLEVVAATPTSLLISWDAPAVTVRYYRITYGETGGNSPVQEFTV
PGSKSTATISGLKPGVDYTITVYAVTGRGDSPASSKPISINYRT
Wild-type human ¹⁰Fn3 Domain version 2:

(SEQ ID NO: 2)

SDVPRDLEVVAATPTSLLISWDAPAVTVRYYRITYGETGGNSPVQEFTVP
GSKSTATISGLKPGVDYTITVYAVTGRGDSPASSKPISINYRT
Wild-type human ¹⁰Fn3 Domain version 3:

(SEQ ID NO: 3)

DVPRDLEVVAATPTSLLISWDAPAVTVRYYRITYGETGGNSPVQEFTVPG
SKSTATISGLKPGVDYTITVYAVTGRGDSPASSKPISINYRT
Wild-type human ¹⁰Fn3 Domain version 4:

(SEQ ID NO: 4)

VPRDLEVVAATPTSLLISWDAPAVTVRYYRITYGETGGNSPVQEFTVPGS
KSTATISGLKPGVDYTITVYAVTGRGDSPASSKPISINYRT
Wild-type human ¹⁰Fn3 Domain version 5:

(SEQ ID NO: 5)

PRDLEVVAATPTSLLISWDAPAVTVRYYRITYGETGGNSPVQEFTVPGSK
STATISGLKPGVDYTITVYAVTGRGDSPASSKPISINYRT
Wild-type human ¹⁰Fn3 Domain version 6:

(SEQ ID NO: 6)

RDLEVVAATPTSLLISWDAPAVTVRYYRITYGETGGNSPVQEFTVPGSKS
TATISGLKPGVDYTITVYAVTGRGDSPASSKPISINYRT
Wild-type human ¹⁰Fn3 Domain version 7:

(SEQ ID NO: 7)

DLEVVAATPTSLLISWDAPAVTVRYYRITYGETGGNSPVQEFTVPGSKST
ATISGLKPGVDYTITVYAVTGRGDSPASSKPISINYRT
Wild-type human ¹⁰Fn3 Domain version 8:

(SEQ ID NO: 8)

LEVVAATPTSLLISWDAPAVTVRYYRITYGETGGNSPVQEFTVPGSKSTA
TISGLKPGVDYTITVYAVTGRGDSPASSKPISINYRT
Wild-type human ¹⁰Fn3 Domain version 1 with
N-terminal tail:

(SEQ ID NO: 9)

VSDVPRDLEVVAATPTSLLISWDAPAVTVRYYRITYGETGGNSPVQEFTV
PGSKSTATISGLKPGVDYTITVYAVTGRGDSPASSKPISINYRTEIDKPS
Q
Human ¹⁰Fn3 Domain version 1 with
N-terminal tail having D97E:

(SEQ ID NO: 10)

VSDVPRDLEVVAATPTSLLISWDAPAVTVRYYRITYGETGGNSPVQEFTV
PGSKSTATISGLKPGVDYTITVYAVTGRGDSPASSKPISINYRTEIEKPS
Q
Wild-type human ¹⁰Fn3 Domain version 2 with
N-terminal tail:

(SEQ ID NO: 11)

SDVPRDLEVVAATPTSLLISWDAPAVTVRYYRITYGETGGNSPVQEFTVP
GSKSTATISGLKPGVDYTITVYAVTGRGDSPASSKPISINYRTEIDKPSQ
Human ¹⁰Fn3 Domain version 2 with
N-terminal tail having D97E:

(SEQ ID NO: 12)

SDVPRDLEVVAATPTSLLISWDAPAVTVRYYRITYGETGGNSPVQEFTVP
GSKSTATISGLKPGVDYTITVYAVTGRGDSPASSKPISINYRTEIEKPSQ
Wild-type human ¹⁰Fn3 Domain version 3 with
N-terminal tail:

(SEQ ID NO: 13)

DVPRDLEVVAATPTSLLISWDAPAVTVRYYRITYGETGGNSPVQEFTVPG
SKSTATISGLKPGVDYTITVYAVTGRGDSPASSKPISINYRTEIDKPSQ
Human ¹⁰Fn3 Domain version 3 with
N-terminal tail having D97E:

(SEQ ID NO: 14)

DVPRDLEVVAATPTSLLISWDAPAVTVRYYRITYGETGGNSPVQEFTVPG
SKSTATISGLKPGVDYTITVYAVTGRGDSPASSKPISINYRTEIEKPSQ
Wild-type human ¹⁰Fn3 Domain version 4 with
N-terminal tail:

(SEQ ID NO: 15)

VPRDLEVVAATPTSLLISWDAPAVTVRYYRITYGETGGNSPVQEFTVPGS
KSTATISGLKPGVDYTITVYAVTGRGDSPASSKPISINYRTEIDKPSQ
Human ¹⁰Fn3 Domain version 4 with
N-terminal tail having D97E:

(SEQ ID NO: 16)

VPRDLEVVAATPTSLLISWDAPAVTVRYYRITYGETGGNSPVQEFTVPGS
KSTATISGLKPGVDYTITVYAVTGRGDSPASSKPISINYRTEIEKPSQ
Wild-type human ¹⁰Fn3 Domain version 5 with
N-terminal tail:

(SEQ ID NO: 17)

PRDLEVVAATPTSLLISWDAPAVTVRYYRITYGETGGNSPVQEFTVPGSK
STATISGLKPGVDYTITVYAVTGRGDSPASSKPISINYRTEIDKPSQ
Human ¹⁰Fn3 Domain version 5 with
N-terminal tail having D97E:

(SEQ ID NO: 18)

PRDLEVVAATPTSLLISWDAPAVTVRYYRITYGETGGNSPVQEFTVPGSK
STATISGLKPGVDYTITVYAVTGRGDSPASSKPISINYRTEIEKPSQ
Wild-type human ¹⁰Fn3 Domain version 6 with
N-terminal tail:

(SEQ ID NO: 19)

RDLEVVAATPTSLLISWDAPAVTVRYYRITYGETGGNSPVQEFTVPGSKS
TATISGLKPGVDYTITVYAVTGRGDSPASSKPISINYRTEIDKPSQ
Human ¹⁰Fn3 Domain version 6 with
N-terminal tail having D97E:

(SEQ ID NO: 20)

RDLEVVAATPTSLLISWDAPAVTVRYYRITYGETGGNSPVQEFTVPGSKS
TATISGLKPGVDYTITVYAVTGRGDSPASSKPISINYRTEIEKPSQ
Wild-type human ¹⁰Fn3 Domain version 7 with
N-terminal tail:

(SEQ ID NO: 21)

DLEVVAATPTSLLISWDAPAVTVRYYRITYGETGGNSPVQEFTVPGSKST
ATISGLKPGVDYTITVYAVTGRGDSPASSKPISINYRTEIDKPSQ
Human ¹⁰Fn3 Domain version 7 with
N-terminal tail having D97E:

(SEQ ID NO: 22)

DLEVVAATPTSLLISWDAPAVTVRYYRITYGETGGNSPVQEFTVPGSKST
ATISGLKPGVDYTITVYAVTGRGDSPASSKPISINYRTEIEKPSQ
Wild-type human ¹⁰Fn3 Domain version 8 with
N-terminal tail:

(SEQ ID NO: 23)

LEVVAATPTSLLISWDAPAVTVRYYRITYGETGGNSPVQEFTVPGSKSTA
TISGLKPGVDYTITVYAVTGRGDSPASSKPISINYRTEIDKPSQ
Human ¹⁰Fn3 Domain version 8 with
N-terminal tail having D97E:

(SEQ ID NO: 24)

LEVVAATPTSLLISWDAPAVTVRYYRITYGETGGNSPVQEFTVPGSKSTA
TISGLKPGVDYTITVYAVTGRGDSPASSKPISINYRTEIEKPSQ
Degenerate wild-type human ¹⁰Fn3 Domain
version 1:

(SEQ ID NO: 25)

VSDVPRDLEVVAA(X)ᵤLLISW(X)ᵥYRITY(X)_{w}
FTV(X)ₓATISGL(X)_{y}YTITVYA(X)_{z}ISINYRT
Degenerate wild-type human ¹⁰Fn3 Domain
version 2:

(SEQ ID NO: 26)

SDVPRDLEVVAA(X)ᵤLLISW(X)ᵥYRITY(X)_{w}
FTV(X)ₓATISGL(X)_{y}YTITVYA(X)_{z}ISINYRT
Degenerate wild-type human ¹⁰Fn3 Domain
version 3:

(SEQ ID NO: 27)

DVPRDLEVVAA(X)ᵤLLISW(X)ᵥYRITY(X)_{w}
FTV(X)ₓATISGL(X)_{y}YTITVYA(X)_{z}ISINYRT
Degenerate wild-type human ¹⁰Fn3 Domain
version 4:

(SEQ ID NO: 28)

VPRDLEVVAA(X)ᵤLLISW(X)ᵥYRITY(X)_{w}
FTV(X)ₓATISGL(X)_{y}YTITVYA(X)_{z}ISINYRT
Degenerate wild-type human ¹⁰Fn3 Domain
version 5:

(SEQ ID NO: 29)

PRDLEVVAA(X)ᵤLLISW(X)ᵥYRITY(X)_{w}
FTV(X)ₓATISGL(X)_{y}YTITVYA(X)_{z}ISINYRT
Degenerate wild-type human ¹⁰Fn3 Domain
version 6:

(SEQ ID NO: 30)

RDLEVVAA(X)ᵤLLISW(X)ᵥYRITY(X)_{w}
FTV(X)ₓATISGL(X)_{y}YTITVYA(X)_{z}
ISINYRT
Degenerate wild-type human ¹⁰Fn3 Domain
version 7:

(SEQ ID NO: 31)

DLEVVAA(X)ᵤLLISW(X)ᵥYRITY(X)_{w}
FTV(X)ₓATISGL(X)_{y}YTITVYA(X)_{z}I
SINYRT
Degenerate wild-type human ¹⁰Fn3 Domain
version 8:

(SEQ ID NO: 32)

LEVVAA(X)ᵤLLISW(X)ᵥYRITY(X)_{w}F
TV(X)ₓATISGL(X)_{y}YTITVYA(X)_{z}IS
INYRT
Degenerate wild-type human ¹⁰Fn3 Domain
version 1 with N-terminal tail:

(SEQ ID NO: 33)

VSDVPRDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 1 with N-terminal tail having D97E:

(SEQ ID NO: 34)

VSDVPRDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 2 with N-terminal tail:

(SEQ ID NO: 35)

SDVPRDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 2 with N-terminal tail having D97E:

(SEQ ID NO: 36)

SDVPRDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 3 with N-terminal tail:

(SEQ ID NO: 37)

DVPRDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 3 with N-terminal tail having D97E:

(SEQ ID NO: 38)

DVPRDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 4 with N-terminal tail:

(SEQ ID NO: 39)

VPRDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 4 with N-terminal tail having D97E:

(SEQ ID NO: 40)

VPRDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 5 with N-terminal tail:

(SEQ ID NO: 41)

PRDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 5 with N-terminal tail having D97E:

(SEQ ID NO: 42)

PRDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 6 with N-terminal tail:

(SEQ ID NO: 43)

RDLEVVAA(X)ᵤLLISW(X)ᵥYRITY(X)_{w}
FTV(X)ₓATISGL(X)_{y}YTITVYA(X)_{z}
ISINYRTEIDKPSQ
Degenerate wild-type human ¹⁰Fn3 Domain
version 6 with N-terminal tail having D97E:

(SEQ ID NO: 44)

RDLEVVAA(X)ᵤLLISW(X)ᵥYRITY(X)_{w}
FTV(X)ₓATISGL(X)_{y}YTITVYA(X)_{z}
ISINYRTEIEKPSQ
Degenerate wild-type human ¹⁰Fn3 Domain
version 7 with N-terminal tail:

(SEQ ID NO: 45)

DLEVVAA(X)ᵤLLISW(X)ᵥYRITY(X)_{w}
FTV(X)ₓATISGL(X)_{y}YTITVYA(X)_{z}I
SINYRTEIDKPSQ
Degenerate wild-type human ¹⁰Fn3 Domain
version 7 with N-terminal tail having D97E:

(SEQ ID NO: 46)

DLEVVAA(X)ᵤLLISW(X)ᵥYRITY(X)_{w}
FTV(X)ₓATISGL(X)_{y}YTITVYA(X)_{z}I
SINYRTEIEKPSQ
Degenerate wild-type human ¹⁰Fn3 Domain
version 8 with N-terminal tail:

(SEQ ID NO: 47)

LEVVAA(X)ᵤLLISW(X)ᵥYRITY(X)_{w}F
TV(X)ₓATISGL(X)_{y}YTITVYA(X)_{z}IS
INYRTEIDKPSQ
Degenerate wild-type human ¹⁰Fn3 Domain
version 8 with N-terminal tail having D97E:

(SEQ ID NO: 48)

LEVVAA(X)ᵤLLISW(X)ᵥYRITY(X)_{w}F
TV(X)ₓATISGL(X)_{y}YTITVYA(X)_{z}IS
INYRTEIEKPSQ
Wild-type human ¹⁰Fn3 Core Domain:

(SEQ ID NO: 49)

LEVVAATPTSLLISWDAPAVTVRYYRITYGETGGNSPVQEFTVPGSKSTA
TISGLKPGVDYTITVYAVTGRGDSPASSKPISINYRT
Degenerate wild-type human ¹⁰Fn3 Core
Domain:

(SEQ ID NO: 50)

LEVVAA(X)ᵤLLISW(X)ᵥYRITY(X)_{w}F
TV(X)ₓATISGL(X)_{y}YTITVYA(X)_{z}IS
INYRT
Wild-type human ¹⁰Fn3 N-terminal sequence
version 1:

(SEQ ID NO: 51)

VSDVPRD
Wild-type human ¹⁰Fn3 N-terminal sequence
version 2:

(SEQ ID NO: 52)

SDVPRD
Wild-type human ¹⁰Fn3 N-terminal sequence
version 3:

(SEQ ID NO: 53)

DVPRD
Wild-type human ¹⁰Fn3 N-terminal sequence
version 4:

(SEQ ID NO: 54)

VPRD
Wild-type human ¹⁰Fn3 tail:

(SEQ ID NO: 55)

EIDKPSQ
Human ¹⁰Fn3 tail having D97E:

(SEQ ID NO: 56)

EIEKPSQ

(SEQ ID NO: 57)

MGVSDVPRDL

(SEQ ID NO: 58)

GVSDVPRDL

(SEQ ID NO: 59)

XₙSDVPRDL

(SEQ ID NO: 60)

XₙDVPRDL

(SEQ ID NO: 61)

XₙVPRDL

(SEQ ID NO: 62)

XₙPRDL

(SEQ ID NO: 63)

XₙRDL

(SEQ ID NO: 64)

XₙDL

(SEQ ID NO: 65)

MASTSG

(SEQ ID NO: 66)

EIEK

(SEQ ID NO: 67)

EGSGC

(SEQ ID NO: 68)

EIEKPCQ

(SEQ ID NO: 69)

EIEKP

(SEQ ID NO: 70)

EIEKPS

(SEQ ID NO: 71)

EIEKPC

(SEQ ID NO: 72)

HHHHHH

(SEQ ID NO:)73

EIDK

(SEQ ID NO: 74)

EIDKPCQ

The following representative Examples contain important additional information, exemplification and guidance which can be adapted to the practice of this invention in its various embodiments and the equivalents thereof. These examples are intended to help illustrate the invention, and are not intended to, nor should they be construed to, limit its scope.

### EXAMPLES

### Example 1

### Adnectin-HSA Fusion Constructs

Eight constructs encoding FBS-HSA fusion proteins were made for expression in mammalian cells: an IL-23 binding adnectin linked at its C-terminus to HSA; an IL-23 binding adnectin linked at its N-terminus to HSA; a PCSK9 binding adnectin linked at its C-terminus to HSA; a PCSK9 binding adnectin linked at its N-terminus to HSA; an IGF1R binding adnectin linked at its C-terminus to an EGFR binding adnectin linked at its C-terminus to HSA; HSA linked at its C-terminus to an IGF1R binding adnectin linked at its C-terminus to an EGFR binding adnectin; an IGF1R binding adnectin linked at its C-terminus to HSA linked at its C-terminus to an EGFR binding adnectin; and an EGFR binding adnectin linked at its C-terminus to HSA linked at its C-terminus to an IGF1R binding adnectin. The amino acid sequence of each of the constructs is shown in FIG. 1. The constructs were expressed in the human cells HEK-292.

### Example 2

### Half-Life Extension of a PCSK9 Adnectin-ABD Fusion

This Example shows that a PCSK9 adnectin linked to ABD has the expected biological activity and has an extended half-life relative to the adnectin that is not linked to ABD.

A construct encoding a fusion protein consisting of a PCSK9 adnectin linked at its C-terminus to a linker, which is linked at its C-terminus to the N-terminus of ABD domain having SEQ ID NO: 112 was prepared. The sequence of the fusion protein encoded by the construct is as follows:

MGVSDVPRDLEVVAATPTSLLISWVPPSDDYGYYRITYGETGGNSPVQEF
TVPIGKGTATISGLKPGVDYTITVYAVEFPWPHAGYYHRPISINYRTGSG
SGSQHDEAVDANSLAEAKVLANRELDKYGVSDYYKNLINNAKTVEGVKAL
IDEILAALPHHHHHH (SEQ ID NO: 208; and consists of
SEQ ID NO: 154 plus the histidine tail).

The fusion protein was expressed in E. coli and purified.

### Assessment of PCSK9 Adnectin-ABD Affinity and Potency in FRET Assays

A fluorescence resonance energy transfer (FRET)-based assay was developed to determine the binding affinity and potency of PCSK9-binding adnectins. It was adapted from the general method described previously by Maio et al. (Miao, B. et al., Meth. Enzymol., 357:180-188 (2002); see also WO 2011/130354). The PCSK9:EGFA FRET assay measures the inhibition of PCSK9 binding to the LDLR epidermal growth factor precursor homology domain (EGFA domain), using recombinant human PCSK9 expressed in baculovirus and a synthetic 40-mer EGFA peptide (biotinylated). EGFA HSA been shown to represent the key interacting domain of LDLR with PCSK9. This assay uses a PCSK9 C-terminal domain binding mAb (mAb 4H5) labeled with Eu-chelate to provide FRET interaction with biotinylated EGFA through the streptavidin/allophycocyanin fluorophore complex. The results indicate that the EC₅₀ of the EGFA FRET assay is 0.30 nM for the PCSK9 adnectin-ABD fusion protein, compared to 2.6±1.2 nM for the unmodified (i.e., without ABD) PCSK9 adnectin (see Table 1). Thus, the biological activity of PCSK9 adnectin linked to ABD is at least as good as that of the unmodified PCSK9 adnectin.

### Assessment of PCSK9 Adnectin-ABD PK in Cynomolgus Monkeys

A single 7.5 mg/kg IV dose of PCSK9 adnectin-ABD (SEQ ID NO: 208) was injected in Cynomolgus monkeys and the PK of the protein was measured using an immunoassay that specifically measures adnectin concentrations in plasma. The results indicate that PCSK9 adnectin-ABD has a half-life of 88-94 hours in Cynomolgus monkey, compared to 1-3 hours for the unmodified PCSK9 adnectin (Table 1). Thus, a significant t½ extension was achieved over the non-modified adnectin dosed at 10 mg/kg IV.

### Example 3

### Comparison of PCSK9 Binding Adnectin-HSA Binding Fusion Protein to Other PK Enhanced Adnectins

The half-life in Cynomolgus monkeys and the biological activity of PCSK9 binding adnectin-HSA were compared to those of PCSK9 binding adnectin linked to other PK enhancing moieties. The results are shown in Table 1.

TABLE 1
Comparison of the half-life in Cynomolgus monkeys and the biological
activity of PCSK9 binding adnectin to PCSK9 binding adnectin linked
to different PK enhancers

| | | IV PK Cyno T_{1/2} | EGFA FRET |
|---|---|---|---|
| Enhancer method | | (hr) | (EC₅₀ nM) |
| None | | 1-3 | 2.6 ± 1.2 |
| PEG | 2 br | 84-99 | 2.1 ± 1.1 |
| (40 kDa) | 4 br | 112-123 | 1.9 |

| | | |
|---|---|---|
| Fc-fusion | 67-84 | 0.5 ± 0.3 |
| ABD fusion | 88-94 | 0.30 |
| PKE adnectin | ~130** | — |
| (albumin binder) | | |

**PKE adnectin only

The results indicate that the PK of PCSK9 adnectin linked to ABD in Cynomolgus monkeys and its biological activity compares favorably to PCSK9 adnectin linked to other PK enhancing moieties.

### Example 4

### ABD-(GS)3-C7FL-His6 (Anti-VEGFR2 Adnectin) Binds with High Affinity to VEGFR2 and Albumin and has Good Biophysical Properties

This Example shows that an anti-VEGFR2 adnectin linked to ABD at its N-terminus binds to its target and to serum albumin, and has good biophysical properties.

The following fusion protein was used in this experiment:

(SEQ ID NO: 209)

MGQHDEAVDANSLAEAKVLANRELDKYGVSDYYKNLINNAKTVEGVKALI
DEILAALPGSGSGSVSDVPRDLEVVAATPTSLLISWRHPHFPTRYYRITY
GETGGNSPVQEFTVPLQPPTATISGLKPGVDYTITVYAVTDGRNGRLLSI
PISINYRTGSGSHHHHHH

The binding affinity of the ABD-(GS)3-C7FL-His6 fusion protein was determined by Surface Plasmon Resonance (SPR), wherein VEGFR2 was immobilized on chip and ABD-(GS)3-C7FL-His6 (0.02 nM-300 nM) was flowed as analyte.

The results, which are shown in Table 2, indicate that ABD-(GS)3-C7FL-His6 binds with high affinity to its target, i.e., VEGFR2.

TABLE 2
ABD-C7FL binding to VEGFR2 determined by SPR

| Binding to | ka (1/Ms) | kd (1/s) | KD (nM) | Rmax (RU) |
|---|---|---|---|---|
| sol VEGFR2 | 8.20E+04 | 2.61E−04 | 3.18 | 48.26 |
| VEGFR2-Fc | 4.93E+04 | 1.63E−04 | 3.31 | 58.1 |

The binding affinity of ABD-(GS)3-C7FL-His6 to human, mouse and Cynomolgus monkey serum albumin was also measured. In an SPR experiment, the serum albumins were immobilized on chip, and ABD-(GS)3-C7FL-His6 (0.02 nM-300 nM) was flowed as analyte.

The results, which are shown in Table 3, indicate that ABD-(GS)3-C7FL-His6 binds with high affinity to human, mouse and Cynomolgus monkey serum albumin.

TABLE 3
Cross-species binding to albumins determined by SPR

| | | Binding | ka | kd | KD | Rmax |
|---|---|---|---|---|---|---|
| Adnectin | ATI# | to | (1/Ms) | (1/s) | (nM) | (RU) |
| ABD-C7FL | 1087-103 | HuSA | 4.39E+05 | 5.05E−04 | 1.15 | 179.1 |
| | | RhSA | 4.55E+05 | 8.26E−04 | 1.82 | 130 |
| | | MuSA | 3.31E+05 | 3.30E−04 | 1.00 | 186.5 |

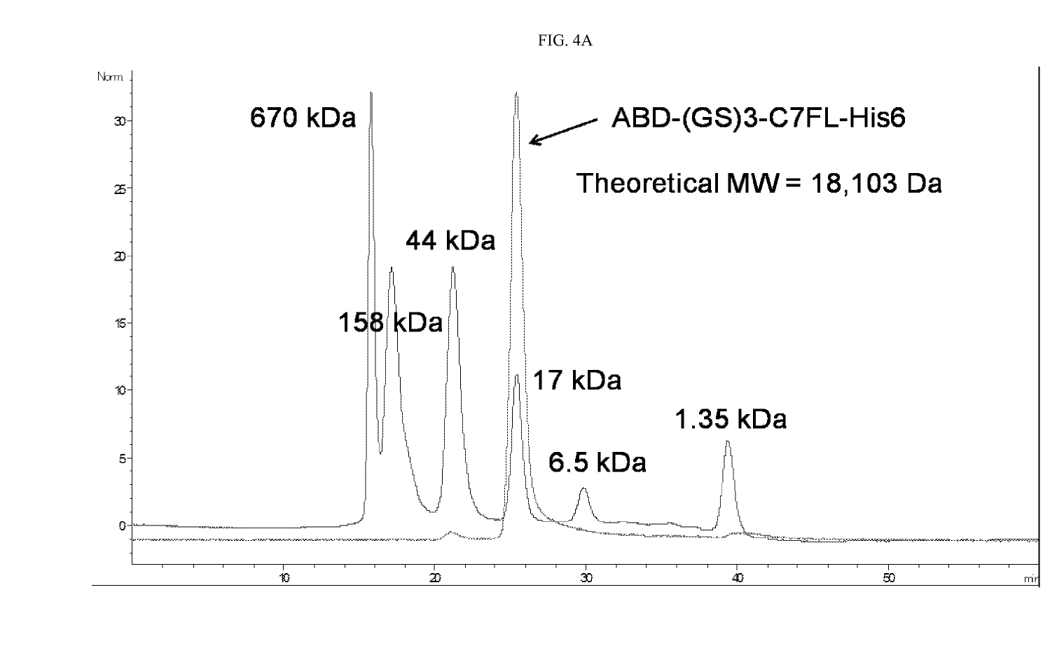

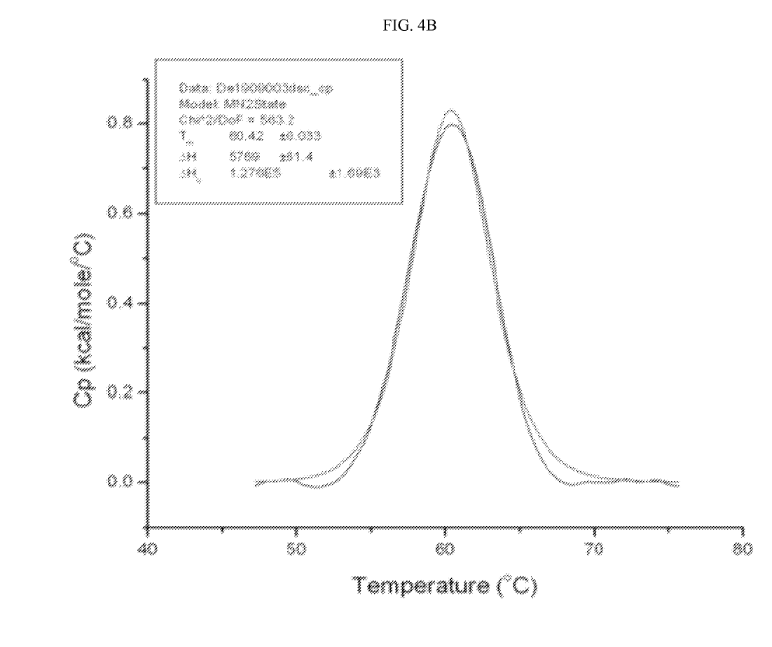

Biophysical characteristics of ABD-(GS)3-C7FL-His6 were also evaluated. In particular, ABD-(GS)3-C7FL-His6 was analyzed by SEC, and was shown to be 99% monomer (FIG. 4A). DSC analysis of ABD-(GS)3-C7FL-His6 indicated that it has a Tₘ of 60.4° C. (FIG. 4B).

Thus, ABD-(GS)3-C7FL-His6 binds to its target, to several species of serum albumin and has good biophysical properties.

### Example 5

### ABD-(GS)3-C7FL has Equivalent Activity as C7FL Alone in the hBaF3 Proliferation Assay with or without HSA

This Example shows that ABD-(GS)3-C7FL-His6 is also active in a biological assay.

The assay was conducted as follows. A cell line that would proliferate in response to human VEGF was constructed by transfection of the murine pre-B cell line Ba/F3 (DSMZ, Braunschweig, Germany). To determine VEGF-induced growth response, cells were seeded on 96-well plates (2-5×104 cells/well) in 95 μL of growth medium. Test protein was added as a 5 μL solution in PBS/20% minimal Ba/F3 medium. After incubation for 72 hours at 37° C., proliferation was measured by the addition of 20 μL of CELLTITER 96® Aqueous One solution (Promega) to each well, followed by measurement of absorbance at 490 nm using a microplate reader (Molecular Dynamics).

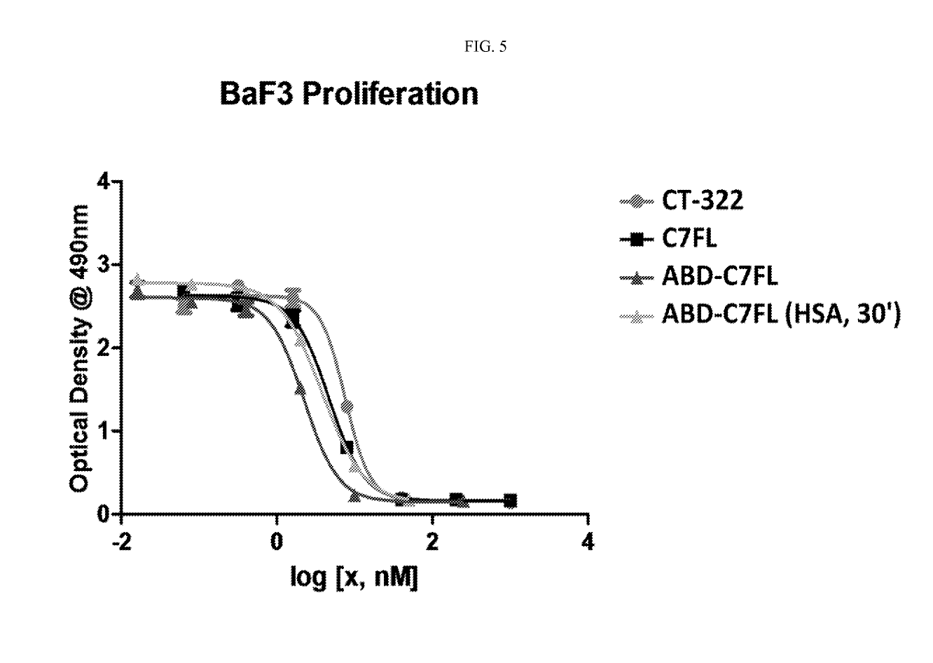

The results, which are shown in FIG. 5 and in Table 4, indicate that ABD-(GS)3-C7FL-His6 has a similar activity to that of the C7FL adnectin alone. Although, it appears to have even more biological activity than the C7FL adnectin alone, a difference less than 3-fold from control (i.e., Rel Pot=0.33<x<3) is not considered significant due to the inherent variability of this assay.

Pre-incubation with 10×HSA does not significantly affect activity indicating that simultaneous binding of the ABD-(GS)3-C7FL-His6 construct with HSA does not prevent engagement with VEGFR2.

Table 4: Table 4: IC₅₀ values for ABD-C7FL and controls +/−HSA in the VEGF hBa/F3 Proliferation Assay. Values are derived from the results in FIG. 5 except for asterisked samples that are not shown. For relative potency (Rel Pot), C7FL minus HSA was set to 1 and the values of the other samples were adjusted accordingly. “CT-322” refers to C7FL lacking the 8 N-terminal amino acids and linked to PEG. “ATI-0001087” refers to ABD-(GS)3-C7FL-His6.

| Material | IC₅₀ | Rel Pot (C7FL) |
|---|---|---|
| CT-322 | 7.533 | 0.62 |
| C7FL | 4.671 | 1.00 |
| ATI-0001087 | 2.228 | 2.10 |
| CT-322 (HSA) | 8.853 | 0.53 |
| C7FL (HSA) | 3.688 | 1.27 |
| ATI-0001087 (HSA) | 3.828 | 1.22 |

Thus, ABD-(GS)3-C7FL-His6 is as biologically active as C7FL that is not linked to ABD.

### Example 6

### ABD-(GS)3-C7FL-His6 has a Good PK (t_{1/2})

The PK of ABD-(GS)3-C7FL-His6 was determined using a MesoScale Discovery platform based method in which ABD-(GS)3-C7FL-His6 was captured from plasma using a monoclonal antibody that specifically recognizes the C7FL adnectin. The fusion protein was detected via an anti-adnectin rabbit polyclonal antibody in combination with a sulfo-tag labeled goat anti-rabbit antibody. Non-compartmental analysis was performed using Pharsight Corporation WINNONLIN® software.

The results indicate that ABD-(GS)3-C7FL-His6 has a PK 32-41 hours in nude mouse and 67-87 hours in Cynomolgus monkey.

### Example 7

### Comparison of PK and Biophysical Properties of ABD-Adnectin Fusions

The PK of the following molecules was determined in nude mice, wildtype mice and Cynomolgus, essentially as described in Example 6 with the exception of the capture reagents which were anti-His mAb for ABD-6×His and RGE-(GS)3-ABD and biotinylated recombinant human PCSK9 for the PCSK9 adnectin containing fusions: ABD-6×His (“ABD-his”), an adnectin, wherein the only change relative to SEQ ID NO: 1 is that the RGD sequence in the FG loop has been replaced with RGE to prevent binding to integrins (“RGE-(GS)3-ABD”); PCSK9 Adn-(GS)₃-ABD; PCSK9 Adn-(ED)₃-ABD; and ABD-(GS)3-C7FL-His6 (“ABD-(GS)₃—C7FL”) that was used in Examples 5 and 6.

The results, which are shown in Table 5, indicate that all molecules have a significant half-life in mouse and Cynomolgus.

TABLE 5
PK of Various Molecules in Mice and Cynomolgus Monkey

| | | WT | |
|---|---|---|---|
| Protein | Nude mouse PK | mouse PK | Cyno PK |
| ABD-his | 40-45 hrs | 52-58 hrs | 190-275 hrs* |
| RGE-(GS)₃-ABD | 40-47 hrs | nd | 132-199 hrs |
| PCSK9 Adn-(GS)₃-ABD | nd | 31-38 hrs | 88-94 hrs |
| PCSK9 Adn-(ED)₃-ABD | nd | 22-28 hrs | nd |
| ABD-(GS)₃-C7FL | 32-41 hrs | nd | 67-87 hrs |

Study not long enough to accurately determine t_{1/2}

The following biophysical characteristics were also measured for the adnectin-ABD fusion proteins: percentage monomer, as determined by SEC; Tm, as measured by DSC; affinity to HSA, as determined by SPR and biological potency relative to that of the adnectin alone.

The results, which are shown in Table 6, indicate that ABD adnectin fusion proteins have significantly prolonged half-lives compared to naked adnectins. Longest t_{1/2} was observed for C7FL, and good enhancement was seen for other adnectins (≧than PEG). ABD-adnectin fusions that bind target have shorter t_{1/2} than ABD alone and cell-receptor targeted construct has shortest half-life, which half-life is analogous to that of PEGylated adnectins.

TABLE 6
Biophysical characteristics of adnectin-ABD fusion proteins

| | | DSC (° C.) (PBS) | | | | | |
|---|---|---|---|---|---|---|---|
| | SEC | | ADNECTIN | | HSA AFFINITY (K_{D}) (nM) | | RELATIVE |
| PROTEIN | (% monomer) | ABD | PARTNER | ABD-FUSION | ABD | ABD-FUSION | POTENCY |
| RGE-(GS)3-ABD | >99 | 75.9 | 83.8 | 74/85 | 1.6-2.6 | 5.09-12.9 | NA |
| (WT-Adnectin) | | | | | | | |
| 2013_E01-(GS)3-ABD | 98 | 75.9 | 81.2 | 73 | 1.6-2.6 | 12.4 | Equivalent |
| (anti-PCSK9) | | | | | | | |
| 2013_E01-(ED)3-ABD | 98 | 75.9 | 81.2 | 77 | 1.6-2.6 | ND | Equivalent |
| (anti-PCSK9) | | | | | | | |
| ABD-(GS)3-C7FL | >99 | 75.9 | 59.9 | 60.4 | 1.6-2.6 | 1.15 | Equivalent |
| (anti-VEGFR2) | | | | | | | |

Thus, adnectin-ABD fusion proteins have an extended PK relative to the adnectin that is not fused to ABD, and good biophysical properties.

The entire disclosure of each document cited (including patents, patent applications, journal articles, abstracts, laboratory manuals, books, GENBANK® Accession numbers, SWISS-PROT® Accession numbers, or other disclosures) in the Background, Detailed Description, Brief Description of the Drawings, and Examples is hereby incorporated herein by reference herein, and where cited in a specific context, includes that particular teaching in the document.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A target binding protein that specifically binds to a target, comprising
(i) an amino acid sequence encoding a fibronectin based scaffold (FBS) moiety that specifically binds to a target; and
(ii) an amino acid sequence encoding a human serum albumin (HSA) moiety,
wherein the target binding protein has one or more improved properties relative to the FBS protein that is not linked to HSA.

2. The target binding protein of claim 1, wherein the one or more improved properties are selected from the group consisting of improved pharmacokinetics, increased shelf-life, increased solubility, increased affinity for the target and increased biological activity.

3. The target binding protein of claim 1 or 2, wherein the FBS moiety comprises a ¹⁰Fn3 moiety.

4. (canceled)

5. (canceled)

6. (canceled)

7. (canceled)

8. The target binding protein of claim 1, wherein the amino acid sequence encoding the FBS moiety and the amino acid sequence encoding the HSA moiety are connected through a linker consisting of at least one amino acid.

9. The target binding protein of claim 8, wherein the linker consists of 1-25 amino acids.

10. The target binding protein of claim 8, wherein the linker is a GS linker.

11. (canceled)

12. (canceled)

13. (canceled)

14. (canceled)

15. (canceled)

16. (canceled)

17. A pharmaceutical composition comprising a target binding protein of claim 1 and a pharmaceutically acceptable carrier.

18. A nucleic acid encoding a target binding protein of claim 1.

19. A target binding protein that specifically binds to a target, comprising
(iii) an amino acid sequence encoding an FBS moiety that specifically binds to a target; and
(iv) an amino acid sequence encoding an albumin binding domain (ABD) moiety,
wherein the target binding protein has one or more improved properties relative to the FBS protein that is not linked to ABD.

20. The target binding protein of claim 19, wherein the one or more improved properties are selected from the group consisting of improved pharmacokinetics, increased shelf-life, increased solubility, increased affinity for the target and increased biological activity.

21. The target binding protein of claim 19, wherein the FBS moiety comprises a ¹⁰Fn3 moiety.

22. The target binding protein of claim 21, wherein the ¹⁰Fn3 domain comprises 1-30 amino acid changes relative to a wild type human ¹⁰Fn3 moiety comprising any one of SEQ ID NOs: 1-16.

23. (canceled)

24. (canceled)

25. (canceled)

26. The target binding protein of claim 19, wherein the amino acid sequence encoding the FBS moiety and the amino acid sequence encoding the ABD moiety are connected through a linker consisting of at least one amino acid.

27. The target binding protein of claim 26, wherein the linker consists of 1-25 amino acids.

28. The target binding protein of claim 26, wherein the linker is a GS linker.

29. (canceled)

30. (canceled)

31. (canceled)

32. (canceled)

33. (canceled)

34. (canceled)

35. The target binding protein of claim 19, which has a half-life in Cynomolgus monkeys that is at least 10 fold as long relative to that of the FBS protein that is not linked to an ABD moiety.

36. The target binding protein of claim 19, which has a half-life in Cynomolgus monkeys of at least 80 hours.

37. The target binding protein of claim 19, wherein the biological activity of the FBS moiety is at least as strong as that of the FBS moiety when it is not linked to an ABD moiety.

38. A pharmaceutical composition comprising a target binding protein of claim 19 and a pharmaceutically acceptable carrier.

39. A nucleic acid encoding a target binding protein of claim 19.
